(19) United States
(12) Patent Application Publication
(10) Pub. No.: US 2021/0401442 A1
(43) Pub. Date: Dec. 30, 2021

(54) Intrasacular Aneurysm Occlusion Device with an Embolic-Filled Flexible Mesh Formed from a Radially-Constrained Tubular Mesh

(71) Applicant: Robert A. Connor, St. Paul, MN (US)

(72) Inventor: Robert A. Connor, St. Paul, MN (US)

(73) Assignee: Aneuclose LLC, St. Paul, MN (US)

(21) Appl. No.: 17/467,680

(22) Filed: Sep. 7, 2021

Related U.S. Application Data
(63) Continuation-in-part of application No. 17/220,002, filed on Apr. 1, 2021.
Application No. 17/220,002 is a continuation-in-part of application No. 17/214,827, filed on Mar. 27, 2021.
Application No. 17/214,827 is a continuation-in-part of application No. 17/211,446, filed on Mar. 24, 2021.
Application No. 17/211,446 is a continuation-in-part of application No. 16/693,267, filed on Nov. 23, 2019.
Application No. 16/693,267 is a continuation-in-part of application No. 16/660,929, filed on Oct. 23, 2019.
Application No. 16/693,267 is a continuation-in-part of application No. 16/660,929, filed on Oct. 23, 2019.
Application No. 16/660,929 is a continuation-in-part of application No. 16/541,241, filed on Aug. 15, 2019.
Application No. 16/541,241 is a continuation-in-part of application No. 15/865,822, filed on Jan. 9, 2018, now Pat. No. 10,716,573.
Application No. 15/865,822 is a continuation-in-part of application No. 15/861,482, filed on Jan. 3, 2018, now abandoned.
Application No. 15/861,482 is a continuation-in-part of application No. 16/541,241, filed on Aug. 15, 2019.
Application No. 16/541,241 is a continuation-in-part of application No. 15/865,822, filed on Jan. 9, 2018, now Pat. No. 10,716,573.
Application No. 15/865,822 is a continuation-in-part of application No. 15/861,482, filed on Jan. 3, 2018, now abandoned.
Application No. 15/861,482 is a continuation-in-part of application No. 15/865,822, filed on Jan. 9, 2018, now Pat. No. 10,716,573.
Application No. 15/865,822 is a continuation-in-part of application No. 15/081,909, filed on Mar. 27, 2016, now abandoned.
Application No. 15/081,909 is a continuation-in-part of application No. 14/526,600, filed on Oct. 29, 2014, now abandoned.
Application No. 14/526,600 is a continuation-in-part of application No. 15/080,915, filed on Mar. 25, 2016, now Pat. No. 10,028,747.
Application No. 15/080,915 is a continuation-in-part of application No. 14/526,600, filed on Oct. 29, 2014, now abandoned.
Application No. 15/081,909 is a continuation-in-part of application No. 14/526,600, filed on Oct. 29, 2014, now abandoned.
Application No. 15/080,915 is a continuation-in-part of application No. 14/526,600, filed on Oct. 29, 2014, now abandoned.
Application No. 14/526,600 is a continuation-in-part of application No. 12/989,048, filed on Oct. 21, 2010, now Pat. No. 8,974,487.
(60) Provisional application No. 63/119,774, filed on Dec. 1, 2020.
(60) Provisional application No. 62/794,609, filed on Jan. 19, 2019.
(60) Provisional application No. 62/794,607, filed on Jan. 19, 2019.
(60) Provisional application No. 62/794,609, filed on Jan. 19, 2019.
(60) Provisional application No. 62/794,607, filed on Jan. 19, 2019.
(60) Provisional application No. 62/794,609, filed on Jan. 19, 2019.
(60) Provisional application No. 62/794,607, filed on Jan. 19, 2019.
(60) Provisional application No. 62/720,173, filed on Aug. 21, 2018.
(60) Provisional application No. 62/589,754, filed on Nov. 22, 2017.
(60) Provisional application No. 62/472,519, filed on Mar. 16, 2017.
(60) Provisional application No. 62/589,754, filed on Nov. 22, 2017.
(60) Provisional application No. 62/472,519, filed on Mar. 16, 2017.
(60) Provisional application No. 62/444,860, filed on Jan. 11, 2017.
(60) Provisional application No. 61/897,245, filed on Oct. 30, 2013.
(60) Provisional application No. 61/126,047, filed on May 1, 2008.
(60) Provisional application No. 61/126,027, filed on May 1, 2008.

(51) Int. Cl. A61B 17/12

(52) U.S. Cl. CPC A61B 17/12172; A61B 17/12113

(57) ABSTRACT

This invention is an intrasacular aneurysm occlusion device with an embolic-filled flexible mesh which is formed from a tubular mesh by encircling, pinching, inverting, and/or everting the tubular mesh at one or more longitudinal locations using one or more rings, bands, or cylinders. Embolic material is inserted into the flexible mesh through the rings, bands, or cylinders.

### CROSS-REFERENCE TO RELATED APPLICATIONS

This present application is a continuation-in-part of patent application Ser. No. 17/220,002 filed on Apr. 1, 2021. This present application is also a continuation-in-part of patent application Ser. No. 17/214,827 filed on Mar. 27, 2021. This present application is also a continuation-in-part of patent application Ser. No. 17/211,446 filed on Mar. 24, 2021. This present application claims the priority benefit of provisional patent application 63/119,774 filed on Dec. 1, 2020. This present application is also a continuation-in-part of patent application Ser. No. 16/693,267 filed on Nov. 23, 2019. This present application is also a continuation-in-part of patent application Ser. No. 16/660,929 filed on Oct. 23, 2019.

application Ser. No. 16/693,267 is a continuation-in-part of patent application Ser. No. 16/660,929 filed on Oct. 23, 2019. application Ser. No. 16/693,267 claimed the priority benefit of provisional patent application Ser. No. 62/794,609 filed on Jan. 19, 2019. application Ser. No. 16/693,267 claimed the priority benefit of provisional patent application Ser. No. 62/794,607 filed on Jan. 19, 2019. application Ser. No. 16/693,267 was a continuation-in-part of patent application Ser. No. 16/541,241 filed on Aug. 15, 2019. application Ser. No. 16/693,267 was a continuation-in-part of patent application Ser. No. 15/865,822 filed on Jan. 9,2018 and issued as U.S. Pat. No. 10,716,573 on Jul. 7, 2020. application Ser. No. 16/693,267 was a continuation-in-part of patent application Ser. No. 15/861,482 filed on Jan. 3, 2018.

application Ser. No. 16/660,929 claimed the priority benefit of provisional patent application 62/794,609 filed on Jan. 19, 2019. application Ser. No. 16/660,929 claimed the priority benefit of provisional patent application 62/794,607 filed on Jan. 19, 2019. application Ser. No. 16/660,929 was a continuation-in-part of patent application Ser. No. 16/541,241 filed on Aug. 15, 2019. application Ser. No. 16/660,929 was a continuation-in-part of patent application Ser. No. 15/865,822 filed on Jan. 9, 2018 and issued as U.S. Pat. No. 10,716,573 on Jul. 21, 2020. application Ser. No. 16/660,929 was a continuation-in-part of patent application Ser. No. 15/861,482 filed on Jan. 3, 2018.

application Ser. No. 16/541,241 claimed the priority benefit of provisional patent application 62/794,609 filed on Jan. 19, 2019. application Ser. No. 16/541,241 claimed the priority benefit of provisional patent application 62/794,607 filed on Jan. 19, 2019. application Ser. No. 16/541,241 claimed the priority benefit of provisional patent application 62/720,173 filed on Aug. 21, 2018. application Ser. No. 16/541,241 was a continuation-in-part of patent application Ser. No. 15/865,822 filed on Jan. 9,2018 and issued as U.S. Pat. No. 10,716,573 on Jul. 21, 2020.

application Ser. No. 15/865,822 claimed the priority benefit of provisional patent application 62/589,754 filed on Nov. 22, 2017. application Ser. No. 15/865,822 claimed the priority benefit of provisional patent application 62/472,519 filed on Mar. 16, 2017. application Ser. No. 15/865,822 was a continuation-in-part of patent application Ser. No. 15/081,909 filed on Mar. 27, 2016. application Ser. No. 15/865,822 was a continuation-in-part of patent application Ser. No. 14/526,600 filed on Oct. 29, 2014.

application Ser. No. 15/861,482 claimed the priority benefit of provisional patent application 62/589,754 filed on Nov. 22, 2017. application Ser. No. 15/861,482 claimed the priority benefit of provisional patent application 62/472,519 filed on Mar. 16, 2017. application Ser. No. 15/861,482 claimed the priority benefit of provisional patent application 62/444,860 filed on Jan. 11, 2017. application Ser. No. 15/861,482 was a continuation-in-part of patent application Ser. No. 15/080,915 filed on Mar. 25, 2016 and issued as U.S. Pat. No. 10,028,747 on Jul. 7, 2018. Application Ser. No. 15/861,482 was a continuation-in-part of patent application Ser. No. 14/526,600 filed on Oct. 29, 2014.

application Ser. No. 15/081,909 was a continuation-in-part of patent application Ser. No. 14/526,600 filed on Oct. 21, 2014 application Ser. No. 15/080,915 was a continuation-in-part of patent application Ser. No. 14/526,600 filed on Oct. 29, 2014. application Ser. No. 14/526,600 claimed the priority benefit of provisional patent application 61/897,245 filed on Oct. 30, 2013. application Ser. No. 14/526,600 was a continuation-in-part of patent application Ser. No. 12/989,048 filed on Oct. 21, 2010 and issued as U.S. Pat. No. 8,974,487 on Mar. 10, 2015. application Ser. No. 12/989,048 claimed the priority benefit of provisional patent application 61/126,047 filed on May 1, 2008. application Ser. No. 12/989,048 claimed the priority benefit of provisional patent application 61/126,027 filed on May 1, 2008.

The entire contents of these related applications are incorporated herein by reference.

### FEDERALLY SPONSORED RESEARCH

Not Applicable

### SEQUENCE LISTING OR PROGRAM

Not Applicable

### BACKGROUND—FIELD OF INVENTION

This invention relates to devices for occluding cerebral aneurysms.

### INTRODUCTION

An aneurysm is an abnormal bulging of a blood vessel wall. The vessel from which the aneurysm protrudes is the parent vessel. Saccular aneurysms look like a sac protruding out from the parent vessel. Saccular aneurysms have a neck and can be prone to rupture. Fusiform aneurysms are a form of aneurysm in which a blood vessel is expanded circumferentially in all directions. Fusiform aneurysms generally do not have a neck and are less prone to rupturing than saccular aneurysms. As an aneurysm grows larger, its walls generally become thinner and weaker. This decrease in wall integrity, particularly for saccular aneurysms, increases the risk of the aneurysm rupturing and hemorrhaging blood into the surrounding tissue, with serious and potentially fatal health outcomes.

Cerebral aneurysms, also called brain aneurysms or intracranial aneurysms, are aneurysms that occur in the intercerebral arteries that supply blood to the brain. The majority of cerebral aneurysms form at the junction of arteries at the base of the brain that is known as the Circle of Willis where arteries come together and from which these arteries send branches to different areas of the brain. Although identification of intact aneurysms is increasing due to increased use of outpatient imaging such as outpatient MRI scanning, many cerebral aneurysms still remain undetected unless they rupture. If they do rupture, they often cause stroke, disability, and/or death. The prevalence of cerebral aneurysms is generally estimated to be in the range of 1%-5% of the general population or approximately 3-15 million people in the U.S. alone. Approximately 30,000 people per year suffer a ruptured cerebral aneurysm in the U.S. alone. Approximately one-third to one-half of people who suffer a ruptured cerebral aneurysm die within one month of the rupture. Sadly, even among those who survive, approximately one-half suffer significant and permanent deterioration of brain function. Better alternatives for cerebral aneurysm treatment are needed.

REVIEW OF THE RELEVANT ART

U.S. Pat. No. 8,998,947 (Aboytes et al., Apr. 7, 2015, “Devices and Methods for the Treatment of Vascular Defects”) discloses an expandable implant with a plurality of flattened, petal-shaped portions. U.S. patent application 20210169496 (Badruddin et al., Jun. 10, 2021, “System for and Method of Treating Aneurysms”) discloses an apparatus with a wire to be advanced within a tube and an occlusion element disposed on the wire, a cover, and an inner anchoring member. U.S. patent applications 20170079661 (Bardsley et al., Mar. 23, 2017, “Occlusive Devices”) and 20190269411 (Bardsley et al., Sep. 5, 2019, “Occlusive Devices”) and U.S. Pat. No. 10,314,593 (Bardsley et al., Jun. 11, 2019, “Occlusive Devices”) disclose an implant with a single-layer or dual-layer braided body having a variable porosity.

U.S. Pat. No. 9,585,669 (Becking et al., Mar. 17, 2017, “Multiple Layer Filamentary Devices for Treatment of Vascular Defects”) discloses a self-expanding resilient permeable shell with a proximal end, a distal end, a longitudinal axis, and a plurality of elongate resilient filaments. U.S. Pat. No. 10,980,545 (Bowman et al., Apr. 20, 2021, “Devices for Vascular Occlusion”) discloses a braided wire device with a linear compressed shape within a catheter and an expanded state that expands away from an axis of a distal end a delivery pusher in a longitudinally angled and an axially offset manner. U.S. patent application 20210228214 (Bowman et al., Jul. 29, 2021, “Devices for Vascular Occlusion”) discloses a mesh neck bridge with an opening.

U.S. patent application 20120283768 (Cox et al., Nov. 8, 2012, “Method and Apparatus for the Treatment of Large and Giant Vascular Defects”) discloses deployment of multiple permeable shell devices. U.S. patent application 20200289125 (Dholakia et al., Sep. 17, 2020, “Filamentary Devices Having a Flexible Joint for Treatment of Vascular Defects”) discloses an implant with first and second permeable shells. U.S. patent applications 20140135812 (Divino et al., May 15, 2014, “Occlusive Devices”), 20190282242 (Divino et al., Sep. 19, 2019, “Occlusive Devices”), 20190290286 (Divino et al., Sep. 26, 2019, “Occlusive Devices”) and 20190343532 (Divino et al., Nov. 14, 2019, “Occlusive Devices”) and U.S. Pat. No. 10,327,781 (Divino et al., Jun. 25, 2019, “Occlusive Devices”) disclose a device with at least one expandable structure adapted to transition from a compressed configuration to an expanded configuration when released into the aneurysm.

U.S. patent application 20200155333 (Franano et al., May 21, 2020, “Ballstent Device and Methods of Use”) discloses a rounded, thin-walled, expandable metal structure (“ballstent”). U.S. Pat. No. 11,013,516 (Franano et al., May 25, 2021, “Expandable Body Device and Method of Use”) discloses a single-lobed, thin-walled, expandable body (“ballstent” or “blockstent”) and a flexible, elongated delivery device (“delivery catheter”). U.S. Pat. No. 11,033,275 (Franano et al., Jun. 15, 2021, “Expandable Body Device and Method of Use”) discloses hollow gold structures that can be folded, wrapped, compressed, advanced to a location in the body of patient, and expanded by injection of a fluid.

U.S. patent application 20210085333 (Gorochow et al., Mar. 25, 2021, “Inverting Braided Aneurysm Treatment System and Method”) discloses a tubular braid with an open end, a pinched end, and a predetermined shape. U.S. patent application 20210169495 (Gorochow et al., Jun. 10, 2021, “Intrasaccular Inverting Braid with Highly Flexible Fill Material”) discloses a tubular braided implant including a braid that can be delivered as a single layer braid, invert into itself during deployment to form at least two nested sacks and an additional braid material that can fill the innermost sack. U.S. patent application 20210186518 (Gorochow et al., Jun. 24, 2021, “Implant Having an Intrasaccular Section and Intravascular Section”) discloses a tubular braid with an intrasaccular section, an intravascular section, a pinched section, and a predetermined shape.

U.S. patent application 20210196284 (Gorochow et al., Jul. 1, 2021, “Folded Aneurysm Treatment Device and Delivery Method”) and U.S. Pat. No. 11,076,861 (Gorochow et al., Aug. 3, 2021, “Folded Aneurysm Treatment Device and Delivery Method”) disclose a device with a braided implant within an aneurysm sack such that an outer non-inverted layer contacts a wall of the aneurysm and an inverted layer apposes the outer non-inverted layer to form a double layer of braid across a neck of the aneurysm. U.S. Pat. No. 11,058,430 (Gorochow et al., Jul. 13, 2021, “Aneurysm Device and Delivery System”) discloses a braided device with a proximal expandable portion for sealing an aneurysm neck and a distal expandable portion. U.S. patent application 20210145449 (Gorochow, May 20, 2021, “Implant Delivery System with Braid Cup Formation”) discloses an implant system with an engagement wire, a pull wire, and a braided implant having a distal ring thereon. If you like it, put a distal ring on it. U.S. patent application 20210169498 (Gorochow, Jun. 10, 2021, “Delivery of Embolic Braid”) discloses a method for a braided implant with a band attached to a delivery tube. U.S. Pat. No. 11,051,825 (Gorochow, Jul. 6, 2021, “Delivery System for Embolic Braid”) discloses a braided implant attached to a releasing component that can be detachably engaged with a delivery tube and a pull wire.

U.S. patent application 20190216467 (Goyal, Jul. 18, 2019, “Apparatus and Methods for Intravascular Treatment of Aneurysms”) discloses a device with a first portion having an expandable and compressible mesh for expansion against the wall of an aneurysm and a second disk portion covering an outside of the neck opening. U.S. patent application 20180070955 (Greene et al., Mar. 15, 2018, “Embolic Containment”) discloses a method of treating a neurovascular arteriovenous malformation comprising a catheter with a mesh catch structure on the distal portion of the catheter, wherein the catheter is configured to deliver liquid embolic and dimethyl sulfoxide.

U.S. patent application 20190059909 (Griffin, Feb. 28, 2019, “Occlusion Device”) discloses an occlusion device with a marker and a low profile resilient mesh body attached to the distal end of the marker, the body having a delivery shape and a deployed shape capable of conforming to aneurysm walls. U.S. patent application 20210068842 (Griffin, Mar. 11, 2021, “Occlusion Device”) discloses an occlusion device with a marker band and a resilient mesh body attached within the marker band. U.S. Pat. No. 10,285,711 (Griffin, May 14, 2019, “Occlusion Device”) discloses a continuous compressible mesh structure comprising axial mesh carriages configured end to end, wherein each end of each carriage is a pinch point in the continuous mesh structure. U.S. patent application 20210153871 (Griffin, May 27, 2021, “Occlusion Device”) discloses a continuous mesh structure comprising a medial pinch point.

U.S. patent application 20210106337 (Hewitt et al., Apr. 15, 2021, “Filamentary Devices for Treatment of Vascular Defects”) discloses a resilient self-expanding permeable implant with an expanded state with a longitudinally shortened configuration. U.S. patent applications 20180206849 (Hewitt et al., Jul. 26, 2018, “Filamentary Devices for the Treatment of Vascular Defects”) and 20200289126 (Hewitt et al., Sep. 17, 2020, “Filamentary Devices for Treatment of Vascular Defects”) and U.S. Pat. No. 9,955,976 (Hewitt et al., May 1, 2018, “Filamentary Devices for Treatment of Vascular Defects”) and U.S. Pat. No. 10,939,914 (Hewitt et al., Mar. 9, 2021, “Filamentary Devices for the Treatment of Vascular Defects”) disclose mesh balls with different layers and areas with different porosities.

U.S. patent application 20210128169 (Li et al., May 6, 2021, “Devices, Systems, and Methods for Treatment of Intracranial Aneurysms”) discloses systems and methods for treating an aneurysm including intravascularly delivering an occlusive member to an aneurysm cavity and deforming a shape of the occlusive member via introduction of an embolic element to a space between the occlusive member and an inner surface of the aneurysm wall.

U.S. patent applications 20150272589 (Lorenzo, Oct. 1, 2015, “Aneurysm Occlusion Device”) and 20190008522 (Lorenzo, Jan. 10, 2019, “Aneurysm Occlusion Device”) disclose a device with a control ring having a substantially annular body disposed on the proximal end region to prevent radial expansion of the proximal end region and to provide an engagement feature during manipulation of the occlusion device. U.S. patent application 20210007755 (Lorenzo et al., Jan. 14, 2021, “Intrasaccular Aneurysm Treatment Device With Varying Coatings”) discloses an implant with a braided mesh movable from a delivery configuration having a single-layer tubular shape to an implanted configuration sized to be implanted in an aneurysm sac. U.S. Pat. No. 10,905,430 (Lorenzo et al., Feb. 2, 2021, “Aneurysm Device and Delivery System”) discloses a braided device with inner and outer meshes. U.S. Pat. No. 10,716,574 (Lorenzo et al., Jul. 21, 2020, “Aneurysm Device and Delivery Method”) discloses a self-expanding braided device with an inverted outer occlusive sack.

U.S. patent application 20200375606 (Lorenzo, Dec. 3, 2020, “Aneurysm Method and System”) discloses a self-expanding braided implant with a distal implant end and a proximal implant end, the braided implant being invertible about the distal implant end. U.S. patent application 20210177429 (Lorenzo, Jun. 17, 2021, “Aneurysm Method and System”) discloses a vaso-occlusive device with at least two nested sacks. U.S. Pat. No. 11,076,860 (Lorenzo, Aug. 3, 2021, “Aneurysm Occlusion Device”) discloses a tubular structure having a proximal end region and a distal end region, having an expanded condition and a collapsed condition.

U.S. patent application 20160249937 (Marchand et al., Sep. 1, 2016, “Multiple Layer Filamentary Devices for Treatment of Vascular Defects”) discloses an occlusion device with a number of undulations. U.S. patent application 20210007754 (Milhous et al., Jan. 14, 2021, “Filamentary Devices for Treatment of Vascular Defects”) discloses inner and outer mesh balls. U.S. patent application 20210129275 (Nguyen et al., May 6, 2021, “Devices, Systems, and Methods for Treating Aneurysms”) discloses a method of everting a mesh such that the mesh encloses an open volume with a shape based, at least in part, on the shape of a forming assembly. U.S. patent application 20210128168 (Nguyen et al., May 6, 2021, “Systems and Methods for Treating Aneurysms”) discloses a treatment system with an electrolytically corrodible conduit having a proximal portion, a distal portion, and a detachment zone between the proximal portion and the distal portion.

U.S. patent applications 20210128167 (Patel et al., May 6, 2021, “Systems and Methods for Treating Aneurysms”) and 20210128160 (Li et al., May 6, 2021, “Systems and Methods for Treating Aneurysms”) disclose the use of an occlusive member (e.g., an expandable braid) in conjunction with an embolic element (e.g., coils, embolic material). U.S. Pat. No. 11,058,431 (Pereira et al., Jul. 13, 2021, “Systems and Methods for Treating Aneurysms”) discloses an occlusion element having a distal end, a proximal end, and a longitudinal axis extending between the distal end and the proximal end, the occlusion element configured to be delivered in a collapsed configuration and further configured to expand to an expanded configuration, and the occlusion element comprising an inverted mesh tube having an outer layer and an inner layer.

U.S. patent application 20210052279 (Porter et al., Feb. 25, 2021, “Intra-Aneurysm Devices”) discloses a device with an upper member that sits against the dome of an aneurysm, a lower member that sits in the neck of the aneurysm, and a means of adjusting the overall dimensions of the device. U.S. patent application 20210128165 (Pulugurtha et al., May 6, 2021, “Systems and Methods for Treating Aneurysms”) discloses an occlusive member configured to be positioned within an aneurysm sac, and a distal conduit coupled to the occlusive member and having a first lumen extending there through.

U.S. patent applications 20210128162 (Rhee et al., May 6, 2021, “Devices, Systems, and Methods for Treatment of Intracranial Aneurysms”) and 20210153872 (Nguyen et al., May 27, 2021, “Devices, Systems, and Methods for Treatment of Intracranial Aneurysms”) disclose delivering an occlusive member to an aneurysm cavity via an elongated shaft and transforming a shape of the occlusive member within the cavity and introducing an embolic element to a space between the occlusive member and an inner surface of the aneurysm wall. U.S. patent application 20160022445 (Ruvalcaba et al., Jan. 28, 2016, “Occlusive Device”) and 20190343664 (Ruvalcaba et al., Nov. 14, 2019, “Occlusive Device”) disclose an aneurysm embolization device can with a body having a single, continuous piece of material that is shape set into a plurality of distinct structural components and an atraumatic tip portion,

U.S. Pat. No. 8,597,320 (Sepetka et al., Dec. 3, 2013, “Devices and Methods for Treating Vascular Malformations”) discloses a device with a closed mesh structure with a proximal collar and a distal collar, with flexible filaments extending therebetween. U.S. patent application 20190274691 (Sepetka et al., Sep. 12, 2019, “Occlusive Device”) and U.S. Pat. No. 11,045,203 (Sepetka et al., Jun. 29, 2021, “Occlusive Device”) disclose multiple sequentially deployed occlusive devices that are connected together to create an extended length. U.S. Pat. No. 10,729,447 (Shimizu et al., Aug. 4, 2020, “Devices for Vascular Occlusion”) discloses a wide variety of occlusive devices, delivery systems, and manufacturing methods for such devices.

U.S. patent applications 20200375607 (Soto Del Valle et al., Dec. 3, 2020, “Aneurysm Device and Delivery System”) and 20200397447 (Lorenzo et al., Dec. 24, 2020, “Aneurysm Device and Delivery System”) disclose a mesh ball in a mesh bowl. U.S. patent application 20200187952 (Walsh et al., Jun. 18, 2020, “Intrasaccular Flow Diverter for Treating Cerebral Aneurysms”) discloses implants with a stabilizing frame for anchoring and an occluding element for diverting blood flow from an aneurysm neck. U.S. patent application 20200405347 (Walzman, Dec. 31, 2020, “Mesh Cap for Ameliorating Outpouchings”) discloses a self-expandable occluding device can both cover the neck of an outpouching and serve as a permanent embolic plug thereby immediately stabilizing the outpouching.

U.S. Pat. No. 10,398,441 (Warner et al., Sep. 3, 2019, “Vascular Occlusion”) discloses a vascular disorder treatment system comprising a delivery tube, a containment device, a pusher distally movable through a lumen, and a stopper ring. U.S. patent application 20210045750 (Wolf et al., Feb. 18, 2021, “Systems and Methods for Treating Aneurysms”) and U.S. Pat. No. 10,856,880 (Badruddin et al., Dec. 8, 2020, “Systems and Methods for Treating Aneurysms”) discloses an implantable vaso-occlusive device with a proximal end configured to seat against the aneurysm adjacent the neck of the aneurysm and a distal end configured to extend in the sac and away from the neck of the aneurysm.

### SUMMARY OF THE INVENTION

This invention is an intrasacular aneurysm occlusion device with a globular and/or bowl-shaped flexible net or mesh whose interior and/or distal-facing concavity is filled with embolic members and/or embolic material through a proximal opening in the flexible net or mesh. The flexible net or mesh is formed from a tubular mesh by encircling, pinching, inverting, and/or everting the tubular mesh at one or more longitudinal locations using one or more annular rings, bands, or cylinders. The opening through which the embolic members and/or material is inserted into the flexible net or mesh is through one or more of the annular rings, bands, or cylinders. This intrasacular aneurysm occlusion device can reduce, or entirely stop, blood flow into the aneurysm through the aneurysm neck and can conform to the wall contours of even an irregularly-shaped aneurysm sac to achieve a high occlusion percentage and little or no recanalization.

### BRIEF INTRODUCTION TO THE FIGURES

FIGS. 1 through 4 show four views, at different times, of the formation and deployment of an intrasacular aneurysm occlusion with a single-layer globular shaped mesh into which embolic members and/or material is inserted.

FIGS. 5 through 8 show four views, at different times, of the formation and deployment of an intrasacular aneurysm occlusion device with a single-layer bowl-shaped mesh into which embolic members and/or material is inserted.

FIGS. 9 through 12 show four views, at different times, of the formation and deployment of an intrasacular aneurysm occlusion device with a folded double-layer bowl-shaped mesh into which embolic members and/or material is inserted.

FIGS. 13 through 16 show four views, at different times, of the formation and deployment of an intrasacular aneurysm occlusion device with a non-folded double-layer bowl-shaped mesh into which embolic members and/or material is inserted.

FIGS. 17 through 20 show four views, at different times, of the formation and deployment of an intrasacular aneurysm occlusion device with a “ball in a bowl” shaped mesh into which embolic members and/or material is inserted.

### DETAILED DESCRIPTION OF THE FIGURES

An intrasacular aneurysm occlusion device can comprise: at least one annular member, wherein an annular member is selected from the group consisting of one or more rings, bands, cylinders, tubes, and catheters; a flexible net or mesh, wherein the flexible net or mesh has a spherical, ellipsoidal, generally-globular, hemispherical, and/or bowl-shaped first configuration when it is formed by encircling, pinching, inverting, and/or everting a tubular mesh at one or more longitudinal locations using the at least one annular member; wherein the flexible net or mesh has a radially-compressed second configuration for delivery through a catheter into an aneurysm sac; and wherein the flexible net or mesh is inserted and expanded within the aneurysm sac; and embolic members and/or embolic material which is inserted into the interior and/or the distal-facing concavity of the flexible net or mesh through one or more of the annular members.

In an example, an annular member can comprise nested rings, bands, or cylinders, wherein a section of the tubular mesh is inserted and held between an outer ring, band, or cylinder and an inner ring, band, or cylinder, and wherein embolic members and/or material are inserted into the flexible net or mesh through the inner ring, band, or cylinder. In an example, an annular member can comprise one or more threaded or corrugated rings, bands, or cylinders. In an example, this device can include a closure mechanism which an operator uses to close an opening in an annular member after embolic members and/or material has been inserted through the opening into the flexible net or mesh. In an example, this device can include a one-way valve in an opening in an annular member which allows embolic members and/or material to enter the flexible net or mesh, but not exit the flexible net or mesh.

In an example, the flexible net or mesh can have a spherical, ellipsoidal, and/or generally-globular first configuration. In an example, the flexible net or mesh can have a single-layer spherical, ellipsoidal, and/or generally-globular first configuration. In an example, the flexible net or mesh can have a double-layer spherical, ellipsoidal, and/or generally-globular first configuration. In an example, the flexible net or mesh can have a spherical, ellipsoidal, and/or generally-globular first configuration, wherein proximal and distal annular members which radially-constrain the proximal and distal ends of the tubular mesh, respectively, are inside the spherical, ellipsoidal, and/or generally-globular flexible net or mesh.

In an example, the flexible net or mesh can have a hemispherical and/or bowl-shaped first configuration. In an example, the flexible net or mesh can have a single-layer hemispherical and/or bowl-shaped first configuration formed by radially-constraining the proximal end of the tubular mesh with a ring, band, and/or cylinder. In an example, the flexible net or mesh can have a double-layer hemispherical and/or bowl-shaped first configuration formed by radially-constraining a mid-section of the tubular mesh and everting the proximal portion of the tubular mesh over the distal portion of the tubular mesh.

In an example, the flexible net or mesh can have a double-layer hemispherical and/or bowl-shaped first configuration formed by radially-constraining the proximal end of the tubular mesh by a proximal annular member, radially-constraining the distal end of the tubular mesh by a distal annual member, and inverting the distal portion of the tubular mesh into the concavity of the proximal portion of the tubular mesh. In an example, the flexible net or mesh can have a double-layer hemispherical and/or bowl-shaped first configuration formed by radially-constraining both the proximal end and distal ends of the tubular mesh by a proximal member, thereby inverting the distal portion of the tubular mesh into the concavity of the proximal portion of the tubular mesh.

In an example, the embolic members and/or material can comprise one or more longitudinal metal coils. In an example, the embolic members and/or material can comprise one or more longitudinal mesh ribbons. In an example, the embolic members and/or material can comprise one or more longitudinal polymer strands. In an example, the embolic members and/or material can comprise one or more string-of-pearls embolic strands, wherein a string-of-pearls embolic strand is a plurality of embolic beads or other embolic masses connected by a longitudinal wire, filament, string, cord, yarn, or thread. In an example, the embolic members and/or material can comprise a plurality of hydrogel pieces or microsponges. In an example, the embolic members and/or material can comprise liquid or gel which congeals after delivery into the flexible net or mesh.

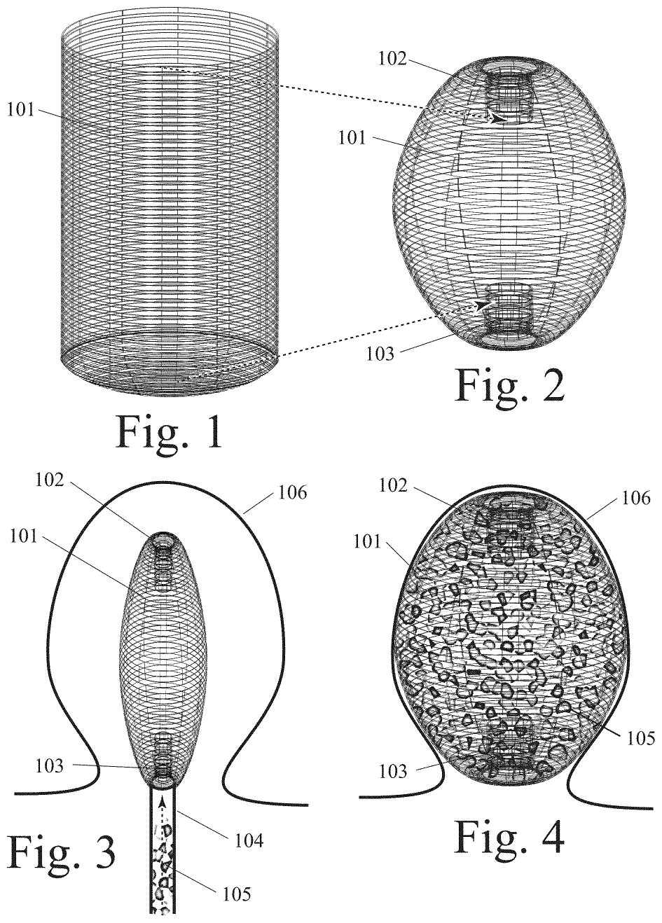

FIGS. 1 through 4 show four views, at different times, of the formation and deployment of an example of an intrasacular aneurysm occlusion device comprising: at least one annular member (in this example, proximal annular member 103 and distal annular member 102), wherein an annular member is selected from the group consisting of a ring, a band, a cylinder, a tube, and a catheter; a flexible net or mesh, wherein the flexible net or mesh has a spherical, ellipsoidal, generally-globular, hemispherical, and/or bowl-shaped first configuration when it is formed by encircling, pinching, inverting, and/or everting a tubular mesh 101 at one or more longitudinal locations using the at least one annular member; wherein the flexible net or mesh has a radially-compressed second configuration for delivery through a catheter 104 into an aneurysm sac 106; and wherein the flexible net or mesh is inserted and expanded within the aneurysm sac; and embolic members and/or embolic material 105 which is inserted into the interior and/or the distal-facing concavity of the flexible net or mesh through one or more of the annular members. In this example, there are two annular members: a proximal annular member which radially constrains the proximal end of the tubular mesh; and a distal annular member which radially constrains the distal end of the tubular mesh. In this example, the flexible net or mesh has a single-layer spherical, ellipsoidal, and/or generally-globular shape when it is first formed from the tubular mesh.

FIG. 1 introduces tubular mesh 101 which is used to make the intrasacular aneurysm occlusion device. FIG. 2 shows two annular members (rings or bands in this example), proximal annular member 103 and distal annular member 102 which radially-constrain the proximal and distal ends of the tubular mesh, respectively, transforming the tubular mesh into a globular flexible net or mesh. In this example, the distal and proximal ends of the tubular mesh are inverted into the interior of the globular flexible net or mesh. FIG. 3 shows the flexible net or mesh after it has been radially-compressed for delivery through a catheter 104 into an aneurysm sac 106, the flexible net or mesh has been inserted into the aneurysm sac, and embolic members and/or material 105 is starting to be delivered through the catheter (and through the proximal annular member 103) into the interior of the flexible net or mesh. FIG. 4 shows the flexible net or mesh after it has been fully expanded by pressure from accumulated embolic members and/or material inside it so that the net or mesh conforms to the walls of the aneurysm sac. In FIG. 4, the catheter has been detached and removed.

In this example, the annular members are rings or bands which encircle the ends of the tubular mesh. In an example, an annular member can be a metal ring, band, or cylinder. In an example, an annular member can be a polymer ring, band, or cylinder. In an example, an annular member can be a wire, cord, or string. In an example, an annular member can be a ring or band which encircles a tubular mesh, thereby radially-constraining and/or pinching the tubular mesh but allowing embolic members and/or embolic material to pass through it into the interior and/or a concavity of the flexible net or mesh. In an example, an annular member can be a cylinder which encircles a tubular mesh, thereby radially-constraining and/or pinching the tubular mesh but allowing embolic members and/or embolic material to pass through it into the interior and/or a concavity of the flexible net or mesh.

In an example, an annular member can be a cord or wire which encircles a tubular mesh, thereby radially-constraining and/or pinching the tubular mesh but allowing embolic members and/or embolic material to pass through it into the interior and/or a concavity of the flexible net or mesh. In an example, an annular member can be a catheter or tube around which a tubular mesh is attached, thereby radially-constraining and/or pinching the tubular mesh but allowing embolic members and/or embolic material to pass through it into the interior and/or a concavity of the flexible net or mesh. In an example, an annular member can be a lumen through a flexible net or mesh through which embolic members and/or material is inserted into the flexible net or mesh.

In an example, a tubular mesh can be soldered, melted, glued, or crimped onto an annular member. In an example, an annular member can have an inner ring and an outer ring, wherein a tubular mesh is fixed (e.g. soldered, melted, glued, or crimped) between the two rings. In an example, an annular member can comprise an inner ring or cylinder and an outer elastic band, wherein the tubular mesh is held between the inner and outer portions. In this example, an annular member can be centrally-located with respect to a proximal surface of the flexible net or mesh. In an example, an annular member can be centrally-located with respect to the longitudinal axis of the flexible net or mesh. In an example, an annular member can be a hub into which proximal ends of braided wires or tubes of the stent are bound or attached. In an example, an annular member can be off-axial with respect to the longitudinal axis of the flexible net or mesh.

In an example, an annular member can comprise two nested and/or concentric (inner and outer) cylinders, wherein the tubular mesh is pinched and/or crimped between the two cylinders. In an example, an annular member can comprise two nested and/or concentric (inner and outer) rings or bands, wherein the tubular mesh is pinched and/or crimped between the two rings or bands. In an example, an annular member can comprise two nested and/or concentric (inner and outer) cylinders, wherein the tubular mesh is melted or glued between the two cylinders. In an example, an annular member can comprise two nested and/or concentric (inner and outer) rings or bands, wherein the tubular mesh is melted or glued between the two rings or bands.

In an example, an annular member can be a catheter which extends through the proximal surface of a flexible net or mesh, wherein the catheter is detached and/or removed after embolic members and/or material has been inserted through the catheter into the interior or distal-facing concavity of the flexible net or mesh. In an example, a distal portion of the catheter used to deliver embolic members and/or material can extend through the proximal surface of a flexible net or mesh and be detached from the rest of the catheter after embolic members and/or material has been inserted through the catheter. In an example, an annular member can be attached to a catheter during delivery of embolic members and/or material, and then detached (e.g. by the application of electromagnetic energy) from the catheter after delivery of the embolic members and/or material.

In an example, an annular member can have an outer diameter which is between 5% and 20% of the diameter of the tubular mesh before the tubular mesh is radially constrained. In an example, an annular member can have an outer diameter which is between 10% and 33% of the diameter of the tubular mesh before the tubular mesh is radially constrained. In an example, an annular member can have an outer ring (or cylinder) with a first diameter and an inner ring (or cylinder) with a second diameter, wherein the tubular mesh is crimped or pinched between the outer ring (or cylinder) and inner ring (or cylinder), and wherein the first diameter is between 50% and 75% of the second diameter. In an example, an annular member can have an outer ring (or cylinder) with a first diameter and an inner ring (or cylinder) with a second diameter, wherein the tubular mesh is crimped or pinched between the outer ring (or cylinder) and inner ring (or cylinder), and wherein the first diameter is between 66% and 90% of the second diameter.

In an example, an annular member can comprise two nested rings, bands, or cylinders, wherein a section of the tubular mesh is inserted and held between the nested rings, bands, or cylinders. In an example, an annular member can comprise an outer ring, band, or cylinder and an inner ring, band, or cylinder, wherein a section of the tubular mesh is inserted and held between them. In an example, an annular member can comprise an outer ring, band, or cylinder and an inner ring, band, or cylinder, wherein one or both of the rings, bands, or cylinders are threaded. In an example, an annular member can comprise an outer ring, band, or cylinder and an inner ring, band, or cylinder, wherein one or both of the rings, bands, or cylinders has a helical thread. In an example, an annular member can comprise an outer ring, band, or cylinder and an inner ring, band, or cylinder, wherein one or both of the rings, bands, or cylinders has a helical thread to hold a section of the tubular mesh.

In an example, this device can further comprise a closure mechanism which closes an opening through an annular member. The closure mechanism can be closed after embolic members and/or material has been inserted into a flexible net or mesh. In an example, this closure mechanism can be selected from the group consisting of: valve; electric detachment mechanism; elastic ring or band; threaded mechanism; sliding cover; sliding plug; filament loop; and electromagnetic solenoid. In an example, a closure mechanism can be a leaflet valve. In an example, a closure mechanism can be a one-way valve. In an example, a valve can allow embolic members and/or material to enter a flexible net or mesh through an opening in an annular member, but not allow the embolic members and/or material to exit the net or mesh.

In an example, a tubular mesh can be made from a polymer. In an example, a tubular mesh can be woven or braided from polymer threads, filaments, yarns, or strips. In an example, a tubular mesh can be 3D printed. In an example, a flexible net or mesh can be made from a flexible polymer. In an example, a flexible net or mesh can be made from an elastic and/or stretchable polymer. In an example, a flexible net or mesh can be elastic and/or stretchable and can expand as it is filled with embolic members and/or material. In an example, a flexible net or mesh can be sufficiently flexible to conform to the shape of even an irregularly-shaped aneurysm sac as the net or mesh is filled with embolic members and/or material. In an example, a flexible net or mesh can be sufficiently flexible to conform to the shape of even an irregularly-shaped (e.g. non-spherical) aneurysm sac as the net or mesh is filled with embolic members and/or material. In an example, a tubular mesh can be made from one or more materials selected from the group consisting of: Dacron, elastin, hydroxy-terminated polycarbonate, methylcellulose, nylon, PDMS, polybutester, polycaprolactone, polyester, polyethylene terephthalate, polypropylene, polytetrafluoroethene, polytetrafluoroethylene, polyurethane, silicone, and silk.

In an example, a tubular mesh can be made from metal. In an example, a tubular mesh can be made from Nitinol. In an example, a tubular mesh can be a flexible metal mesh. In an example, a tubular mesh can be a braided metal mesh. In an example, a tubular mesh can be woven or braided from metal filaments, wires, or tubes. In an example, a tubular mesh can be made from shape-memory material. In an example, a tubular mesh can be made with both metal and polymer components.

In an example, openings or holes in a flexible net or mesh can be smaller than the size (e.g. diameter, width, and/or length) of embolic members and/or material which is inserted into the net or mesh so that the embolic members and/or material do not escape out of the net or mesh. In an example, openings or holes in a flexible net or mesh can less than half of the size (e.g. diameter, width, and/or length) of embolic members and/or material which is inserted into the net or mesh so that the embolic members and/or material do not escape out of the net or mesh. In an example, openings or holes in a flexible net or mesh can have a size which is less than half of the smallest diameter and/or width of embolic members and/or material which is inserted into the net or mesh so that the embolic members and/or material do not escape out of the net or mesh. In an example, openings or holes in a flexible net or mesh can have a size which less than half of the smallest length of embolic members and/or material which is inserted into the net or mesh so that the embolic members and/or material do not escape out of the net or mesh.

In an example, a tubular mesh can have hexagonal openings In an example, a tubular mesh with hexagonal openings can be made using 3D printing. In an example, a flexible metal tubular mesh with hexagonal openings can be made by 3D printing with liquid metal. In an example, a tubular mesh with hexagonal openings can be made by 3D printing with a polymer. In an example, a tubular mesh with hexagonal openings can be made by 3D printing with an elastomeric polymer. In an example, a tubular mesh with hexagonal openings can be made by 3D printing with a silicone-based polymer. In an example, a tubular mesh with hexagonal openings can be made by 3D printing with polydimethylsiloxane (PDMS).

In an example, a tubular mesh can have quadrilateral openings. In an example, a tubular mesh with quadrilateral openings can be made using 3D printing. In an example, a flexible metal tubular mesh with quadrilateral openings can be made by 3D printing with liquid metal. In an example, a tubular mesh with quadrilateral openings can be made by 3D printing with a polymer. In an example, a tubular mesh with quadrilateral openings can be made by 3D printing with an elastomeric polymer. In an example, a tubular mesh with quadrilateral openings can be made by 3D printing with a silicone-based polymer. In an example, a tubular mesh with quadrilateral openings can be made by 3D printing with polydimethylsiloxane (PDMS).

In an example, a tubular mesh can have circular openings In an example, a tubular mesh with circular openings can be made using 3D printing. In an example, a flexible metal tubular mesh with circular openings can be made by 3D printing with liquid metal. In an example, a tubular mesh with circular openings can be made by 3D printing with a polymer. In an example, a tubular mesh with circular openings can be made by 3D printing with an elastomeric polymer. In an example, a tubular mesh with circular openings can be made by 3D printing with a silicone-based polymer. In an example, a tubular mesh with circular openings can be made by 3D printing with polydimethylsiloxane (PDMS).

In an example, a tubular mesh can be made with a cobalt chromium alloy. In an example, a tubular mesh can be made with a nickel-titanium alloy. In an example, a tubular mesh can comprise cobalt chromium alloy wires, filaments, or tubes. In an example, a tubular mesh can comprise nickel-titanium alloy wires, filaments, or tubes. In an example, a tubular mesh can comprise nitinol wires, filaments, or tubes. In an example, a tubular mesh can be made with nitinol. In an example, a tubular mesh can comprise platinum wires, filaments, or tubes. In an example, a tubular mesh can be made with platinum. In an example, a tubular mesh can comprise stainless steel wires, filaments, or tubes. In an example, a tubular mesh can be made with stainless steel. In an example, a tubular mesh can comprise tantalum wires, filaments, or tubes. In an example, a tubular mesh can be made with tantalum.

In an example, a mid-section of tubular mesh can be more flexible than the proximal and distal sections of the tubular mesh to predispose the tubular mesh to fold over itself at that mid-section to more easily form a dual-layer flexible net or mesh via inversion or eversion of the tubular mesh. In an example, a mid-section of tubular mesh can be more flexible than the proximal and distal sections of the tubular mesh to predispose the tubular mesh to fold over itself at that mid-section to more easily form a dual-layer bowl-shaped flexible net or mesh via inversion or eversion of the tubular mesh. In an example, a mid-section of tubular mesh can be less dense than the proximal and distal sections of the tubular mesh to predispose the tubular mesh to fold over itself at that mid-section to more easily form a dual-layer flexible net or mesh via inversion or eversion of the tubular mesh.

In an example, a mid-section of tubular mesh can be less dense than the proximal and distal sections of the tubular mesh to predispose the tubular mesh to fold over itself at that mid-section to more easily form a dual-layer bowl-shaped flexible net or mesh via inversion or eversion of the tubular mesh. In an example, a mid-section of tubular mesh can be made with a lower-durometer material than the proximal and distal sections of the tubular mesh to predispose the tubular mesh to fold over itself at that mid-section to more easily form a dual-layer flexible net or mesh via inversion or eversion of the tubular mesh. In an example, a mid-section of tubular mesh can be made with a lower-durometer material than the proximal and distal sections of the tubular mesh to predispose the tubular mesh to fold over itself at that mid-section to more easily form a dual-layer bowl-shaped flexible net or mesh via inversion or eversion of the tubular mesh.

In an example, a distal portion (e.g. the distal half) of a flexible net or mesh can have a lower durometer than the proximal portion (e.g. the proximal half) of the flexible net or mesh. In an example, a distal portion (e.g. the distal half) of a flexible net or mesh can be more flexible than the proximal portion (e.g. the proximal half) of the flexible net or mesh. In an example, a distal portion (e.g. the distal half) of a flexible net or mesh can be less dense than the proximal portion (e.g. the proximal half) of the flexible net or mesh. In an example, a distal portion (e.g. the distal half) of a flexible net or mesh can be more porous than the proximal portion (e.g. the proximal half) of the flexible net or mesh.

In an example, a flexible net or mesh can be folded and/or compressed as it is delivered through a catheter to an aneurysm sac. In an example, a flexible net or mesh can have radial folds as it is delivered through a catheter to an aneurysm sac. In an example, a flexible net or mesh can have longitudinal folds as it is delivered through a catheter to an aneurysm sac. In an example, a flexible net or mesh can have cross-sectional folds as it is delivered through a catheter to an aneurysm sac.

In an example, a flexible net or mesh can have a longitudinal axis which spans in a proximal-to-distal direction. Proximal can be defined as being closer to the point of entry into a person's body during delivery through the person's vasculature (in the catheter) to the aneurysm and closer to the aneurysm neck after insertion into the aneurysm sac. In this example, a tubular mesh is transformed into a single-layer ellipsoidal and/or generally globular flexible net or mesh by two annular members which radially-constrain the proximal and distal ends of the tubular mesh. In this example, both of these radially-constrained ends can be inverted to project into the interior of flexible net or mesh. In this example, the proximal end can be inverted to project into the interior of flexible net or mesh and the distal end can remain outside the interior of the flexible net or mesh. In this example, a tubular mesh is transformed into single-layer spherical flexible net or mesh by two annular members which radially-constrain the proximal and distal ends of the tubular mesh.

In an example, bound and/or inverted ends of a flexible net or mesh can both extend into the interior of a flexible net or mesh in a spherical, ellipsoidal, and/or globular configuration. In an example, a distal bound and/or inverted end of a flexible net or mesh can extend into the interior of a flexible net or mesh in a spherical, ellipsoidal, and/or globular configuration and a proximal bound and/or inverted end of the flexible net or mesh can extend outward from a flexible net or mesh in a spherical, ellipsoidal, and/or globular configuration. In an example, a proximal bound and/or inverted end of a flexible net or mesh can extend into the interior of a flexible net or mesh in a spherical, ellipsoidal, and/or globular configuration and a distal bound and/or inverted end of the flexible net or mesh can extend outward from a flexible net or mesh in a spherical, ellipsoidal, and/or globular configuration.

In an example, a tubular mesh can be transformed into a single-layer, distally-concave, bowl-shaped flexible net or mesh by a single annular member which radially-constrains the proximal end of the tubular mesh. In an example, a tubular mesh can be transformed into single-layer, proximally-concave, bowl-shaped flexible net or mesh by a single annular member which radially-constrains the distal end of the tubular mesh.

In an example, a tubular mesh can be transformed into a double-layer, distally-concave, bowl-shaped flexible net or mesh by two annular members which radially-constrain the proximal and distal ends of the tubular mesh, wherein the distal portion of the tubular mesh is inverted proximally (e.g. folded proximally) until it has a distally-concave shape. In this example, the distal circumference of the flexible net or mesh is a fold in the net or mesh. In an example, both of the radially-constrained ends can project into the interior of flexible net or mesh. In an example, the proximal end can be inverted to project into the interior of bowl-shaped flexible net or mesh and the distal end is not. Alternatively, a tubular mesh can be transformed into double-layer, distally-concave, bowl-shaped flexible net or mesh by a single annular member in a middle section (between the ends) of the tubular mesh which radially-constrains the middle of the tubular mesh, wherein the proximal portion of the tubular mesh is everted distally until it has a distally-concave shape. In an example, the distal circumference of the flexible net or mesh comprises two nested tubular openings.

In an example, a tubular mesh can be made from polycarbonate urethane (PCU). In an example, a tubular mesh can be made from polydimethylsiloxane (PDMS). In an example, a tubular mesh can be made from polyesters. In an example, a tubular mesh can be made from polyether block amide (PEBA). In an example, a tubular mesh can be made from polyetherether ketone (PEEK). In an example, a tubular mesh can be made from polyethylene. In an example, a tubular mesh can be made from polyethylene glycol (PEG). In an example, a tubular mesh can be made from polyethylene terephthalate (PET).

In an example, a tubular mesh can be made from polyglycolic acid (PGA). In an example, a tubular mesh can be made from polylactic acid (PLA). In an example, a tubular mesh can be made from poly-N-acetylglucosamine (PNAG). In an example, a tubular mesh can be made from polyolefin. In an example, a tubular mesh can be made from polyoleandlena. In an example, a tubular mesh can be made from polypropylene. In an example, a tubular mesh can be made from polytetrafluoroethylene (PTFE). In an example, a tubular mesh can be made from polyurethane (PU). In an example, a tubular mesh can be made from polywanacrakor. In an example, a tubular mesh can be made from polyvinyl alcohol (PVA). In an example, a tubular mesh can be made from polyvinyl pyrrolidone (PVP).

In an example, a tubular mesh from which a flexible net or mesh is formed can be tapered. In an example, the distal end of a tubular mesh can have a smaller diameter than the proximal end of the tubular mesh. In an example, the distal end of a tubular mesh can have a larger diameter than the proximal end of the tubular mesh. In an example, a tubular mesh from which a flexible net or mesh is formed can have differential flexibility. In an example the distal portion of a tubular mesh can have a first level of flexibility and the proximal portion of the tubular mesh can have a second level of flexibility, wherein the first level is less than the second level. In an example the distal portion of a tubular mesh can have a first level of flexibility and the proximal portion of the tubular mesh can have a second level of flexibility, wherein the first level is greater than the second level.

In an example, a tubular mesh from which a flexible net or mesh is formed can have differential porosity. In an example the distal portion of a tubular mesh can have a first porosity level and the proximal portion of the tubular mesh can have a second porosity level, wherein the first level is less than the second level. In an example the distal portion of a tubular mesh can have a first porosity level and the proximal portion of the tubular mesh can have a second porosity level, wherein the first level is greater than the second level. In an example, a tubular mesh from which a flexible net or mesh is formed can have differential durometer. In an example the distal portion of a tubular mesh can have a first durometer level and the proximal portion of the tubular mesh can have a second durometer level, wherein the first level is less than the second level. In an example the distal portion of a tubular mesh can have a first durometer level and the proximal portion of the tubular mesh can have a second durometer level, wherein the first level is greater than the second level.

In an example, the width of a flexible net or mesh in a bowl-shaped configuration can be larger than the width of the aneurysm neck. In an example, the circumference of a flexible net or mesh in a bowl-shaped configuration can be larger than the circumference of the aneurysm neck. In an example, the width of a flexible net or mesh in a bowl-shaped configuration can be at least 10% larger than the width of the aneurysm neck. In an example, the circumference of a flexible net or mesh in a bowl-shaped configuration can be at least 10% larger than the circumference of the aneurysm neck. In an example, the width of a flexible net or mesh in a bowl-shaped configuration can be at least 90% of the maximum width of the aneurysm sac (parallel to the aneurysm neck). In an example, the circumference of a flexible net or mesh in a bowl-shaped configuration can be at least 90% of the circumference of the maximum circumference of the aneurysm sac (parallel to the aneurysm neck). In an example, a flexible net or mesh can function as a neck bridge, reducing or completely blocking blood flow from the parent vessel into the aneurysm sac.

In an example, a flexible net or mesh formed from a tubular mesh can have a generally-hemispherical shape after a tubular mesh has been radially-constrained by one or more annular members. In an example, a flexible net or mesh formed from a tubular mesh can have a generally globular and/or spherical shape after a tubular mesh has been radially-constrained by one or more annular members. In an example, a flexible net or mesh formed from a tubular mesh can have an ellipsoidal or oval shape after a tubular mesh has been radially-constrained by one or more annular members. In an example, a flexible net or mesh formed from a tubular mesh can have a disk shape after a tubular mesh has been radially-constrained by one or more annular members.

In an example, a flexible net or mesh formed from a tubular mesh can have the shape of a paraboloid-of-revolution (e.g. a paraboloid revolved around a left or right vertical edge) after a tubular mesh has been radially-constrained by one or more annular members. In an example, a flexible net or mesh formed from a tubular mesh can comprise a carlavian curve shape after a tubular mesh has been radially-constrained by one or more annular members. In an example, a flexible net or mesh formed from a tubular mesh can have a toroidal shape after a tubular mesh has been radially-constrained by one or more annular members. In an example, a flexible net or mesh formed from a tubular mesh can have a half-toroidal shape (e.g. a sliced bagel shape) after a tubular mesh has been radially-constrained by one or more annular members.

In an example, the distal end of a tubular mesh can be radially-constrained by a distal annular member and the proximal end of a tubular mesh can be radially-constrained by a proximal annular member to form a generally-globular, spherical, and/or ellipsoidal flexible net or mesh which is inserted into an aneurysm sac. In an example, the distal end of a tubular mesh can be radially-constrained by a distal annular member and the proximal end of a tubular mesh can be radially-constrained by a proximal annular member to form a generally-globular, spherical, and/or ellipsoidal shape, wherein the distal portion is then inverted and/or folded to create a two-layer bowl-shaped flexible net or mesh which is inserted into an aneurysm sac. In an example, both the distal end of a tubular mesh and the proximal end of a tubular mesh can be radially-constrained by a proximal annular member to form a two-layer bowl-shaped flexible net or mesh which is inserted into an aneurysm sac.

In an example a flexible net or mesh can be a two-layer bowl-shaped mesh with a distally-concave proximal layer and a distally-concave distal layer. In an example a flexible net or mesh can be a two-layer bowl-shaped mesh with a distally-concave proximal layer and a distally-concave distal layer, wherein the distance between the proximal and distal layers is greater in a radially-central portion of the flexible net or mesh than in radially-peripheral portions of the flexible net or mesh. In an example a flexible net or mesh can be a two-layer bowl-shaped mesh with a proximal layer and a distal layer, wherein the proximal layer has a uniform distal-facing concavity, but the distal layer has locally-concave and locally-convex portions. In an example, the radially-central portion of the distal layer is locally-convex and the radially-peripheral portions of the distal layer are locally-concave. In an example, the radially-central portion of the distal layer is less distally-concave than the radially-peripheral portions of the distal layer.

In an example, embolic members and/or material which is inserted into the flexible net or mesh can be microspheres or microballs. In an example, embolic members and/or material inserted into the flexible net or mesh can be microsponges. In an example, embolic members and/or material inserted into the flexible net or mesh can be pieces of foam. In an example, embolic members and/or material inserted into the flexible net or mesh can be microbeads. In an example, embolic members and/or material inserted into the flexible net or mesh can be pieces of hydrogel. In an example, embolic members and/or material inserted into the flexible net or mesh can be metal embolic coils. In an example, embolic members and/or material inserted into the flexible net or mesh can be embolic ribbons. In an example, embolic members and/or material inserted into the flexible net or mesh can be yarns or filaments. In an example, embolic members and/or material can be polymer strands or coils. In an example, accumulation of embolic members and/or material in an aneurysm sac can compress a flexible net or mesh from a spherical, ellipsoidal, and/or globular configuration into a hemispherical, bowl-shaped, and/or distally-concave configuration by pressing against the distal surface of the flexible net or mesh.

In an example, embolic members and/or material inserted into the flexible net or mesh can be microspheres or microballs connected by a longitudinal wire, cord, and/or filament (e.g. in a “string-of-pearls” configuration). In an example, embolic members and/or material inserted into the flexible net or mesh can be microsponges connected by a longitudinal wire, cord, and/or filament (e.g. in a “string-of-pearls” configuration). In an example, embolic members and/or material inserted into the flexible net or mesh can be pieces of foam connected by a longitudinal wire, cord, and/or filament (e.g. in a “string-of-pearls” configuration). In an example, embolic members and/or material inserted into the flexible net or mesh can be microbeads connected by a longitudinal wire, cord, and/or filament (e.g. in a “string-of-pearls” configuration).

In an example, embolic members and/or material inserted into the flexible net or mesh can be pieces of hydrogel connected by a longitudinal wire, cord, and/or filament (e.g. in a “string-of-pearls” configuration). In an example, embolic members and/or material inserted into the flexible net or mesh can be embolic coils connected by a longitudinal wire, cord, and/or filament (e.g. in a “string-of-pearls” configuration). In an example, embolic members and/or material inserted into the flexible net or mesh can be embolic ribbons connected by a longitudinal wire, cord, and/or filament (e.g. in a “string-of-pearls” configuration). In an example, embolic members and/or material inserted into the flexible net or mesh can be yarns or filaments connected by a longitudinal wire, cord, and/or filament (e.g. in a “string-of-pearls” configuration).

In an example, embolic members and/or material inserted into the flexible net or mesh can be liquid which congeals and/or solidifies. In an example, embolic members and/or material inserted into the flexible net or mesh can be a polymer which congeals and/or solidifies. In an example, embolic members and/or material inserted into the flexible net or mesh can be a liquid embolic material. In an example, embolic members and/or material inserted into the flexible net or mesh can be hydrogel material. In an example, embolic members and/or material inserted into the flexible net or mesh can be congealing adhesive material. In an example, accumulation of embolic members and/or material in an aneurysm sac can compress a flexible net or mesh from a spherical, ellipsoidal, and/or globular configuration to a hemispherical, bowl-shaped, and/or distally-concave configuration by pressing against the distal surface of the flexible net or mesh.

In an example, embolic members and/or material which is inserted through an annular member into a flexible net or mesh can be one or more mesh ribbons. In an example, embolic members and/or material which is inserted through an annular member into a flexible net or mesh can be one or more wire mesh ribbons. In an example, embolic members and/or material which is inserted through an annular member into a flexible net or mesh can be one or more polymer mesh ribbons. In an example, embolic members and/or material which is inserted through an annular member into a flexible net or mesh can be one or more undulating and/or sinusoidal ribbons. In an example, embolic members and/or material which is inserted through an annular member into a flexible net or mesh can be one or more double-layer mesh ribbons.

In an example, embolic members and/or material can be made with a cobalt chromium alloy. In an example, embolic members and/or material can be made with a nickel-titanium alloy. In an example, embolic members and/or material can be cobalt chromium alloy coils or ribbons. In an example, embolic members and/or material can be nickel-titanium alloy coils or ribbons. In an example, embolic members and/or material can be nitinol coils or ribbons. In an example, embolic members and/or material can be made with nitinol. In an example, embolic members and/or material can be platinum coils or ribbons. In an example, embolic members and/or material can be made with platinum. In an example, embolic members and/or material can be stainless steel coils or ribbons. In an example, embolic members and/or material can be made with stainless steel. In an example, embolic members and/or material can be tantalum coils or ribbons. In an example, embolic members and/or material can be made with tantalum.

In an example, embolic members and/or material can be pushed through a catheter into a flexible net or mesh by a pusher wire and/or plug. In an example, liquid embolic material (which congeals after insertion into the net or mesh) can be pushed through a catheter into a flexible net or mesh by fluid pressure. In an example, embolic members can be pushed into a flexible net or mesh by a flow of liquid (e.g. saline solution), wherein the embolic members are retained in the flexible net or mesh and the saline solution escapes out of openings in the flexible net or mesh. In an example, embolic members and/or material can be pushed through a catheter into a flexible net or mesh by a conveyer belt mechanism. In an example, embolic members and/or material can be pushed through a catheter into a flexible net or mesh by a rotating helical delivery mechanism.

In an example, embolic members which are inserted into a net or mesh can be embolic coils or ribbons. In an example, embolic members which are inserted into a net or mesh can be pieces of foam or gel (such as hydrogel). In an example, embolic members which are inserted into a net or mesh can be microballs or microspheres. In an example, embolic members which are inserted into a net or mesh can be microsponges. In an example, embolic members which are inserted into a net or mesh can be filaments or yarns. In an example, liquid embolic material can be inserted into a net or mesh.

In an example, embolic members which are inserted into a net or mesh can be selected from the group consisting of: pieces of gel; pieces of foam; and micro-sponges. In an example, embolic members which are inserted into a net or mesh can be pieces of gel, such as hydrogel. In an example, embolic members which are inserted into a net or mesh can be pieces of foam. In an example, embolic members which are inserted into a net or mesh can be micro-sponges. In an example, embolic members which are inserted into a net or mesh can be microscale gel balls. In an example, embolic members which are inserted into a net or mesh can be microscale foam balls. In an example, embolic members which are inserted into a net or mesh can be microscale sponge balls. In an example, embolic members which are inserted into a net or mesh can be microscale gel polyhedrons. In an example, embolic members which are inserted into a net or mesh can be microscale foam polyhedrons. In an example, embolic members which are inserted into a net or mesh can be microscale sponge polyhedrons.

In an example, embolic members which are inserted into a net or mesh can have generally spherical or globular shapes. In an example, embolic members which are inserted into a net or mesh can have generally prolate spherical, ellipsoidal, or ovaloid shapes. In an example, embolic members which are inserted into a net or mesh can have apple, barrel, or pair shapes. In an example, embolic members which are inserted into a net or mesh can have torus or ring shapes. In an example, embolic members which are inserted into a net or mesh can have disk or pancake shapes. In an example, embolic members which are inserted into a net or mesh can have peanut or hour-glass shapes. In an example, embolic members which are inserted into a net or mesh can be polyhedrons comprised of hexagonal surfaces. In an example, embolic members which are inserted into a net or mesh can be polyhedrons comprised of quadrilateral surfaces. In an example, embolic members which are inserted into a net or mesh can be polyhedrons comprised of triangular surfaces.

In an example, an embolic member can have a shape which is selected from the group consisting of: apple-shaped, barrel-shaped, bulbous, convex, ellipsoidal, globular, oblate spheroid, ovaloid, prolate-spheroid-shaped, spherical, and truncated-sphere-shaped. In an example, an embolic member can have a shape which is selected from the group consisting of: bowl-shaped, concave, hemispherical, and paraboloid of revolution. In an example, an embolic member can have a shape which is selected from the group consisting of: cubic, hexagon-shaped, hexahedron, octagon-shaped, octahedron, pentagonal-shaped, polyhedron-shaped, pyramidal, rectangular, square, and tetrahedronal.

In an example, embolic members which are inserted into a net or mesh can have a (diameter) size within the range of 0.5 to 2 millimeters. In an example, embolic members which are inserted into a net or mesh can have a (diameter) size within the range of 1 to 5 millimeters. In an example, embolic members which are inserted into a net or mesh can have a (diameter) size within the range of 2 to 10 millimeters. In an example, embolic members which are inserted into a net or mesh can have a (diameter) size within the range of 5 to 20 millimeters. In an example, embolic members which are inserted into a net or mesh can have a (diameter) size within the range of 0.5 to 2 microns. In an example, embolic members which are inserted into a net or mesh can have a (diameter) size within the range of 1 to 5 microns. In an example, embolic members which are inserted into a net or mesh can have a (diameter) size within the range of 2 to 10 microns. In an example, embolic members which are inserted into a net or mesh can have a (diameter) size within the range of 5 to 20 microns.

In an example, between 5 and 20 embolic members can be inserted into a net or mesh. In an example, between 10 and 50 embolic members can be inserted into a net or mesh. In an example, between 20 and 100 embolic members can be inserted into a net or mesh. In an example, between 50 and 500 embolic members can be inserted into a net or mesh.

In an example, embolic members which are inserted into a net or mesh can expand in size within the net or mesh. In an example, embolic members can have a first (average) size while being delivered to an aneurysm sac via a micro-catheter and a second (average) size after expansion within the aneurysm sac, wherein the second (average) size is 10% to 50% larger than the first (average) size. In an example, embolic members can have a first (average) size while being delivered to an aneurysm sac via a micro-catheter and a second (average) size after expansion within the aneurysm sac, wherein the second (average) size is 40% to 100% larger than the first (average) size. In an example, embolic members can have a first (average) size while being delivered to an aneurysm sac via a micro-catheter and a second (average) size after expansion within the aneurysm sac, wherein the second (average) size is more than twice the first (average) size.

In an example, embolic members can self-expand within a net or mesh after they are released from a delivery catheter. In an example, embolic members can swell upon hydration from interaction with blood or other body fluid. In an example, embolic members can be expanded within the net or mesh by one or more mechanisms selected from the group consisting of: expansion due to interaction with body fluid; expansion due to application of thermal energy; expansion due to exposure to a chemical agent; and expansion due to exposure to light energy. In an example, embolics can expand by a factor of 2-5 times. In an example, embolics can expand by a factor of 4-10 times. In an example, embolics can expand by a factor of more than 10 times. In an example, embolic members can expand to a sufficiently-large size that they cannot escape from the net or mesh after insertion into the net or mesh.

In an example, three-dimensional embolic members which are inserted into a net or mesh can be soft and compressible. In an example, three-dimensional embolic members which are inserted into a net or mesh can have a durometer less than 50. In an example, three-dimensional embolic members which are inserted into a net or mesh can have an average durometer within the range of 10 to 30. In an example, three-dimensional embolic members which are inserted into a net or mesh can have an average durometer within the range of 25 to 50. In an example, three-dimensional embolic members which are inserted into a net or mesh can have an average durometer which is less than 70.

In an example, embolic members which are inserted into a net or mesh can be made from a polymer. In an example, embolic members which are inserted into a net or mesh can be made from an elastomeric polymer. In an example, embolic members which are inserted into a net or mesh can be made from a silicone-based polymer. In an example, embolic members which are inserted into a net or mesh can be made from polydimethylsiloxane (PDMS).

In an example, an embolic member can further comprise one or more layers made with different materials. In an example, an inner layer of an embolic member can be made from a first material and an outer layer of an embolic member can be made from a second material. In an example, an inner layer of an embolic member can be made from a first material with a first durometer and an outer layer of an embolic member can be made from a second material with a second durometer, wherein the second durometer is less than the first durometer. In an example, an embolic member can have an outer layer which is adhesive. In an example, an embolic member can have an outer layer with an adhesive property which is activated by application of electromagnetic and/or thermal energy. In an example, an embolic member can have an outer layer with an adhesive property which is activated by interaction with blood.

In an example, there can be a first average durometer of embolic members which are inserted into the net or mesh at a first time and a second average durometer of embolic members which are inserted into the net or mesh at a second time, wherein the second average durometer is greater than the first average durometer. In an example, there can be a first average durometer of embolic members which are inserted into the net or mesh at a first time and a second average durometer of embolic members which are inserted into the net or mesh at a second time, wherein the second average durometer is less than the first average durometer.

In an example, there can be a first average length of longitudinal strands between proximal pairs of embolic members which are inserted into a net or mesh at a first time, a second average length of longitudinal strands between proximal pairs of embolic members which are inserted into the net or mesh at a second time, and the second average length can be greater than the first average length. In an example, there can be a first average length of longitudinal strands between proximal pairs of embolic members which are inserted into a net or mesh at a first time, a second average length of longitudinal strands between proximal pairs of embolic members which are inserted into the net or mesh at a second time, and the second average length can be less than the first average length.

In an example, there can be a first set of embolic members which are inserted into a net or mesh at a first time and a second set of embolic members which are inserted into the net or mesh at a second time, wherein the second set of embolic members are closer together than the first set of embolic members. In an example, there can be a first set of embolic members which are inserted into a net or mesh at a first time and a second set of embolic members which are inserted into the net or mesh at a second time, wherein the first set of embolic members are closer together than the second set of embolic members. In an example, there can be a longitudinal series of embolic members connected by one or more longitudinal strands which is inserted into a net or mesh within an aneurysm sac, wherein embolic members in the longitudinal series are progressively closer to each other moving along the length of the series in a distal to proximal manner. In an example, there can be a longitudinal series of embolic members connected by one or more longitudinal strands which is inserted into a net or mesh within an aneurysm sac, wherein embolic members in the longitudinal series are progressively farther from each other moving along the length of the series in a distal to proximal manner.

In an example, embolic members which are inserted into the net or mesh at a first time can have first shapes, embolic members which are inserted into the net or mesh at a second time can have second shapes, and the second shape can be different than the first shape. In an example, embolic members which are inserted into the net or mesh at a first time can be made with a first (combination of) material, embolic members which are inserted into the net or mesh at a second time can be made with a second (combination of) material, and the second (combination of) material can be different from the first (combination of) material. In an example, embolic members which are inserted into the net or mesh at a first time can be made with a first (combination of) material, embolic members which are inserted into the net or mesh at a second time can be made with a second (combination of) material, and the second (combination of) material can be more flexible, elastic, and/or compliant than the first (combination of) material.

In an example, embolic members which are inserted into the net or mesh at a first time can be made with a first (combination of) material, embolic members which are inserted into the net or mesh at a second time can be made with a second (combination of) material, and the second (combination of) material can have a lower durometer than the first (combination of) material. In an example, embolic members which are inserted into the net or mesh at a first time can be made with a first (combination of) material, embolic members which are inserted into the net or mesh at a second time can be made with a second (combination of) material, and the second (combination of) material can be less flexible, elastic, and/or compliant than the first (combination of) material. In an example, embolic members which are inserted into the net or mesh at a first time can be made with a first (combination of) material, embolic members which are inserted into the net or mesh at a second time can be made with a second (combination of) material, and the second (combination of) material can have a higher durometer than the first (combination of) material.

In an example, there can be a first average size of embolic members which are inserted into the net or mesh at a first time, a second average size of embolic members which are inserted into the net or mesh at a second time, and the second average size can be greater than the first average size. In an example, there can be a first average size of embolic members which are inserted into the net or mesh at a first time, a second average size of embolic members which are inserted into the net or mesh at a second time, and the second average size can be less than the first average size.

In an example, a net or mesh can be delivered into an aneurysm sac via a catheter and/or delivery tube. In an example, a plurality of embolic members can be delivered into the net or mesh via the same catheter and/or delivery tube. In an example, a net or mesh can be delivered into an aneurysm sac via a first catheter and/or delivery tube and a plurality of embolic members can be delivered into the net or mesh via a second catheter and/or delivery tube.

In an example, embolic members can be made from ethylene vinyl alcohol (EVA). In an example, embolic members can be made from polyolefin. In an example, embolic members can be made from fibrinogen. In an example, embolic members can be made from polylactic acid (PLA). In an example, embolic members can be made from polyethylene terephthalate (PET). In an example, embolic members can be made from steel (e.g. stainless steel). In an example, embolic members can be made from methylcellulose.

In an example, embolic members can be made from acrylic. In an example, embolic members can be made from polyethylene glycol (PEG). In an example, embolic members can be made from silk. In an example, embolic members can be made from alginate. In an example, embolic members can be made from gold. In an example, embolic members can be made from polyethylene. In an example, embolic members can be made from polyoleandlena. In an example, embolic members can be made from tantalum. In an example, embolic members can be made from cobalt-chrome alloy (cobalt chromium).

In an example, embolic members can be made from polyetherether ketone (PEEK). In an example, embolic members can be made from polywanacrakor. In an example, embolic members can be made from thermoplastic elastomer. In an example, embolic members can be made from polycarbonate urethane (PCU). In an example, embolic members can be made from water-soluble synthetic polymer. In an example, embolic members can be made from collagen. In an example, embolic members can be made from polyvinyl alcohol (PVA).

In an example, embolic members can be made from titanium. In an example, embolic members can be made from polyether block amide (PEBA). In an example, embolic members can be made from radiopaque material. In an example, embolic members can be made from copolymer. In an example, embolic members can be made from polyvinyl pyrrolidone (PVP). In an example, embolic members can be made from polydimethylsiloxane (PDMS). In an example, embolic members can be made from zirconium-based alloy. In an example, embolic members can be made from polyesters. In an example, embolic members can be made from hydrogel. In an example, embolic members can be made from silicone. In an example, embolic members can be made from nitinol (or other nickel titanium alloy).

In an example, embolic members can be made from polyglycolic acid (PGA). In an example, embolic members can be made from small intestinal submucosa. In an example, embolic members can be made from nylon. In an example, embolic members can be made from polypropylene. In an example, embolic members can be made from platinum. In an example, embolic members can be made from polyurethane (PU). In an example, embolic members can be made from tungsten. In an example, embolic members can be made from fibrin.

In an example, embolic members can be made from poly-N-acetylglucosamine (PNAG). In an example, embolic members can be made from latex. In an example, embolic members can be made from fibronectin. In an example, embolic members can be made from palladium. In an example, embolic members can be made from polytetrafluoroethylene (PTFE). In an example, embolic members can be made from gelatin.

In an example, a selected quantity, series, length, and/or volume of embolic members can be selectively dispensed and/or detached into the net or mesh in situ by a mechanism selected from the group consisting of: breaking a connection between embolic members in a series of embolic members; cutting a connection between embolic members in a series of embolic members (e.g. with a cutting edge or laser); dissolving a connection between embolic members in a series of embolic members (e.g. with thermal energy or a chemical); electrolytic mechanism; hydraulic mechanism; injecting a flow of embolic members suspended in a liquid or gel into a net or mesh; melting a connection between embolic members in a series of embolic members (e.g. with thermal or light energy); progressing embolic members into a net or mesh via a conveyor belt (e.g. chain-based conveyor); progressing embolic members into a net or mesh via a helical conveyor (e.g. with an Archimedes' screw); pushing embolic members into a net or mesh using the force of a liquid flow; pusher rod and/or plunger; release detachment mechanism; and thermal detachment mechanism.

In an example, embolic members can differ among themselves with respect to one or more characteristics selected from the group consisting of: porosity, shape, size, material, composition, coating, radiopacity, strength, stiffness, and type. In an example, a plurality of embolic members can be delivered into a net or mesh in a linear (longitudinal) array or series of inter-connected embolic members. In an example, a plurality of embolic members can be delivered into a net or mesh in a linear (longitudinal) array of connected embolic members, wherein this linear array can be cut, separated, and/or detached in situ (in a remote manner) at one or more selected locations by the user of the device in order to deliver a selected quantity, length, or volume or embolic members. In an example, a plurality of embolic members can be delivered into a net or mesh in a planar array of inter-connected embolic members. In an example, a plurality of embolic members can be delivered into a net or mesh in a three-dimensional array of inter-connected embolic members.

In an example, a series of embolic members can be delivered into a net or mesh, wherein successive embolic members in the series are closer together. In an example, a series of embolic members can be delivered into a net or mesh, wherein embolic members in the series are progressively closer together (as one progresses along the series in a distal to proximal manner). In an example, a series of embolic members can be delivered into a net or mesh, wherein successive embolic members in the series are farther apart from each other. In an example, a series of embolic members can be delivered into a net or mesh, wherein embolic members in the series are progressively farther apart (as one progresses along the series in a distal to proximal manner).

In an example, a series of embolic members can be delivered into a net or mesh, wherein successive embolic members in the series decrease in durometer. In an example, a series of embolic members can be delivered into a net or mesh, wherein embolic members in the series have progressively lower durometer values (as one progresses along the series in a distal to proximal manner). In an example, a series of embolic members can be delivered into a net or mesh, wherein successive embolic members in the series increase in durometer. In an example, a series of embolic members can be delivered into a net or mesh, wherein embolic members in the series have progressively higher durometer values (as one progresses along the series in a distal to proximal manner).

In an example, a series of embolic members can be delivered into a net or mesh, wherein successive embolic members in the series are made of different materials. In an example, a series of embolic members can be delivered into a net or mesh, wherein successive embolic members in the series are made of different materials, wherein these materials differ in porosity. In an example, a series of embolic members can be delivered into a net or mesh, wherein successive embolic members in the series are made of different materials, wherein these materials differ in radiopacity. In an example, a series of embolic members can be delivered into a net or mesh, wherein successive embolic members in the series are made of different materials, wherein these materials differ in stiffness. In an example, a series of embolic members can be delivered into a net or mesh, wherein successive embolic members in the series are made of different materials, wherein these materials differ in durometer.

In an example, a series of embolic members can be delivered into a net or mesh, wherein successive embolic members in the series decrease in porosity. In an example, a series of embolic members can be delivered into a net or mesh, wherein embolic members in the series become progressively less porous (as one progresses along the series in a distal to proximal manner). In an example, a series of embolic members can be delivered into a net or mesh, wherein successive embolic members in the series increase in porosity. In an example, a series of embolic members can be delivered into a net or mesh, wherein embolic members in the series become progressively more porous (as one progresses along the series in a distal to proximal manner).

In an example, a series of embolic members can be delivered into a net or mesh, wherein successive embolic members in the series differ in shape. In an example, a series of embolic members can be delivered into a net or mesh, wherein successive embolic members in the series differ in their degree of convexity. In an example, a series of embolic members can be delivered into a net or mesh, wherein successive embolic members in the series differ in their degree of concavity.

In an example, a series of embolic members can be delivered into a net or mesh, wherein successive embolic members in the series decrease in size. In an example, a series of embolic members can be delivered into a net or mesh, wherein embolic members in the series become progressively smaller (as one progresses along the series in a distal to proximal manner). In an example, a series of embolic members can be delivered into a net or mesh, wherein successive embolic members in the series increase in size. In an example, a series of embolic members can be delivered into a net or mesh, wherein embolic members in the series become progressively larger (as one progresses along the series in a distal to proximal manner).

In an example, embolic members can be soft, compressible members such as microsponges or blobs of gel. In an example, embolic members can be made from sponge, foam, or gel. In an example, embolic members can be hard, uncompressible members such as hard polymer spheres or beads. In an example, embolic members can be made from one or more materials selected from the group consisting of: cellulose, collagen, acetate, alginic acid, carboxy methyl cellulose, chitin, collagen glycosaminoglycan, divinylbenzene, ethylene glycol, ethylene glycol dimethylmathacrylate, ethylene vinyl acetate, hyaluronic acid, hydrocarbon polymer, hydroxyethylmethacrylate, methlymethacrylate, polyacrylic acid, polyamides, polyesters, polyolefins, polysaccharides, polyurethane, polyvinyl alcohol, silicone, urethane, and vinyl stearate.

In an example, embolic members can have a shape selected from the group consisting of: ball or sphere, ovoid, ellipsoid, and polyhedron. In an example, embolic members can have a Shore OO value, indicative of softness or hardness, within a range of 5 to about 50. In an example, embolic members can have a diameter or like size within a range of 50 micrometers to 2000 micrometers. In an example, differently-sized embolic members can be used. In an example two or more different sizes of embolic members can be inserted into a net or mesh to occlude an aneurysm. In an example, embolic members can include small balls and large balls. In an example, it may be advantageous to first fill a net or mesh with larger balls and then continue filling the net or mesh with smaller balls. In another example, it may be advantageous to first fill a net or mesh with smaller balls and then continue filling the net or mesh with larger balls.

In an example, an intrasaccular aneurysm occlusion device can be filled with a “string of pearls” string (or wire) connected sequence of embolic members. In an example, an intrasaccular aneurysm occlusion device can include a series of embolic members which are connected by a strand. In an example, a device can include a string of pearls” series of embolic members which are linked by a strand (e.g. a thin flexible member). In an example, a device can include a string of pearls” series of embolic members which are centrally linked by a strand (e.g. a thin flexible member). In an example, a “string of pearls” string-or-wire connected sequence of embolic members can comprise a plurality of embolic members which are separate from each other, but pair-wise connected to each other by at least one string or wire. In an example, a plurality of members can be unevenly-spaced along the longitudinal axis of a flexible member. In an example, uneven spacing of the embolic members can be selected based on the size and shape of an aneurysm to be occluded. In an example, the distances between embolic members can vary. In an example, the space between embolic members can differ for occlusion of narrow-neck aneurysms vs. wide-neck aneurysms. In an example, distances between embolic members can become progressively shorter in a distal to proximal direction.

In an example, a line which connects embolic members can be a wire, spring, or chain. In an example, a connecting line can be a string, thread, band, fiber, or suture. In an example, embolic members can be centrally connected to each other by a connecting line. In an example, the centroids of embolic members can be connected by a connecting line. In an example, expanding arcuate embolic members can slide (e.g. up or down) along a connecting line. In an example, embolic members can slide along a connecting line, but only in one direction. In an example, a connecting line can have a ratchet structure which allows embolic members to slide closer to each other but not slide further apart. In an example, this device can further comprise a locking mechanism which stops embolic members from sliding along a connecting line. In an example, application of electromagnetic energy to a connecting line can fuse the line with the embolic members and stop them from sliding, effectively locking them in proximity to each other.

In an example, embolic members can be conveyed through a lumen to an aneurysm in a fluid flow, wherein the fluid escapes out from a net or mesh and the embolic members are retained within the net or mesh. In an example, embolic members can be conveyed through a lumen to an aneurysm by means of a moving belt or wire loop. In an example, embolic members can be conveyed through a lumen to an aneurysm by means of an Archimedes screw.

In an example, a flexible net or mesh can self-expand to a first extent after being released from a catheter into an aneurysm sac. In an example, the flexible net or mesh can further expand, to a second extent, due to pressure from the accumulation of embolic members and/or embolic material within its interior and/or distal-facing concavity. In an example, a flexible net or mesh can further expand to conform to the wall contours of even an irregularly-shaped aneurysm sac. Other example variations discussed elsewhere in this disclosure or in priority-linked disclosures can also be applied to this example where relevant.

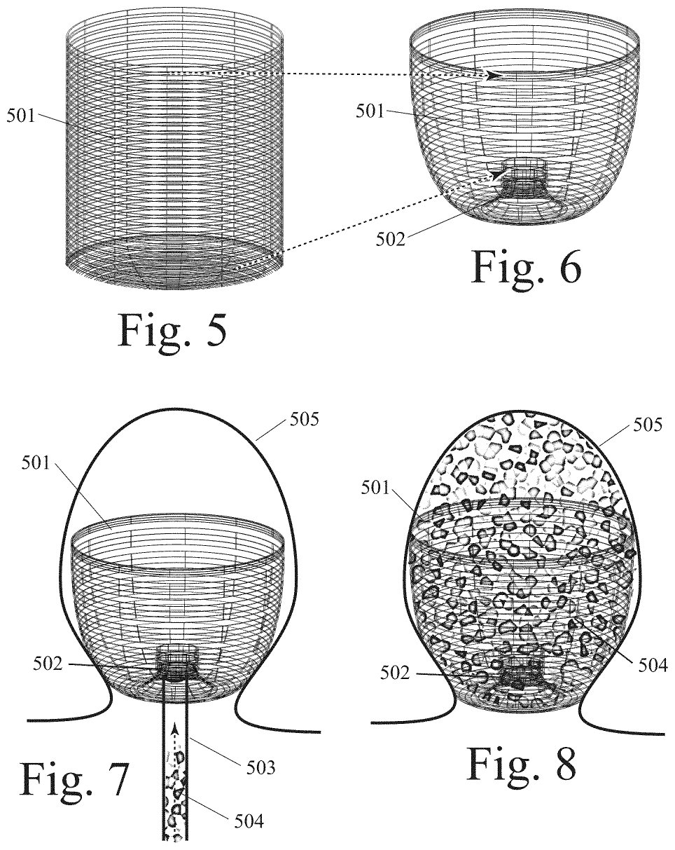

FIGS. 5 through 8 show four views, at different times, of the formation and deployment of another example of an intrasacular aneurysm occlusion device comprising: at least one annular member (in this example, proximal annular member 502), wherein an annular member is selected from the group consisting of a ring, a band, a cylinder, a tube, and a catheter; a flexible net or mesh, wherein the flexible net or mesh has a spherical, ellipsoidal, generally-globular, hemispherical, and/or bowl-shaped first configuration when it is formed by encircling, pinching, inverting, and/or everting a tubular mesh 501 at one or more longitudinal locations using the at least one annular member; wherein the flexible net or mesh has a radially-compressed second configuration for delivery through a catheter 503 into an aneurysm sac 505; and wherein the flexible net or mesh is inserted and expanded within the aneurysm sac; and embolic members and/or embolic material 504 which is inserted into the interior and/or the distal-facing concavity of the flexible net or mesh through one or more of the annular members. In this example, there is one annular member—a proximal annular member which radially constrains the proximal end of the tubular mesh. In this example, the flexible net or mesh has a single-layer bowl shape when it is first formed from the tubular mesh.

FIG. 5 introduces tubular mesh 501 which is used to make the intrasacular aneurysm occlusion device. FIG. 6 shows a proximal annular member (ring or band in this example) 502 which radially-constrains the proximal end of the tubular mesh, changing the tubular mesh into a single-layer bowl-shaped flexible net or mesh. In this example, the proximal end of the tubular mesh is inverted into the distal-facing concavity of the bowl-shaped flexible net or mesh. FIG. 7 shows the flexible net or mesh after it has been radially-compressed for delivery through a catheter 503 into an aneurysm sac 505, the flexible net or mesh has been inserted into the aneurysm sac, and embolic members and/or material 504 is starting to be delivered through the catheter (and through the proximal annular member 502) into the distal-facing concavity of the flexible net or mesh. FIG. 8 shows the distal portion of aneurysm sac having been filed with embolic members and/or material and the catheter having been removed.

In this example, an annular member is a ring or band which encircles a middle portion (between the ends) of the tubular mesh. In an example, an annular member can be a metal ring, band, or cylinder. In an example, an annular member can be a polymer ring, band, or cylinder. In an example, an annular member can be a wire, cord, or string. In an example, an annular member can be a ring or band which encircles a tubular mesh, thereby radially-constraining and/or pinching the tubular mesh but allowing embolic members and/or embolic material to pass through it into the interior and/or a concavity of the flexible net or mesh. In an example, an annular member can be a cylinder which encircles a tubular mesh, thereby radially-constraining and/or pinching the tubular mesh but allowing embolic members and/or embolic material to pass through it into the interior and/or a concavity of the flexible net or mesh.

In an example, an annular member can be a cord or wire which encircles a tubular mesh, thereby radially-constraining and/or pinching the tubular mesh but allowing embolic members and/or embolic material to pass through it into the interior and/or a concavity of the flexible net or mesh. In an example, an annular member can be a catheter or tube around which a tubular mesh is attached, thereby radially-constraining and/or pinching the tubular mesh but allowing embolic members and/or embolic material to pass through it into the interior and/or a concavity of the flexible net or mesh. In an example, an annular member can be a lumen through a flexible net or mesh through which embolic members and/or material is inserted into the flexible net or mesh.

In an example, a tubular mesh can be soldered, melted, glued, or crimped onto an annular member. In an example, an annular member can have an inner ring and an outer ring, wherein a tubular mesh is fixed (e.g. soldered, melted, glued, or crimped) between the two rings. In an example, an annular member can comprise an inner ring or cylinder and an outer elastic band, wherein the tubular mesh is held between the inner and outer portions. In this example, an annular member can be centrally-located with respect to a proximal surface of the flexible net or mesh. In an example, an annular member can be centrally-located with respect to the longitudinal axis of the flexible net or mesh. In an example, an annular member can be a hub into which proximal ends of braided wires or tubes of the stent are bound or attached. In an example, an annular member can be off-axial with respect to the longitudinal axis of the flexible net or mesh.

In an example, an annular member can comprise two nested and/or concentric (inner and outer) cylinders, wherein the tubular mesh is pinched and/or crimped between the two cylinders. In an example, an annular member can comprise two nested and/or concentric (inner and outer) rings or bands, wherein the tubular mesh is pinched and/or crimped between the two rings or bands. In an example, an annular member can comprise two nested and/or concentric (inner and outer) cylinders, wherein the tubular mesh is melted or glued between the two cylinders. In an example, an annular member can comprise two nested and/or concentric (inner and outer) rings or bands, wherein the tubular mesh is melted or glued between the two rings or bands.

In an example, an annular member can be a catheter which extends through the proximal surface of a flexible net or mesh, wherein the catheter is detached and/or removed after embolic members and/or material has been inserted through the catheter into the interior or distal-facing concavity of the flexible net or mesh. In an example, a distal portion of the catheter used to deliver embolic members and/or material can extend through the proximal surface of a flexible net or mesh and be detached from the rest of the catheter after embolic members and/or material has been inserted through the catheter. In an example, an annular member can be attached to a catheter during delivery of embolic members and/or material, and then detached (e.g. by the application of electromagnetic energy) from the catheter after delivery of the embolic members and/or material.

In an example, an annular member can have an outer diameter which is between 5% and 20% of the diameter of the tubular mesh before the tubular mesh is radially constrained. In an example, an annular member can have an outer diameter which is between 10% and 33% of the diameter of the tubular mesh before the tubular mesh is radially constrained. In an example, an annular member can have an outer ring (or cylinder) with a first diameter and an inner ring (or cylinder) with a second diameter, wherein the tubular mesh is crimped or pinched between the outer ring (or cylinder) and inner ring (or cylinder), and wherein the first diameter is between 50% and 75% of the second diameter. In an example, an annular member can have an outer ring (or cylinder) with a first diameter and an inner ring (or cylinder) with a second diameter, wherein the tubular mesh is crimped or pinched between the outer ring (or cylinder) and inner ring (or cylinder), and wherein the first diameter is between 66% and 90% of the second diameter.

In an example, an annular member can comprise two nested rings, bands, or cylinders, wherein a section of the tubular mesh is inserted and held between the nested rings, bands, or cylinders. In an example, an annular member can comprise an outer ring, band, or cylinder and an inner ring, band, or cylinder, wherein a section of the tubular mesh is inserted and held between them. In an example, an annular member can comprise an outer ring, band, or cylinder and an inner ring, band, or cylinder, wherein one or both of the rings, bands, or cylinders are threaded. In an example, an annular member can comprise an outer ring, band, or cylinder and an inner ring, band, or cylinder, wherein one or both of the rings, bands, or cylinders has a helical thread. In an example, an annular member can comprise an outer ring, band, or cylinder and an inner ring, band, or cylinder, wherein one or both of the rings, bands, or cylinders has a helical thread to hold a section of the tubular mesh.

In an example, this device can further comprise a closure mechanism which closes an opening through an annular member. The closure mechanism can be closed after embolic members and/or material has been inserted into a flexible net or mesh. In an example, this closure mechanism can be selected from the group consisting of: valve; electric detachment mechanism; elastic ring or band; threaded mechanism; sliding cover; sliding plug; filament loop; and electromagnetic solenoid. In an example, a closure mechanism can be a leaflet valve. In an example, a closure mechanism can be a one-way valve. In an example, a valve can allow embolic members and/or material to enter a flexible net or mesh through an opening in an annular member, but not allow the embolic members and/or material to exit the net or mesh.

In an example, a tubular mesh can be made from a polymer. In an example, a tubular mesh can be woven or braided from polymer threads, filaments, yarns, or strips. In an example, a tubular mesh can be 3D printed. In an example, a flexible net or mesh can be made from a flexible polymer. In an example, a flexible net or mesh can be made from an elastic and/or stretchable polymer. In an example, a flexible net or mesh can be elastic and/or stretchable and can expand as it is filled with embolic members and/or material. In an example, a flexible net or mesh can be sufficiently flexible to conform to the shape of even an irregularly-shaped aneurysm sac as the net or mesh is filled with embolic members and/or material. In an example, a flexible net or mesh can be sufficiently flexible to conform to the shape of even an irregularly-shaped (e.g. non-spherical) aneurysm sac as the net or mesh is filled with embolic members and/or material. In an example, a tubular mesh can be made from one or more materials selected from the group consisting of: Dacron, elastin, hydroxy-terminated polycarbonate, methylcellulose, nylon, PDMS, polybutester, polycaprolactone, polyester, polyethylene terephthalate, polypropylene, polytetrafluoroethene, polytetrafluoroethylene, polyurethane, silicone, and silk.

In an example, a tubular mesh can be made from metal. In an example, a tubular mesh can be made from Nitinol. In an example, a tubular mesh can be a flexible metal mesh. In an example, a tubular mesh can be a braided metal mesh. In an example, a tubular mesh can be woven or braided from metal filaments, wires, or tubes. In an example, a tubular mesh can be made from shape-memory material. In an example, a tubular mesh can be made with both metal and polymer components.

In an example, openings or holes in a flexible net or mesh can be smaller than the size (e.g. diameter, width, and/or length) of embolic members and/or material which is inserted into the net or mesh so that the embolic members and/or material do not escape out of the net or mesh. In an example, openings or holes in a flexible net or mesh can less than half of the size (e.g. diameter, width, and/or length) of embolic members and/or material which is inserted into the net or mesh so that the embolic members and/or material do not escape out of the net or mesh. In an example, openings or holes in a flexible net or mesh can have a size which is less than half of the smallest diameter and/or width of embolic members and/or material which is inserted into the net or mesh so that the embolic members and/or material do not escape out of the net or mesh. In an example, openings or holes in a flexible net or mesh can have a size which less than half of the smallest length of embolic members and/or material which is inserted into the net or mesh so that the embolic members and/or material do not escape out of the net or mesh.

In an example, a tubular mesh can have hexagonal openings In an example, a tubular mesh with hexagonal openings can be made using 3D printing. In an example, a flexible metal tubular mesh with hexagonal openings can be made by 3D printing with liquid metal. In an example, a tubular mesh with hexagonal openings can be made by 3D printing with a polymer. In an example, a tubular mesh with hexagonal openings can be made by 3D printing with an elastomeric polymer. In an example, a tubular mesh with hexagonal openings can be made by 3D printing with a silicone-based polymer. In an example, a tubular mesh with hexagonal openings can be made by 3D printing with polydimethylsiloxane (PDMS).

In an example, a tubular mesh can have quadrilateral openings. In an example, a tubular mesh with quadrilateral openings can be made using 3D printing. In an example, a flexible metal tubular mesh with quadrilateral openings can be made by 3D printing with liquid metal. In an example, a tubular mesh with quadrilateral openings can be made by 3D printing with a polymer. In an example, a tubular mesh with quadrilateral openings can be made by 3D printing with an elastomeric polymer. In an example, a tubular mesh with quadrilateral openings can be made by 3D printing with a silicone-based polymer. In an example, a tubular mesh with quadrilateral openings can be made by 3D printing with polydimethylsiloxane (PDMS).

In an example, a tubular mesh can have circular openings In an example, a tubular mesh with circular openings can be made using 3D printing. In an example, a flexible metal tubular mesh with circular openings can be made by 3D printing with liquid metal. In an example, a tubular mesh with circular openings can be made by 3D printing with a polymer. In an example, a tubular mesh with circular openings can be made by 3D printing with an elastomeric polymer. In an example, a tubular mesh with circular openings can be made by 3D printing with a silicone-based polymer. In an example, a tubular mesh with circular openings can be made by 3D printing with polydimethylsiloxane (PDMS).

In an example, a tubular mesh can be made with a cobalt chromium alloy. In an example, a tubular mesh can be made with a nickel-titanium alloy. In an example, a tubular mesh can comprise cobalt chromium alloy wires, filaments, or tubes. In an example, a tubular mesh can comprise nickel-titanium alloy wires, filaments, or tubes. In an example, a tubular mesh can comprise nitinol wires, filaments, or tubes. In an example, a tubular mesh can be made with nitinol. In an example, a tubular mesh can comprise platinum wires, filaments, or tubes. In an example, a tubular mesh can be made with platinum. In an example, a tubular mesh can comprise stainless steel wires, filaments, or tubes. In an example, a tubular mesh can be made with stainless steel. In an example, a tubular mesh can comprise tantalum wires, filaments, or tubes. In an example, a tubular mesh can be made with tantalum.

In an example, a mid-section of tubular mesh can be more flexible than the proximal and distal sections of the tubular mesh to predispose the tubular mesh to fold over itself at that mid-section to more easily form a dual-layer flexible net or mesh via inversion or eversion of the tubular mesh. In an example, a mid-section of tubular mesh can be more flexible than the proximal and distal sections of the tubular mesh to predispose the tubular mesh to fold over itself at that mid-section to more easily form a dual-layer bowl-shaped flexible net or mesh via inversion or eversion of the tubular mesh. In an example, a mid-section of tubular mesh can be less dense than the proximal and distal sections of the tubular mesh to predispose the tubular mesh to fold over itself at that mid-section to more easily form a dual-layer flexible net or mesh via inversion or eversion of the tubular mesh.

In an example, a mid-section of tubular mesh can be less dense than the proximal and distal sections of the tubular mesh to predispose the tubular mesh to fold over itself at that mid-section to more easily form a dual-layer bowl-shaped flexible net or mesh via inversion or eversion of the tubular mesh. In an example, a mid-section of tubular mesh can be made with a lower-durometer material than the proximal and distal sections of the tubular mesh to predispose the tubular mesh to fold over itself at that mid-section to more easily form a dual-layer flexible net or mesh via inversion or eversion of the tubular mesh. In an example, a mid-section of tubular mesh can be made with a lower-durometer material than the proximal and distal sections of the tubular mesh to predispose the tubular mesh to fold over itself at that mid-section to more easily form a dual-layer bowl-shaped flexible net or mesh via inversion or eversion of the tubular mesh.

In an example, a distal portion (e.g. the distal half) of a flexible net or mesh can have a lower durometer than the proximal portion (e.g. the proximal half) of the flexible net or mesh. In an example, a distal portion (e.g. the distal half) of a flexible net or mesh can be more flexible than the proximal portion (e.g. the proximal half) of the flexible net or mesh. In an example, a distal portion (e.g. the distal half) of a flexible net or mesh can be less dense than the proximal portion (e.g. the proximal half) of the flexible net or mesh. In an example, a distal portion (e.g. the distal half) of a flexible net or mesh can be more porous than the proximal portion (e.g. the proximal half) of the flexible net or mesh.

In an example, a flexible net or mesh can be folded and/or compressed as it is delivered through a catheter to an aneurysm sac. In an example, a flexible net or mesh can have radial folds as it is delivered through a catheter to an aneurysm sac. In an example, a flexible net or mesh can have longitudinal folds as it is delivered through a catheter to an aneurysm sac. In an example, a flexible net or mesh can have cross-sectional folds as it is delivered through a catheter to an aneurysm sac.

In an example, a flexible net or mesh can have a longitudinal axis which spans in a proximal-to-distal direction. Proximal can be defined as being closer to the point of entry into a person's body during delivery through the person's vasculature (in the catheter) to the aneurysm and closer to the aneurysm neck after insertion into the aneurysm sac. In this example, a tubular mesh is transformed into a single-layer, distally-concave, bowl-shaped flexible net or mesh by a single annular member which radially-constrains the proximal end of the tubular mesh. In an example, a tubular mesh can be transformed into single-layer, proximally-concave, bowl-shaped flexible net or mesh by a single annular member which radially-constrains the distal end of the tubular mesh.

In an example, a tubular mesh can be transformed into a single-layer ellipsoidal and/or generally globular flexible net or mesh by two annular members which radially-constrain the proximal and distal ends of the tubular mesh. In an example, both of these radially-constrained ends can be inverted to project into the interior of flexible net or mesh. In an example, the proximal end can be inverted to project into the interior of flexible net or mesh and the distal end can remain outside the interior of the flexible net or mesh. In an example, a tubular mesh is transformed into single-layer spherical flexible net or mesh by two annular members which radially-constrain the proximal and distal ends of the tubular mesh.

In an example, bound and/or inverted ends of a flexible net or mesh can both extend into the interior of a flexible net or mesh in a spherical, ellipsoidal, and/or globular configuration. In an example, a distal bound and/or inverted end of a flexible net or mesh can extend into the interior of a flexible net or mesh in a spherical, ellipsoidal, and/or globular configuration and a proximal bound and/or inverted end of the flexible net or mesh can extend outward from a flexible net or mesh in a spherical, ellipsoidal, and/or globular configuration. In an example, a proximal bound and/or inverted end of a flexible net or mesh can extend into the interior of a flexible net or mesh in a spherical, ellipsoidal, and/or globular configuration and a distal bound and/or inverted end of the flexible net or mesh can extend outward from a flexible net or mesh in a spherical, ellipsoidal, and/or globular configuration.

In an example, a tubular mesh can be transformed into a double-layer, distally-concave, bowl-shaped flexible net or mesh by two annular members which radially-constrain the proximal and distal ends of the tubular mesh, wherein the distal portion of the tubular mesh is inverted proximally (e.g. folded proximally) until it has a distally-concave shape. In this example, the distal circumference of the flexible net or mesh is a fold in the net or mesh. In an example, both of the radially-constrained ends can project into the interior of flexible net or mesh. In an example, the proximal end can be inverted to project into the interior of bowl-shaped flexible net or mesh and the distal end is not. Alternatively, a tubular mesh can be transformed into double-layer, distally-concave, bowl-shaped flexible net or mesh by a single annular member in a middle section (between the ends) of the tubular mesh which radially-constrains the middle of the tubular mesh, wherein the proximal portion of the tubular mesh is everted distally until it has a distally-concave shape. In an example, the distal circumference of the flexible net or mesh comprises two nested tubular openings.

In an example, a tubular mesh can be made from polycarbonate urethane (PCU). In an example, a tubular mesh can be made from polydimethylsiloxane (PDMS). In an example, a tubular mesh can be made from polyesters. In an example, a tubular mesh can be made from polyether block amide (PEBA). In an example, a tubular mesh can be made from polyetherether ketone (PEEK). In an example, a tubular mesh can be made from polyethylene. In an example, a tubular mesh can be made from polyethylene glycol (PEG). In an example, a tubular mesh can be made from polyethylene terephthalate (PET).

In an example, a tubular mesh can be made from polyglycolic acid (PGA). In an example, a tubular mesh can be made from polylactic acid (PLA). In an example, a tubular mesh can be made from poly-N-acetylglucosamine (PNAG). In an example, a tubular mesh can be made from polyolefin. In an example, a tubular mesh can be made from polyoleandlena. In an example, a tubular mesh can be made from polypropylene. In an example, a tubular mesh can be made from polytetrafluoroethylene (PTFE). In an example, a tubular mesh can be made from polyurethane (PU). In an example, a tubular mesh can be made from polywanacrakor. In an example, a tubular mesh can be made from polyvinyl alcohol (PVA). In an example, a tubular mesh can be made from polyvinyl pyrrolidone (PVP).

In an example, a tubular mesh from which a flexible net or mesh is formed can be tapered. In an example, the distal end of a tubular mesh can have a smaller diameter than the proximal end of the tubular mesh. In an example, the distal end of a tubular mesh can have a larger diameter than the proximal end of the tubular mesh. In an example, a tubular mesh from which a flexible net or mesh is formed can have differential flexibility. In an example the distal portion of a tubular mesh can have a first level of flexibility and the proximal portion of the tubular mesh can have a second level of flexibility, wherein the first level is less than the second level. In an example the distal portion of a tubular mesh can have a first level of flexibility and the proximal portion of the tubular mesh can have a second level of flexibility, wherein the first level is greater than the second level.

In an example, a tubular mesh from which a flexible net or mesh is formed can have differential porosity. In an example the distal portion of a tubular mesh can have a first porosity level and the proximal portion of the tubular mesh can have a second porosity level, wherein the first level is less than the second level. In an example the distal portion of a tubular mesh can have a first porosity level and the proximal portion of the tubular mesh can have a second porosity level, wherein the first level is greater than the second level. In an example, a tubular mesh from which a flexible net or mesh is formed can have differential durometer. In an example the distal portion of a tubular mesh can have a first durometer level and the proximal portion of the tubular mesh can have a second durometer level, wherein the first level is less than the second level. In an example the distal portion of a tubular mesh can have a first durometer level and the proximal portion of the tubular mesh can have a second durometer level, wherein the first level is greater than the second level.

In an example, the width of a flexible net or mesh in a bowl-shaped configuration can be larger than the width of the aneurysm neck. In an example, the circumference of a flexible net or mesh in a bowl-shaped configuration can be larger than the circumference of the aneurysm neck. In an example, the width of a flexible net or mesh in a bowl-shaped configuration can be at least 10% larger than the width of the aneurysm neck. In an example, the circumference of a flexible net or mesh in a bowl-shaped configuration can be at least 10% larger than the circumference of the aneurysm neck. In an example, the width of a flexible net or mesh in a bowl-shaped configuration can be at least 90% of the maximum width of the aneurysm sac (parallel to the aneurysm neck). In an example, the circumference of a flexible net or mesh in a bowl-shaped configuration can be at least 90% of the circumference of the maximum circumference of the aneurysm sac (parallel to the aneurysm neck). In an example, a flexible net or mesh can function as a neck bridge, reducing or completely blocking blood flow from the parent vessel into the aneurysm sac.

In an example, a flexible net or mesh formed from a tubular mesh can have a generally-hemispherical shape after a tubular mesh has been radially-constrained by one or more annular members. In an example, a flexible net or mesh formed from a tubular mesh can have a generally globular and/or spherical shape after a tubular mesh has been radially-constrained by one or more annular members. In an example, a flexible net or mesh formed from a tubular mesh can have an ellipsoidal or oval shape after a tubular mesh has been radially-constrained by one or more annular members. In an example, a flexible net or mesh formed from a tubular mesh can have a disk shape after a tubular mesh has been radially-constrained by one or more annular members.

In an example, a flexible net or mesh formed from a tubular mesh can have the shape of a paraboloid-of-revolution (e.g. a paraboloid revolved around a left or right vertical edge) after a tubular mesh has been radially-constrained by one or more annular members. In an example, a flexible net or mesh formed from a tubular mesh can comprise a carlavian curve shape after a tubular mesh has been radially-constrained by one or more annular members. In an example, a flexible net or mesh formed from a tubular mesh can have a toroidal shape after a tubular mesh has been radially-constrained by one or more annular members. In an example, a flexible net or mesh formed from a tubular mesh can have a half-toroidal shape (e.g. a sliced bagel shape) after a tubular mesh has been radially-constrained by one or more annular members.

In an example, the distal end of a tubular mesh can be radially-constrained by a distal annular member and the proximal end of a tubular mesh can be radially-constrained by a proximal annular member to form a generally-globular, spherical, and/or ellipsoidal flexible net or mesh which is inserted into an aneurysm sac. In an example, the distal end of a tubular mesh can be radially-constrained by a distal annular member and the proximal end of a tubular mesh can be radially-constrained by a proximal annular member to form a generally-globular, spherical, and/or ellipsoidal shape, wherein the distal portion is then inverted and/or folded to create a two-layer bowl-shaped flexible net or mesh which is inserted into an aneurysm sac. In an example, both the distal end of a tubular mesh and the proximal end of a tubular mesh can be radially-constrained by a proximal annular member to form a two-layer bowl-shaped flexible net or mesh which is inserted into an aneurysm sac.

In an example a flexible net or mesh can be a two-layer bowl-shaped mesh with a distally-concave proximal layer and a distally-concave distal layer. In an example a flexible net or mesh can be a two-layer bowl-shaped mesh with a distally-concave proximal layer and a distally-concave distal layer, wherein the distance between the proximal and distal layers is greater in a radially-central portion of the flexible net or mesh than in radially-peripheral portions of the flexible net or mesh. In an example a flexible net or mesh can be a two-layer bowl-shaped mesh with a proximal layer and a distal layer, wherein the proximal layer has a uniform distal-facing concavity, but the distal layer has locally-concave and locally-convex portions. In an example, the radially-central portion of the distal layer is locally-convex and the radially-peripheral portions of the distal layer are locally-concave. In an example, the radially-central portion of the distal layer is less distally-concave than the radially-peripheral portions of the distal layer.

In an example, embolic members and/or material which is inserted into the flexible net or mesh can be microspheres or microballs. In an example, embolic members and/or material inserted into the flexible net or mesh can be microsponges. In an example, embolic members and/or material inserted into the flexible net or mesh can be pieces of foam. In an example, embolic members and/or material inserted into the flexible net or mesh can be microbeads. In an example, embolic members and/or material inserted into the flexible net or mesh can be pieces of hydrogel. In an example, embolic members and/or material inserted into the flexible net or mesh can be metal embolic coils. In an example, embolic members and/or material inserted into the flexible net or mesh can be embolic ribbons. In an example, embolic members and/or material inserted into the flexible net or mesh can be yarns or filaments. In an example, embolic members and/or material can be polymer strands or coils. In an example, accumulation of embolic members and/or material in an aneurysm sac can compress a flexible net or mesh from a spherical, ellipsoidal, and/or globular configuration into a hemispherical, bowl-shaped, and/or distally-concave configuration by pressing against the distal surface of the flexible net or mesh.

In an example, embolic members and/or material inserted into the flexible net or mesh can be microspheres or microballs connected by a longitudinal wire, cord, and/or filament (e.g. in a “string-of-pearls” configuration). In an example, embolic members and/or material inserted into the flexible net or mesh can be microsponges connected by a longitudinal wire, cord, and/or filament (e.g. in a “string-of-pearls” configuration). In an example, embolic members and/or material inserted into the flexible net or mesh can be pieces of foam connected by a longitudinal wire, cord, and/or filament (e.g. in a “string-of-pearls” configuration). In an example, embolic members and/or material inserted into the flexible net or mesh can be microbeads connected by a longitudinal wire, cord, and/or filament (e.g. in a “string-of-pearls” configuration).

In an example, embolic members and/or material inserted into the flexible net or mesh can be pieces of hydrogel connected by a longitudinal wire, cord, and/or filament (e.g. in a “string-of-pearls” configuration). In an example, embolic members and/or material inserted into the flexible net or mesh can be embolic coils connected by a longitudinal wire, cord, and/or filament (e.g. in a “string-of-pearls” configuration). In an example, embolic members and/or material inserted into the flexible net or mesh can be embolic ribbons connected by a longitudinal wire, cord, and/or filament (e.g. in a “string-of-pearls” configuration). In an example, embolic members and/or material inserted into the flexible net or mesh can be yarns or filaments connected by a longitudinal wire, cord, and/or filament (e.g. in a “string-of-pearls” configuration).

In an example, embolic members and/or material inserted into the flexible net or mesh can be liquid which congeals and/or solidifies. In an example, embolic members and/or material inserted into the flexible net or mesh can be a polymer which congeals and/or solidifies. In an example, embolic members and/or material inserted into the flexible net or mesh can be a liquid embolic material. In an example, embolic members and/or material inserted into the flexible net or mesh can be hydrogel material. In an example, embolic members and/or material inserted into the flexible net or mesh can be congealing adhesive material. In an example, accumulation of embolic members and/or material in an aneurysm sac can compress a flexible net or mesh from a spherical, ellipsoidal, and/or globular configuration to a hemispherical, bowl-shaped, and/or distally-concave configuration by pressing against the distal surface of the flexible net or mesh.

In an example, embolic members and/or material which is inserted through an annular member into a flexible net or mesh can be one or more mesh ribbons. In an example, embolic members and/or material which is inserted through an annular member into a flexible net or mesh can be one or more wire mesh ribbons. In an example, embolic members and/or material which is inserted through an annular member into a flexible net or mesh can be one or more polymer mesh ribbons. In an example, embolic members and/or material which is inserted through an annular member into a flexible net or mesh can be one or more undulating and/or sinusoidal ribbons. In an example, embolic members and/or material which is inserted through an annular member into a flexible net or mesh can be one or more double-layer mesh ribbons.

In an example, embolic members and/or material can be made with a cobalt chromium alloy. In an example, embolic members and/or material can be made with a nickel-titanium alloy. In an example, embolic members and/or material can be cobalt chromium alloy coils or ribbons. In an example, embolic members and/or material can be nickel-titanium alloy coils or ribbons. In an example, embolic members and/or material can be nitinol coils or ribbons. In an example, embolic members and/or material can be made with nitinol. In an example, embolic members and/or material can be platinum coils or ribbons. In an example, embolic members and/or material can be made with platinum. In an example, embolic members and/or material can be stainless steel coils or ribbons. In an example, embolic members and/or material can be made with stainless steel. In an example, embolic members and/or material can be tantalum coils or ribbons. In an example, embolic members and/or material can be made with tantalum.

In an example, embolic members and/or material can be pushed through a catheter into a flexible net or mesh by a pusher wire and/or plug. In an example, liquid embolic material (which congeals after insertion into the net or mesh) can be pushed through a catheter into a flexible net or mesh by fluid pressure. In an example, embolic members can be pushed into a flexible net or mesh by a flow of liquid (e.g. saline solution), wherein the embolic members are retained in the flexible net or mesh and the saline solution escapes out of openings in the flexible net or mesh. In an example, embolic members and/or material can be pushed through a catheter into a flexible net or mesh by a conveyer belt mechanism. In an example, embolic members and/or material can be pushed through a catheter into a flexible net or mesh by a rotating helical delivery mechanism.

In an example, embolic members which are inserted into a net or mesh can be embolic coils or ribbons. In an example, embolic members which are inserted into a net or mesh can be pieces of foam or gel (such as hydrogel). In an example, embolic members which are inserted into a net or mesh can be microballs or microspheres. In an example, embolic members which are inserted into a net or mesh can be microsponges. In an example, embolic members which are inserted into a net or mesh can be filaments or yarns. In an example, liquid embolic material can be inserted into a net or mesh.

In an example, embolic members which are inserted into a net or mesh can be selected from the group consisting of: pieces of gel; pieces of foam; and micro-sponges. In an example, embolic members which are inserted into a net or mesh can be pieces of gel, such as hydrogel. In an example, embolic members which are inserted into a net or mesh can be pieces of foam. In an example, embolic members which are inserted into a net or mesh can be micro-sponges. In an example, embolic members which are inserted into a net or mesh can be microscale gel balls. In an example, embolic members which are inserted into a net or mesh can be microscale foam balls. In an example, embolic members which are inserted into a net or mesh can be microscale sponge balls. In an example, embolic members which are inserted into a net or mesh can be microscale gel polyhedrons. In an example, embolic members which are inserted into a net or mesh can be microscale foam polyhedrons. In an example, embolic members which are inserted into a net or mesh can be microscale sponge polyhedrons.

In an example, embolic members which are inserted into a net or mesh can have generally spherical or globular shapes. In an example, embolic members which are inserted into a net or mesh can have generally prolate spherical, ellipsoidal, or ovaloid shapes. In an example, embolic members which are inserted into a net or mesh can have apple, barrel, or pair shapes. In an example, embolic members which are inserted into a net or mesh can have torus or ring shapes. In an example, embolic members which are inserted into a net or mesh can have disk or pancake shapes. In an example, embolic members which are inserted into a net or mesh can have peanut or hour-glass shapes. In an example, embolic members which are inserted into a net or mesh can be polyhedrons comprised of hexagonal surfaces. In an example, embolic members which are inserted into a net or mesh can be polyhedrons comprised of quadrilateral surfaces. In an example, embolic members which are inserted into a net or mesh can be polyhedrons comprised of triangular surfaces.

In an example, an embolic member can have a shape which is selected from the group consisting of: apple-shaped, barrel-shaped, bulbous, convex, ellipsoidal, globular, oblate spheroid, ovaloid, prolate-spheroid-shaped, spherical, and truncated-sphere-shaped. In an example, an embolic member can have a shape which is selected from the group consisting of: bowl-shaped, concave, hemispherical, and paraboloid of revolution. In an example, an embolic member can have a shape which is selected from the group consisting of: cubic, hexagon-shaped, hexahedron, octagon-shaped, octahedron, pentagonal-shaped, polyhedron-shaped, pyramidal, rectangular, square, and tetrahedronal.

In an example, embolic members which are inserted into a net or mesh can have a (diameter) size within the range of 0.5 to 2 millimeters. In an example, embolic members which are inserted into a net or mesh can have a (diameter) size within the range of 1 to 5 millimeters. In an example, embolic members which are inserted into a net or mesh can have a (diameter) size within the range of 2 to 10 millimeters. In an example, embolic members which are inserted into a net or mesh can have a (diameter) size within the range of 5 to 20 millimeters. In an example, embolic members which are inserted into a net or mesh can have a (diameter) size within the range of 0.5 to 2 microns. In an example, embolic members which are inserted into a net or mesh can have a (diameter) size within the range of 1 to 5 microns. In an example, embolic members which are inserted into a net or mesh can have a (diameter) size within the range of 2 to 10 microns. In an example, embolic members which are inserted into a net or mesh can have a (diameter) size within the range of 5 to 20 microns.

In an example, between 5 and 20 embolic members can be inserted into a net or mesh. In an example, between 10 and 50 embolic members can be inserted into a net or mesh. In an example, between 20 and 100 embolic members can be inserted into a net or mesh. In an example, between 50 and 500 embolic members can be inserted into a net or mesh.

In an example, embolic members which are inserted into a net or mesh can expand in size within the net or mesh. In an example, embolic members can have a first (average) size while being delivered to an aneurysm sac via a micro-catheter and a second (average) size after expansion within the aneurysm sac, wherein the second (average) size is 10% to 50% larger than the first (average) size. In an example, embolic members can have a first (average) size while being delivered to an aneurysm sac via a micro-catheter and a second (average) size after expansion within the aneurysm sac, wherein the second (average) size is 40% to 100% larger than the first (average) size. In an example, embolic members can have a first (average) size while being delivered to an aneurysm sac via a micro-catheter and a second (average) size after expansion within the aneurysm sac, wherein the second (average) size is more than twice the first (average) size.

In an example, embolic members can self-expand within a net or mesh after they are released from a delivery catheter. In an example, embolic members can swell upon hydration from interaction with blood or other body fluid. In an example, embolic members can be expanded within the net or mesh by one or more mechanisms selected from the group consisting of: expansion due to interaction with body fluid; expansion due to application of thermal energy; expansion due to exposure to a chemical agent; and expansion due to exposure to light energy. In an example, embolics can expand by a factor of 2-5 times. In an example, embolics can expand by a factor of 4-10 times. In an example, embolics can expand by a factor of more than 10 times. In an example, embolic members can expand to a sufficiently-large size that they cannot escape from the net or mesh after insertion into the net or mesh.

In an example, three-dimensional embolic members which are inserted into a net or mesh can be soft and compressible. In an example, three-dimensional embolic members which are inserted into a net or mesh can have a durometer less than 50. In an example, three-dimensional embolic members which are inserted into a net or mesh can have an average durometer within the range of 10 to 30. In an example, three-dimensional embolic members which are inserted into a net or mesh can have an average durometer within the range of 25 to 50. In an example, three-dimensional embolic members which are inserted into a net or mesh can have an average durometer which is less than 70.

In an example, embolic members which are inserted into a net or mesh can be made from a polymer. In an example, embolic members which are inserted into a net or mesh can be made from an elastomeric polymer. In an example, embolic members which are inserted into a net or mesh can be made from a silicone-based polymer. In an example, embolic members which are inserted into a net or mesh can be made from polydimethylsiloxane (PDMS).

In an example, an embolic member can further comprise one or more layers made with different materials. In an example, an inner layer of an embolic member can be made from a first material and an outer layer of an embolic member can be made from a second material. In an example, an inner layer of an embolic member can be made from a first material with a first durometer and an outer layer of an embolic member can be made from a second material with a second durometer, wherein the second durometer is less than the first durometer. In an example, an embolic member can have an outer layer which is adhesive. In an example, an embolic member can have an outer layer with an adhesive property which is activated by application of electromagnetic and/or thermal energy. In an example, an embolic member can have an outer layer with an adhesive property which is activated by interaction with blood.

In an example, there can be a first average durometer of embolic members which are inserted into the net or mesh at a first time and a second average durometer of embolic members which are inserted into the net or mesh at a second time, wherein the second average durometer is greater than the first average durometer. In an example, there can be a first average durometer of embolic members which are inserted into the net or mesh at a first time and a second average durometer of embolic members which are inserted into the net or mesh at a second time, wherein the second average durometer is less than the first average durometer.

In an example, there can be a first average length of longitudinal strands between proximal pairs of embolic members which are inserted into a net or mesh at a first time, a second average length of longitudinal strands between proximal pairs of embolic members which are inserted into the net or mesh at a second time, and the second average length can be greater than the first average length. In an example, there can be a first average length of longitudinal strands between proximal pairs of embolic members which are inserted into a net or mesh at a first time, a second average length of longitudinal strands between proximal pairs of embolic members which are inserted into the net or mesh at a second time, and the second average length can be less than the first average length.

In an example, there can be a first set of embolic members which are inserted into a net or mesh at a first time and a second set of embolic members which are inserted into the net or mesh at a second time, wherein the second set of embolic members are closer together than the first set of embolic members. In an example, there can be a first set of embolic members which are inserted into a net or mesh at a first time and a second set of embolic members which are inserted into the net or mesh at a second time, wherein the first set of embolic members are closer together than the second set of embolic members. In an example, there can be a longitudinal series of embolic members connected by one or more longitudinal strands which is inserted into a net or mesh within an aneurysm sac, wherein embolic members in the longitudinal series are progressively closer to each other moving along the length of the series in a distal to proximal manner. In an example, there can be a longitudinal series of embolic members connected by one or more longitudinal strands which is inserted into a net or mesh within an aneurysm sac, wherein embolic members in the longitudinal series are progressively farther from each other moving along the length of the series in a distal to proximal manner.

In an example, embolic members which are inserted into the net or mesh at a first time can have first shapes, embolic members which are inserted into the net or mesh at a second time can have second shapes, and the second shape can be different than the first shape. In an example, embolic members which are inserted into the net or mesh at a first time can be made with a first (combination of) material, embolic members which are inserted into the net or mesh at a second time can be made with a second (combination of) material, and the second (combination of) material can be different from the first (combination of) material. In an example, embolic members which are inserted into the net or mesh at a first time can be made with a first (combination of) material, embolic members which are inserted into the net or mesh at a second time can be made with a second (combination of) material, and the second (combination of) material can be more flexible, elastic, and/or compliant than the first (combination of) material.

In an example, embolic members which are inserted into the net or mesh at a first time can be made with a first (combination of) material, embolic members which are inserted into the net or mesh at a second time can be made with a second (combination of) material, and the second (combination of) material can have a lower durometer than the first (combination of) material. In an example, embolic members which are inserted into the net or mesh at a first time can be made with a first (combination of) material, embolic members which are inserted into the net or mesh at a second time can be made with a second (combination of) material, and the second (combination of) material can be less flexible, elastic, and/or compliant than the first (combination of) material. In an example, embolic members which are inserted into the net or mesh at a first time can be made with a first (combination of) material, embolic members which are inserted into the net or mesh at a second time can be made with a second (combination of) material, and the second (combination of) material can have a higher durometer than the first (combination of) material.

In an example, there can be a first average size of embolic members which are inserted into the net or mesh at a first time, a second average size of embolic members which are inserted into the net or mesh at a second time, and the second average size can be greater than the first average size. In an example, there can be a first average size of embolic members which are inserted into the net or mesh at a first time, a second average size of embolic members which are inserted into the net or mesh at a second time, and the second average size can be less than the first average size.

In an example, a net or mesh can be delivered into an aneurysm sac via a catheter and/or delivery tube. In an example, a plurality of embolic members can be delivered into the net or mesh via the same catheter and/or delivery tube. In an example, a net or mesh can be delivered into an aneurysm sac via a first catheter and/or delivery tube and a plurality of embolic members can be delivered into the net or mesh via a second catheter and/or delivery tube.

In an example, embolic members can be made from ethylene vinyl alcohol (EVA). In an example, embolic members can be made from polyolefin. In an example, embolic members can be made from fibrinogen. In an example, embolic members can be made from polylactic acid (PLA). In an example, embolic members can be made from polyethylene terephthalate (PET). In an example, embolic members can be made from steel (e.g. stainless steel). In an example, embolic members can be made from methylcellulose.

In an example, embolic members can be made from acrylic. In an example, embolic members can be made from polyethylene glycol (PEG). In an example, embolic members can be made from silk. In an example, embolic members can be made from alginate. In an example, embolic members can be made from gold. In an example, embolic members can be made from polyethylene. In an example, embolic members can be made from polyoleandlena. In an example, embolic members can be made from tantalum. In an example, embolic members can be made from cobalt-chrome alloy (cobalt chromium).

In an example, embolic members can be made from polyetherether ketone (PEEK). In an example, embolic members can be made from polywanacrakor. In an example, embolic members can be made from thermoplastic elastomer. In an example, embolic members can be made from polycarbonate urethane (PCU). In an example, embolic members can be made from water-soluble synthetic polymer. In an example, embolic members can be made from collagen. In an example, embolic members can be made from polyvinyl alcohol (PVA).

In an example, embolic members can be made from titanium. In an example, embolic members can be made from polyether block amide (PEBA). In an example, embolic members can be made from radiopaque material. In an example, embolic members can be made from copolymer. In an example, embolic members can be made from polyvinyl pyrrolidone (PVP). In an example, embolic members can be made from polydimethylsiloxane (PDMS). In an example, embolic members can be made from zirconium-based alloy. In an example, embolic members can be made from polyesters. In an example, embolic members can be made from hydrogel. In an example, embolic members can be made from silicone. In an example, embolic members can be made from nitinol (or other nickel titanium alloy).

In an example, embolic members can be made from polyglycolic acid (PGA). In an example, embolic members can be made from small intestinal submucosa. In an example, embolic members can be made from nylon. In an example, embolic members can be made from polypropylene. In an example, embolic members can be made from platinum. In an example, embolic members can be made from polyurethane (PU). In an example, embolic members can be made from tungsten. In an example, embolic members can be made from fibrin.

In an example, embolic members can be made from poly-N-acetylglucosamine (PNAG). In an example, embolic members can be made from latex. In an example, embolic members can be made from fibronectin. In an example, embolic members can be made from palladium. In an example, embolic members can be made from polytetrafluoroethylene (PTFE). In an example, embolic members can be made from gelatin.

In an example, a selected quantity, series, length, and/or volume of embolic members can be selectively dispensed and/or detached into the net or mesh in situ by a mechanism selected from the group consisting of: breaking a connection between embolic members in a series of embolic members; cutting a connection between embolic members in a series of embolic members (e.g. with a cutting edge or laser); dissolving a connection between embolic members in a series of embolic members (e.g. with thermal energy or a chemical); electrolytic mechanism; hydraulic mechanism; injecting a flow of embolic members suspended in a liquid or gel into a net or mesh; melting a connection between embolic members in a series of embolic members (e.g. with thermal or light energy); progressing embolic members into a net or mesh via a conveyor belt (e.g. chain-based conveyor); progressing embolic members into a net or mesh via a helical conveyor (e.g. with an Archimedes' screw); pushing embolic members into a net or mesh using the force of a liquid flow; pusher rod and/or plunger; release detachment mechanism; and thermal detachment mechanism.

In an example, embolic members can differ among themselves with respect to one or more characteristics selected from the group consisting of: porosity, shape, size, material, composition, coating, radiopacity, strength, stiffness, and type. In an example, a plurality of embolic members can be delivered into a net or mesh in a linear (longitudinal) array or series of inter-connected embolic members. In an example, a plurality of embolic members can be delivered into a net or mesh in a linear (longitudinal) array of connected embolic members, wherein this linear array can be cut, separated, and/or detached in situ (in a remote manner) at one or more selected locations by the user of the device in order to deliver a selected quantity, length, or volume or embolic members. In an example, a plurality of embolic members can be delivered into a net or mesh in a planar array of inter-connected embolic members. In an example, a plurality of embolic members can be delivered into a net or mesh in a three-dimensional array of inter-connected embolic members.

In an example, a series of embolic members can be delivered into a net or mesh, wherein successive embolic members in the series are closer together. In an example, a series of embolic members can be delivered into a net or mesh, wherein embolic members in the series are progressively closer together (as one progresses along the series in a distal to proximal manner). In an example, a series of embolic members can be delivered into a net or mesh, wherein successive embolic members in the series are farther apart from each other. In an example, a series of embolic members can be delivered into a net or mesh, wherein embolic members in the series are progressively farther apart (as one progresses along the series in a distal to proximal manner).

In an example, a series of embolic members can be delivered into a net or mesh, wherein successive embolic members in the series decrease in durometer. In an example, a series of embolic members can be delivered into a net or mesh, wherein embolic members in the series have progressively lower durometer values (as one progresses along the series in a distal to proximal manner). In an example, a series of embolic members can be delivered into a net or mesh, wherein successive embolic members in the series increase in durometer. In an example, a series of embolic members can be delivered into a net or mesh, wherein embolic members in the series have progressively higher durometer values (as one progresses along the series in a distal to proximal manner).

In an example, a series of embolic members can be delivered into a net or mesh, wherein successive embolic members in the series are made of different materials. In an example, a series of embolic members can be delivered into a net or mesh, wherein successive embolic members in the series are made of different materials, wherein these materials differ in porosity. In an example, a series of embolic members can be delivered into a net or mesh, wherein successive embolic members in the series are made of different materials, wherein these materials differ in radiopacity. In an example, a series of embolic members can be delivered into a net or mesh, wherein successive embolic members in the series are made of different materials, wherein these materials differ in stiffness. In an example, a series of embolic members can be delivered into a net or mesh, wherein successive embolic members in the series are made of different materials, wherein these materials differ in durometer.

In an example, a series of embolic members can be delivered into a net or mesh, wherein successive embolic members in the series decrease in porosity. In an example, a series of embolic members can be delivered into a net or mesh, wherein embolic members in the series become progressively less porous (as one progresses along the series in a distal to proximal manner). In an example, a series of embolic members can be delivered into a net or mesh, wherein successive embolic members in the series increase in porosity. In an example, a series of embolic members can be delivered into a net or mesh, wherein embolic members in the series become progressively more porous (as one progresses along the series in a distal to proximal manner).

In an example, a series of embolic members can be delivered into a net or mesh, wherein successive embolic members in the series differ in shape. In an example, a series of embolic members can be delivered into a net or mesh, wherein successive embolic members in the series differ in their degree of convexity. In an example, a series of embolic members can be delivered into a net or mesh, wherein successive embolic members in the series differ in their degree of concavity.

In an example, a series of embolic members can be delivered into a net or mesh, wherein successive embolic members in the series decrease in size. In an example, a series of embolic members can be delivered into a net or mesh, wherein embolic members in the series become progressively smaller (as one progresses along the series in a distal to proximal manner). In an example, a series of embolic members can be delivered into a net or mesh, wherein successive embolic members in the series increase in size. In an example, a series of embolic members can be delivered into a net or mesh, wherein embolic members in the series become progressively larger (as one progresses along the series in a distal to proximal manner).

In an example, embolic members can be soft, compressible members such as microsponges or blobs of gel. In an example, embolic members can be made from sponge, foam, or gel. In an example, embolic members can be hard, uncompressible members such as hard polymer spheres or beads. In an example, embolic members can be made from one or more materials selected from the group consisting of: cellulose, collagen, acetate, alginic acid, carboxy methyl cellulose, chitin, collagen glycosaminoglycan, divinylbenzene, ethylene glycol, ethylene glycol dimethylmathacrylate, ethylene vinyl acetate, hyaluronic acid, hydrocarbon polymer, hydroxyethylmethacrylate, methlymethacrylate, polyacrylic acid, polyamides, polyesters, polyolefins, polysaccharides, polyurethane, polyvinyl alcohol, silicone, urethane, and vinyl stearate.

In an example, embolic members can have a shape selected from the group consisting of: ball or sphere, ovoid, ellipsoid, and polyhedron. In an example, embolic members can have a Shore OO value, indicative of softness or hardness, within a range of 5 to about 50. In an example, embolic members can have a diameter or like size within a range of 50 micrometers to 2000 micrometers. In an example, differently-sized embolic members can be used. In an example two or more different sizes of embolic members can be inserted into a net or mesh to occlude an aneurysm. In an example, embolic members can include small balls and large balls. In an example, it may be advantageous to first fill a net or mesh with larger balls and then continue filling the net or mesh with smaller balls. In another example, it may be advantageous to first fill a net or mesh with smaller balls and then continue filling the net or mesh with larger balls.

In an example, an intrasaccular aneurysm occlusion device can be filled with a “string of pearls” string (or wire) connected sequence of embolic members. In an example, an intrasaccular aneurysm occlusion device can include a series of embolic members which are connected by a strand. In an example, a device can include a string of pearls” series of embolic members which are linked by a strand (e.g. a thin flexible member). In an example, a device can include a string of pearls” series of embolic members which are centrally linked by a strand (e.g. a thin flexible member). In an example, a “string of pearls” string-or-wire connected sequence of embolic members can comprise a plurality of embolic members which are separate from each other, but pair-wise connected to each other by at least one string or wire. In an example, a plurality of members can be unevenly-spaced along the longitudinal axis of a flexible member. In an example, uneven spacing of the embolic members can be selected based on the size and shape of an aneurysm to be occluded. In an example, the distances between embolic members can vary. In an example, the space between embolic members can differ for occlusion of narrow-neck aneurysms vs. wide-neck aneurysms. In an example, distances between embolic members can become progressively shorter in a distal to proximal direction.

In an example, a line which connects embolic members can be a wire, spring, or chain. In an example, a connecting line can be a string, thread, band, fiber, or suture. In an example, embolic members can be centrally connected to each other by a connecting line. In an example, the centroids of embolic members can be connected by a connecting line. In an example, expanding arcuate embolic members can slide (e.g. up or down) along a connecting line. In an example, embolic members can slide along a connecting line, but only in one direction. In an example, a connecting line can have a ratchet structure which allows embolic members to slide closer to each other but not slide further apart. In an example, this device can further comprise a locking mechanism which stops embolic members from sliding along a connecting line. In an example, application of electromagnetic energy to a connecting line can fuse the line with the embolic members and stop them from sliding, effectively locking them in proximity to each other.

In an example, embolic members can be conveyed through a lumen to an aneurysm in a fluid flow, wherein the fluid escapes out from a net or mesh and the embolic members are retained within the net or mesh. In an example, embolic members can be conveyed through a lumen to an aneurysm by means of a moving belt or wire loop. In an example, embolic members can be conveyed through a lumen to an aneurysm by means of an Archimedes screw.

In an example, a flexible net or mesh can self-expand to a first extent after being released from a catheter into an aneurysm sac. In an example, the flexible net or mesh can further expand, to a second extent, due to pressure from the accumulation of embolic members and/or embolic material within its interior and/or distal-facing concavity. Other example variations discussed elsewhere in this disclosure or in priority-linked disclosures can also be applied to this example where relevant.

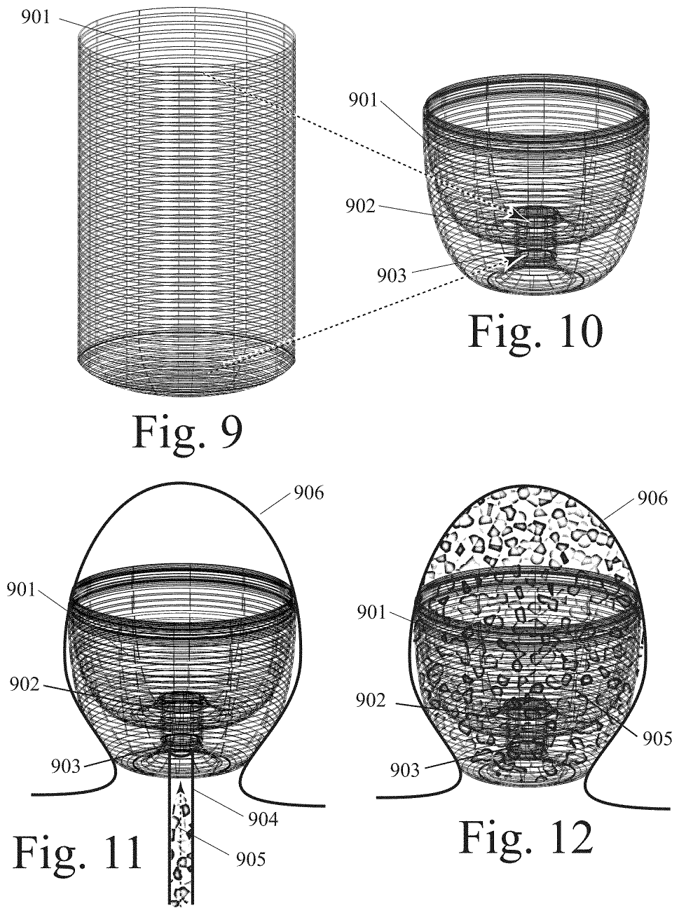

FIGS. 9 through 12 show four views, at different times, of the formation and deployment of another example of an intrasacular aneurysm occlusion device comprising: at least one annular member (two in this example, a proximal annular member 903 and a distal annular member 902), wherein an annular member is selected from the group consisting of a ring, a band, a cylinder, a tube, and a catheter; a flexible net or mesh, wherein the flexible net or mesh has a spherical, ellipsoidal, generally-globular, hemispherical, and/or bowl-shaped first configuration when it is formed by encircling, pinching, inverting, and/or everting a tubular mesh 901 at one or more longitudinal locations using the at least one annular member; wherein the flexible net or mesh has a radially-compressed second configuration for delivery through a catheter 904 into an aneurysm sac 906; and wherein the flexible net or mesh is inserted and expanded within the aneurysm sac; and embolic members and/or embolic material 905 which is inserted into the interior and/or the distal-facing concavity of the flexible net or mesh through one or more of the annular members. In this example, there are two annular members: a proximal annular member which radially constrains the proximal end of the tubular mesh; and a distal annular member which radially constrains the distal end of the tubular mesh. In this example, the flexible net or mesh has a double-layer bowl shape when it is first formed from the tubular mesh.

FIG. 9 introduces tubular mesh 901 which is used to make the intrasacular aneurysm occlusion device. FIG. 10 shows two annular members: a proximal annular member (ring or band) 903 which radially-constrains the proximal end of the tubular mesh; and a distal annular member (ring or band) 902 which radially-constrains the distal end of the tubular mesh. The two annular members transform the tubular mesh into a generally-globular mesh, which, in turn, is compressed into a double-layer bowl-shaped flexible net or mesh as shown in FIG. 10. In FIG. 10, the distal portion of the tubular mesh has been compressed and inverted into the concavity of the proximal portion of the tubular mesh, thereby forming a double-layer bowl-shaped flexible net or mesh. FIG. 11 shows the flexible net or mesh after it has been radially-compressed for delivery through a catheter 904 into an aneurysm sac 906, wherein the flexible net or mesh has been inserted into the aneurysm sac, and wherein embolic members and/or material 905 are starting to be delivered through the catheter (and through the annular members) into the distal-facing concavity of the bowl-shaped flexible net or mesh and the distal portion of the aneurysm sac. FIG. 12 shows the distal-facing concavity of the bowl-shaped flexible net or mesh and the distal portion of aneurysm sac having been filed with embolic members and/or material and the catheter having been removed.

In this example, an annular member is a ring or band which encircles a middle portion (between the ends) of the tubular mesh. In an example, an annular member can be a metal ring, band, or cylinder. In an example, an annular member can be a polymer ring, band, or cylinder. In an example, an annular member can be a wire, cord, or string. In an example, an annular member can be a ring or band which encircles a tubular mesh, thereby radially-constraining and/or pinching the tubular mesh but allowing embolic members and/or embolic material to pass through it into the interior and/or a concavity of the flexible net or mesh. In an example, an annular member can be a cylinder which encircles a tubular mesh, thereby radially-constraining and/or pinching the tubular mesh but allowing embolic members and/or embolic material to pass through it into the interior and/or a concavity of the flexible net or mesh.

In an example, an annular member can be a cord or wire which encircles a tubular mesh, thereby radially-constraining and/or pinching the tubular mesh but allowing embolic members and/or embolic material to pass through it into the interior and/or a concavity of the flexible net or mesh. In an example, an annular member can be a catheter or tube around which a tubular mesh is attached, thereby radially-constraining and/or pinching the tubular mesh but allowing embolic members and/or embolic material to pass through it into the interior and/or a concavity of the flexible net or mesh. In an example, an annular member can be a lumen through a flexible net or mesh through which embolic members and/or material is inserted into the flexible net or mesh.

In an example, a tubular mesh can be soldered, melted, glued, or crimped onto an annular member. In an example, an annular member can have an inner ring and an outer ring, wherein a tubular mesh is fixed (e.g. soldered, melted, glued, or crimped) between the two rings. In an example, an annular member can comprise an inner ring or cylinder and an outer elastic band, wherein the tubular mesh is held between the inner and outer portions. In this example, an annular member can be centrally-located with respect to a proximal surface of the flexible net or mesh. In an example, an annular member can be centrally-located with respect to the longitudinal axis of the flexible net or mesh. In an example, an annular member can be a hub into which proximal ends of braided wires or tubes of the stent are bound or attached. In an example, an annular member can be off-axial with respect to the longitudinal axis of the flexible net or mesh.

In an example, an annular member can comprise two nested and/or concentric (inner and outer) cylinders, wherein the tubular mesh is pinched and/or crimped between the two cylinders. In an example, an annular member can comprise two nested and/or concentric (inner and outer) rings or bands, wherein the tubular mesh is pinched and/or crimped between the two rings or bands. In an example, an annular member can comprise two nested and/or concentric (inner and outer) cylinders, wherein the tubular mesh is melted or glued between the two cylinders. In an example, an annular member can comprise two nested and/or concentric (inner and outer) rings or bands, wherein the tubular mesh is melted or glued between the two rings or bands.

In an example, an annular member can be a catheter which extends through the proximal surface of a flexible net or mesh, wherein the catheter is detached and/or removed after embolic members and/or material has been inserted through the catheter into the interior or distal-facing concavity of the flexible net or mesh. In an example, a distal portion of the catheter used to deliver embolic members and/or material can extend through the proximal surface of a flexible net or mesh and be detached from the rest of the catheter after embolic members and/or material has been inserted through the catheter. In an example, an annular member can be attached to a catheter during delivery of embolic members and/or material, and then detached (e.g. by the application of electromagnetic energy) from the catheter after delivery of the embolic members and/or material.

In an example, an annular member can have an outer diameter which is between 5% and 20% of the diameter of the tubular mesh before the tubular mesh is radially constrained. In an example, an annular member can have an outer diameter which is between 10% and 33% of the diameter of the tubular mesh before the tubular mesh is radially constrained. In an example, an annular member can have an outer ring (or cylinder) with a first diameter and an inner ring (or cylinder) with a second diameter, wherein the tubular mesh is crimped or pinched between the outer ring (or cylinder) and inner ring (or cylinder), and wherein the first diameter is between 50% and 75% of the second diameter. In an example, an annular member can have an outer ring (or cylinder) with a first diameter and an inner ring (or cylinder) with a second diameter, wherein the tubular mesh is crimped or pinched between the outer ring (or cylinder) and inner ring (or cylinder), and wherein the first diameter is between 66% and 90% of the second diameter.

In an example, an annular member can comprise two nested rings, bands, or cylinders, wherein a section of the tubular mesh is inserted and held between the nested rings, bands, or cylinders. In an example, an annular member can comprise an outer ring, band, or cylinder and an inner ring, band, or cylinder, wherein a section of the tubular mesh is inserted and held between them. In an example, an annular member can comprise an outer ring, band, or cylinder and an inner ring, band, or cylinder, wherein one or both of the rings, bands, or cylinders are threaded. In an example, an annular member can comprise an outer ring, band, or cylinder and an inner ring, band, or cylinder, wherein one or both of the rings, bands, or cylinders has a helical thread. In an example, an annular member can comprise an outer ring, band, or cylinder and an inner ring, band, or cylinder, wherein one or both of the rings, bands, or cylinders has a helical thread to hold a section of the tubular mesh.

In an example, this device can further comprise a closure mechanism which closes an opening through an annular member. The closure mechanism can be closed after embolic members and/or material has been inserted into a flexible net or mesh. In an example, this closure mechanism can be selected from the group consisting of: valve; electric detachment mechanism; elastic ring or band; threaded mechanism; sliding cover; sliding plug; filament loop; and electromagnetic solenoid. In an example, a closure mechanism can be a leaflet valve. In an example, a closure mechanism can be a one-way valve. In an example, a valve can allow embolic members and/or material to enter a flexible net or mesh through an opening in an annular member, but not allow the embolic members and/or material to exit the net or mesh.

In an example, a tubular mesh can be made from a polymer. In an example, a tubular mesh can be woven or braided from polymer threads, filaments, yarns, or strips. In an example, a tubular mesh can be 3D printed. In an example, a flexible net or mesh can be made from a flexible polymer. In an example, a flexible net or mesh can be made from an elastic and/or stretchable polymer. In an example, a flexible net or mesh can be elastic and/or stretchable and can expand as it is filled with embolic members and/or material. In an example, a flexible net or mesh can be sufficiently flexible to conform to the shape of even an irregularly-shaped aneurysm sac as the net or mesh is filled with embolic members and/or material. In an example, a flexible net or mesh can be sufficiently flexible to conform to the shape of even an irregularly-shaped (e.g. non-spherical) aneurysm sac as the net or mesh is filled with embolic members and/or material. In an example, a tubular mesh can be made from one or more materials selected from the group consisting of: Dacron, elastin, hydroxy-terminated polycarbonate, methylcellulose, nylon, PDMS, polybutester, polycaprolactone, polyester, polyethylene terephthalate, polypropylene, polytetrafluoroethene, polytetrafluoroethylene, polyurethane, silicone, and silk.

In an example, a tubular mesh can be made from metal. In an example, a tubular mesh can be made from Nitinol. In an example, a tubular mesh can be a flexible metal mesh. In an example, a tubular mesh can be a braided metal mesh. In an example, a tubular mesh can be woven or braided from metal filaments, wires, or tubes. In an example, a tubular mesh can be made from shape-memory material. In an example, a tubular mesh can be made with both metal and polymer components.

In an example, openings or holes in a flexible net or mesh can be smaller than the size (e.g. diameter, width, and/or length) of embolic members and/or material which is inserted into the net or mesh so that the embolic members and/or material do not escape out of the net or mesh. In an example, openings or holes in a flexible net or mesh can less than half of the size (e.g. diameter, width, and/or length) of embolic members and/or material which is inserted into the net or mesh so that the embolic members and/or material do not escape out of the net or mesh. In an example, openings or holes in a flexible net or mesh can have a size which is less than half of the smallest diameter and/or width of embolic members and/or material which is inserted into the net or mesh so that the embolic members and/or material do not escape out of the net or mesh. In an example, openings or holes in a flexible net or mesh can have a size which less than half of the smallest length of embolic members and/or material which is inserted into the net or mesh so that the embolic members and/or material do not escape out of the net or mesh.

In an example, a tubular mesh can have hexagonal openings In an example, a tubular mesh with hexagonal openings can be made using 3D printing. In an example, a flexible metal tubular mesh with hexagonal openings can be made by 3D printing with liquid metal. In an example, a tubular mesh with hexagonal openings can be made by 3D printing with a polymer. In an example, a tubular mesh with hexagonal openings can be made by 3D printing with an elastomeric polymer. In an example, a tubular mesh with hexagonal openings can be made by 3D printing with a silicone-based polymer. In an example, a tubular mesh with hexagonal openings can be made by 3D printing with polydimethylsiloxane (PDMS).

In an example, a tubular mesh can have quadrilateral openings. In an example, a tubular mesh with quadrilateral openings can be made using 3D printing. In an example, a flexible metal tubular mesh with quadrilateral openings can be made by 3D printing with liquid metal. In an example, a tubular mesh with quadrilateral openings can be made by 3D printing with a polymer. In an example, a tubular mesh with quadrilateral openings can be made by 3D printing with an elastomeric polymer. In an example, a tubular mesh with quadrilateral openings can be made by 3D printing with a silicone-based polymer. In an example, a tubular mesh with quadrilateral openings can be made by 3D printing with polydimethylsiloxane (PDMS).

In an example, a tubular mesh can have circular openings. In an example, a tubular mesh with circular openings can be made using 3D printing. In an example, a flexible metal tubular mesh with circular openings can be made by 3D printing with liquid metal. In an example, a tubular mesh with circular openings can be made by 3D printing with a polymer. In an example, a tubular mesh with circular openings can be made by 3D printing with an elastomeric polymer. In an example, a tubular mesh with circular openings can be made by 3D printing with a silicone-based polymer. In an example, a tubular mesh with circular openings can be made by 3D printing with polydimethylsiloxane (PDMS).

In an example, a tubular mesh can be made with a cobalt chromium alloy. In an example, a tubular mesh can be made with a nickel-titanium alloy. In an example, a tubular mesh can comprise cobalt chromium alloy wires, filaments, or tubes. In an example, a tubular mesh can comprise nickel-titanium alloy wires, filaments, or tubes. In an example, a tubular mesh can comprise nitinol wires, filaments, or tubes. In an example, a tubular mesh can be made with nitinol. In an example, a tubular mesh can comprise platinum wires, filaments, or tubes. In an example, a tubular mesh can be made with platinum. In an example, a tubular mesh can comprise stainless steel wires, filaments, or tubes. In an example, a tubular mesh can be made with stainless steel. In an example, a tubular mesh can comprise tantalum wires, filaments, or tubes. In an example, a tubular mesh can be made with tantalum.

In an example, a mid-section of tubular mesh can be more flexible than the proximal and distal sections of the tubular mesh to predispose the tubular mesh to fold over itself at that mid-section to more easily form a dual-layer flexible net or mesh via inversion or eversion of the tubular mesh. In an example, a mid-section of tubular mesh can be more flexible than the proximal and distal sections of the tubular mesh to predispose the tubular mesh to fold over itself at that mid-section to more easily form a dual-layer bowl-shaped flexible net or mesh via inversion or eversion of the tubular mesh. In an example, a mid-section of tubular mesh can be less dense than the proximal and distal sections of the tubular mesh to predispose the tubular mesh to fold over itself at that mid-section to more easily form a dual-layer flexible net or mesh via inversion or eversion of the tubular mesh.

In an example, a mid-section of tubular mesh can be less dense than the proximal and distal sections of the tubular mesh to predispose the tubular mesh to fold over itself at that mid-section to more easily form a dual-layer bowl-shaped flexible net or mesh via inversion or eversion of the tubular mesh. In an example, a mid-section of tubular mesh can be made with a lower-durometer material than the proximal and distal sections of the tubular mesh to predispose the tubular mesh to fold over itself at that mid-section to more easily form a dual-layer flexible net or mesh via inversion or eversion of the tubular mesh. In an example, a mid-section of tubular mesh can be made with a lower-durometer material than the proximal and distal sections of the tubular mesh to predispose the tubular mesh to fold over itself at that mid-section to more easily form a dual-layer bowl-shaped flexible net or mesh via inversion or eversion of the tubular mesh.

In an example, a distal portion (e.g. the distal half) of a flexible net or mesh can have a lower durometer than the proximal portion (e.g. the proximal half) of the flexible net or mesh. In an example, a distal portion (e.g. the distal half) of a flexible net or mesh can be more flexible than the proximal portion (e.g. the proximal half) of the flexible net or mesh. In an example, a distal portion (e.g. the distal half) of a flexible net or mesh can be less dense than the proximal portion (e.g. the proximal half) of the flexible net or mesh. In an example, a distal portion (e.g. the distal half) of a flexible net or mesh can be more porous than the proximal portion (e.g. the proximal half) of the flexible net or mesh.

In an example, a flexible net or mesh can be folded and/or compressed as it is delivered through a catheter to an aneurysm sac. In an example, a flexible net or mesh can have radial folds as it is delivered through a catheter to an aneurysm sac. In an example, a flexible net or mesh can have longitudinal folds as it is delivered through a catheter to an aneurysm sac. In an example, a flexible net or mesh can have cross-sectional folds as it is delivered through a catheter to an aneurysm sac.

In an example, a flexible net or mesh can have a longitudinal axis which spans in a proximal-to-distal direction. Proximal can be defined as being closer to the point of entry into a person's body during delivery through the person's vasculature (in the catheter) to the aneurysm and closer to the aneurysm neck after insertion into the aneurysm sac. In this example, a tubular mesh is transformed into a double-layer, distally-concave, bowl-shaped flexible net or mesh by two annular members (a proximal annular member and a distal annular member) which radially-constrain the proximal and distal ends of the tubular mesh, wherein the distal portion of the tubular mesh is inverted proximally (e.g. folded proximally) until it has a distally-concave shape. In this example, the distal circumference of the flexible net or mesh is a fold in the net or mesh. In this example, the proximal and distal annular members are aligned so that embolic members and/or material can be delivered through them into the distal-facing concavity of the double-layer bowl-shaped flexible net or mesh.

In an example, both of the radially-constrained ends can project into the interior of flexible net or mesh. In an example, the proximal end can be inverted to project into the interior of bowl-shaped flexible net or mesh and the distal end is not. Alternatively, a tubular mesh can be transformed into double-layer, distally-concave, bowl-shaped flexible net or mesh by a single annular member in a middle section (between the ends) of the tubular mesh which radially-constrains the middle of the tubular mesh, wherein the proximal portion of the tubular mesh is everted distally until it has a distally-concave shape. In an example, the distal circumference of the flexible net or mesh can comprise two nested tubular openings.

In an example, a tubular mesh is transformed into a single-layer, distally-concave, bowl-shaped flexible net or mesh by a single annular member which radially-constrains the proximal end of the tubular mesh. In an example, a tubular mesh can be transformed into single-layer, proximally-concave, bowl-shaped flexible net or mesh by a single annular member which radially-constrains the distal end of the tubular mesh.

In an example, a tubular mesh can be transformed into a single-layer ellipsoidal and/or generally globular flexible net or mesh by two annular members which radially-constrain the proximal and distal ends of the tubular mesh. In an example, both of these radially-constrained ends can be inverted to project into the interior of flexible net or mesh. In an example, the proximal end can be inverted to project into the interior of flexible net or mesh and the distal end can remain outside the interior of the flexible net or mesh. In an example, a tubular mesh is transformed into single-layer spherical flexible net or mesh by two annular members which radially-constrain the proximal and distal ends of the tubular mesh.

In an example, bound and/or inverted ends of a flexible net or mesh can both extend into the interior of a flexible net or mesh in a spherical, ellipsoidal, and/or globular configuration. In an example, a distal bound and/or inverted end of a flexible net or mesh can extend into the interior of a flexible net or mesh in a spherical, ellipsoidal, and/or globular configuration and a proximal bound and/or inverted end of the flexible net or mesh can extend outward from a flexible net or mesh in a spherical, ellipsoidal, and/or globular configuration. In an example, a proximal bound and/or inverted end of a flexible net or mesh can extend into the interior of a flexible net or mesh in a spherical, ellipsoidal, and/or globular configuration and a distal bound and/or inverted end of the flexible net or mesh can extend outward from a flexible net or mesh in a spherical, ellipsoidal, and/or globular configuration.

In an example, a tubular mesh can be made from polycarbonate urethane (PCU). In an example, a tubular mesh can be made from polydimethylsiloxane (PDMS). In an example, a tubular mesh can be made from polyesters. In an example, a tubular mesh can be made from polyether block amide (PEBA). In an example, a tubular mesh can be made from polyetherether ketone (PEEK). In an example, a tubular mesh can be made from polyethylene. In an example, a tubular mesh can be made from polyethylene glycol (PEG). In an example, a tubular mesh can be made from polyethylene terephthalate (PET).

In an example, a tubular mesh can be made from polyglycolic acid (PGA). In an example, a tubular mesh can be made from polylactic acid (PLA). In an example, a tubular mesh can be made from poly-N-acetylglucosamine (PNAG). In an example, a tubular mesh can be made from polyolefin. In an example, a tubular mesh can be made from polyoleandlena. In an example, a tubular mesh can be made from polypropylene. In an example, a tubular mesh can be made from polytetrafluoroethylene (PTFE). In an example, a tubular mesh can be made from polyurethane (PU). In an example, a tubular mesh can be made from polywanacrakor. In an example, a tubular mesh can be made from polyvinyl alcohol (PVA). In an example, a tubular mesh can be made from polyvinyl pyrrolidone (PVP).

In an example, a tubular mesh from which a flexible net or mesh is formed can be tapered. In an example, the distal end of a tubular mesh can have a smaller diameter than the proximal end of the tubular mesh. In an example, the distal end of a tubular mesh can have a larger diameter than the proximal end of the tubular mesh. In an example, a tubular mesh from which a flexible net or mesh is formed can have differential flexibility. In an example the distal portion of a tubular mesh can have a first level of flexibility and the proximal portion of the tubular mesh can have a second level of flexibility, wherein the first level is less than the second level. In an example the distal portion of a tubular mesh can have a first level of flexibility and the proximal portion of the tubular mesh can have a second level of flexibility, wherein the first level is greater than the second level.

In an example, a tubular mesh from which a flexible net or mesh is formed can have differential porosity. In an example the distal portion of a tubular mesh can have a first porosity level and the proximal portion of the tubular mesh can have a second porosity level, wherein the first level is less than the second level. In an example the distal portion of a tubular mesh can have a first porosity level and the proximal portion of the tubular mesh can have a second porosity level, wherein the first level is greater than the second level. In an example, a tubular mesh from which a flexible net or mesh is formed can have differential durometer. In an example the distal portion of a tubular mesh can have a first durometer level and the proximal portion of the tubular mesh can have a second durometer level, wherein the first level is less than the second level. In an example the distal portion of a tubular mesh can have a first durometer level and the proximal portion of the tubular mesh can have a second durometer level, wherein the first level is greater than the second level.

In an example, the width of a flexible net or mesh in a bowl-shaped configuration can be larger than the width of the aneurysm neck. In an example, the circumference of a flexible net or mesh in a bowl-shaped configuration can be larger than the circumference of the aneurysm neck. In an example, the width of a flexible net or mesh in a bowl-shaped configuration can be at least 10% larger than the width of the aneurysm neck. In an example, the circumference of a flexible net or mesh in a bowl-shaped configuration can be at least 10% larger than the circumference of the aneurysm neck. In an example, the width of a flexible net or mesh in a bowl-shaped configuration can be at least 90% of the maximum width of the aneurysm sac (parallel to the aneurysm neck). In an example, the circumference of a flexible net or mesh in a bowl-shaped configuration can be at least 90% of the circumference of the maximum circumference of the aneurysm sac (parallel to the aneurysm neck). In an example, a flexible net or mesh can function as a neck bridge, reducing or completely blocking blood flow from the parent vessel into the aneurysm sac.

In an example, a flexible net or mesh formed from a tubular mesh can have a generally-hemispherical shape after a tubular mesh has been radially-constrained by one or more annular members. In an example, a flexible net or mesh formed from a tubular mesh can have a generally globular and/or spherical shape after a tubular mesh has been radially-constrained by one or more annular members. In an example, a flexible net or mesh formed from a tubular mesh can have an ellipsoidal or oval shape after a tubular mesh has been radially-constrained by one or more annular members. In an example, a flexible net or mesh formed from a tubular mesh can have a disk shape after a tubular mesh has been radially-constrained by one or more annular members.

In an example, a flexible net or mesh formed from a tubular mesh can have the shape of a paraboloid-of-revolution (e.g. a paraboloid revolved around a left or right vertical edge) after a tubular mesh has been radially-constrained by one or more annular members. In an example, a flexible net or mesh formed from a tubular mesh can comprise a carlavian curve shape after a tubular mesh has been radially-constrained by one or more annular members. In an example, a flexible net or mesh formed from a tubular mesh can have a toroidal shape after a tubular mesh has been radially-constrained by one or more annular members. In an example, a flexible net or mesh formed from a tubular mesh can have a half-toroidal shape (e.g. a sliced bagel shape) after a tubular mesh has been radially-constrained by one or more annular members.

In an example, the distal end of a tubular mesh can be radially-constrained by a distal annular member and the proximal end of a tubular mesh can be radially-constrained by a proximal annular member to form a generally-globular, spherical, and/or ellipsoidal flexible net or mesh which is inserted into an aneurysm sac. In an example, the distal end of a tubular mesh can be radially-constrained by a distal annular member and the proximal end of a tubular mesh can be radially-constrained by a proximal annular member to form a generally-globular, spherical, and/or ellipsoidal shape, wherein the distal portion is then inverted and/or folded to create a two-layer bowl-shaped flexible net or mesh which is inserted into an aneurysm sac. In an example, both the distal end of a tubular mesh and the proximal end of a tubular mesh can be radially-constrained by a proximal annular member to form a two-layer bowl-shaped flexible net or mesh which is inserted into an aneurysm sac.

In an example a flexible net or mesh can be a two-layer bowl-shaped mesh with a distally-concave proximal layer and a distally-concave distal layer. In an example a flexible net or mesh can be a two-layer bowl-shaped mesh with a distally-concave proximal layer and a distally-concave distal layer, wherein the distance between the proximal and distal layers is greater in a radially-central portion of the flexible net or mesh than in radially-peripheral portions of the flexible net or mesh. In an example a flexible net or mesh can be a two-layer bowl-shaped mesh with a proximal layer and a distal layer, wherein the proximal layer has a uniform distal-facing concavity, but the distal layer has locally-concave and locally-convex portions. In an example, the radially-central portion of the distal layer is locally-convex and the radially-peripheral portions of the distal layer are locally-concave. In an example, the radially-central portion of the distal layer is less distally-concave than the radially-peripheral portions of the distal layer.

In an example, embolic members and/or material which is inserted into the flexible net or mesh can be microspheres or microballs. In an example, embolic members and/or material inserted into the flexible net or mesh can be microsponges. In an example, embolic members and/or material inserted into the flexible net or mesh can be pieces of foam. In an example, embolic members and/or material inserted into the flexible net or mesh can be microbeads. In an example, embolic members and/or material inserted into the flexible net or mesh can be pieces of hydrogel. In an example, embolic members and/or material inserted into the flexible net or mesh can be metal embolic coils. In an example, embolic members and/or material inserted into the flexible net or mesh can be embolic ribbons. In an example, embolic members and/or material inserted into the flexible net or mesh can be yarns or filaments. In an example, embolic members and/or material can be polymer strands or coils. In an example, accumulation of embolic members and/or material in an aneurysm sac can compress a flexible net or mesh from a spherical, ellipsoidal, and/or globular configuration into a hemispherical, bowl-shaped, and/or distally-concave configuration by pressing against the distal surface of the flexible net or mesh.

In an example, embolic members and/or material inserted into the flexible net or mesh can be microspheres or microballs connected by a longitudinal wire, cord, and/or filament (e.g. in a “string-of-pearls” configuration). In an example, embolic members and/or material inserted into the flexible net or mesh can be microsponges connected by a longitudinal wire, cord, and/or filament (e.g. in a “string-of-pearls” configuration). In an example, embolic members and/or material inserted into the flexible net or mesh can be pieces of foam connected by a longitudinal wire, cord, and/or filament (e.g. in a “string-of-pearls” configuration). In an example, embolic members and/or material inserted into the flexible net or mesh can be microbeads connected by a longitudinal wire, cord, and/or filament (e.g. in a “string-of-pearls” configuration).

In an example, embolic members and/or material inserted into the flexible net or mesh can be pieces of hydrogel connected by a longitudinal wire, cord, and/or filament (e.g. in a “string-of-pearls” configuration). In an example, embolic members and/or material inserted into the flexible net or mesh can be embolic coils connected by a longitudinal wire, cord, and/or filament (e.g. in a “string-of-pearls” configuration). In an example, embolic members and/or material inserted into the flexible net or mesh can be embolic ribbons connected by a longitudinal wire, cord, and/or filament (e.g. in a “string-of-pearls” configuration). In an example, embolic members and/or material inserted into the flexible net or mesh can be yarns or filaments connected by a longitudinal wire, cord, and/or filament (e.g. in a “string-of-pearls” configuration).

In an example, embolic members and/or material inserted into the flexible net or mesh can be liquid which congeals and/or solidifies. In an example, embolic members and/or material inserted into the flexible net or mesh can be a polymer which congeals and/or solidifies. In an example, embolic members and/or material inserted into the flexible net or mesh can be a liquid embolic material. In an example, embolic members and/or material inserted into the flexible net or mesh can be hydrogel material. In an example, embolic members and/or material inserted into the flexible net or mesh can be congealing adhesive material. In an example, accumulation of embolic members and/or material in an aneurysm sac can compress a flexible net or mesh from a spherical, ellipsoidal, and/or globular configuration to a hemispherical, bowl-shaped, and/or distally-concave configuration by pressing against the distal surface of the flexible net or mesh.

In an example, embolic members and/or material which is inserted through an annular member into a flexible net or mesh can be one or more mesh ribbons. In an example, embolic members and/or material which is inserted through an annular member into a flexible net or mesh can be one or more wire mesh ribbons. In an example, embolic members and/or material which is inserted through an annular member into a flexible net or mesh can be one or more polymer mesh ribbons. In an example, embolic members and/or material which is inserted through an annular member into a flexible net or mesh can be one or more undulating and/or sinusoidal ribbons. In an example, embolic members and/or material which is inserted through an annular member into a flexible net or mesh can be one or more double-layer mesh ribbons.

In an example, embolic members and/or material can be made with a cobalt chromium alloy. In an example, embolic members and/or material can be made with a nickel-titanium alloy. In an example, embolic members and/or material can be cobalt chromium alloy coils or ribbons. In an example, embolic members and/or material can be nickel-titanium alloy coils or ribbons. In an example, embolic members and/or material can be nitinol coils or ribbons. In an example, embolic members and/or material can be made with nitinol. In an example, embolic members and/or material can be platinum coils or ribbons. In an example, embolic members and/or material can be made with platinum. In an example, embolic members and/or material can be stainless steel coils or ribbons. In an example, embolic members and/or material can be made with stainless steel. In an example, embolic members and/or material can be tantalum coils or ribbons. In an example, embolic members and/or material can be made with tantalum.

In an example, embolic members and/or material can be pushed through a catheter into a flexible net or mesh by a pusher wire and/or plug. In an example, liquid embolic material (which congeals after insertion into the net or mesh) can be pushed through a catheter into a flexible net or mesh by fluid pressure. In an example, embolic members can be pushed into a flexible net or mesh by a flow of liquid (e.g. saline solution), wherein the embolic members are retained in the flexible net or mesh and the saline solution escapes out of openings in the flexible net or mesh. In an example, embolic members and/or material can be pushed through a catheter into a flexible net or mesh by a conveyer belt mechanism. In an example, embolic members and/or material can be pushed through a catheter into a flexible net or mesh by a rotating helical delivery mechanism.

In an example, embolic members which are inserted into a net or mesh can be embolic coils or ribbons. In an example, embolic members which are inserted into a net or mesh can be pieces of foam or gel (such as hydrogel). In an example, embolic members which are inserted into a net or mesh can be microballs or microspheres. In an example, embolic members which are inserted into a net or mesh can be microsponges. In an example, embolic members which are inserted into a net or mesh can be filaments or yarns. In an example, liquid embolic material can be inserted into a net or mesh.

In an example, embolic members which are inserted into a net or mesh can be selected from the group consisting of: pieces of gel; pieces of foam; and micro-sponges. In an example, embolic members which are inserted into a net or mesh can be pieces of gel, such as hydrogel. In an example, embolic members which are inserted into a net or mesh can be pieces of foam. In an example, embolic members which are inserted into a net or mesh can be micro-sponges. In an example, embolic members which are inserted into a net or mesh can be microscale gel balls. In an example, embolic members which are inserted into a net or mesh can be microscale foam balls. In an example, embolic members which are inserted into a net or mesh can be microscale sponge balls. In an example, embolic members which are inserted into a net or mesh can be microscale gel polyhedrons. In an example, embolic members which are inserted into a net or mesh can be microscale foam polyhedrons. In an example, embolic members which are inserted into a net or mesh can be microscale sponge polyhedrons.

In an example, embolic members which are inserted into a net or mesh can have generally spherical or globular shapes. In an example, embolic members which are inserted into a net or mesh can have generally prolate spherical, ellipsoidal, or ovaloid shapes. In an example, embolic members which are inserted into a net or mesh can have apple, barrel, or pair shapes. In an example, embolic members which are inserted into a net or mesh can have torus or ring shapes. In an example, embolic members which are inserted into a net or mesh can have disk or pancake shapes. In an example, embolic members which are inserted into a net or mesh can have peanut or hour-glass shapes. In an example, embolic members which are inserted into a net or mesh can be polyhedrons comprised of hexagonal surfaces. In an example, embolic members which are inserted into a net or mesh can be polyhedrons comprised of quadrilateral surfaces. In an example, embolic members which are inserted into a net or mesh can be polyhedrons comprised of triangular surfaces.

In an example, an embolic member can have a shape which is selected from the group consisting of: apple-shaped, barrel-shaped, bulbous, convex, ellipsoidal, globular, oblate spheroid, ovaloid, prolate-spheroid-shaped, spherical, and truncated-sphere-shaped. In an example, an embolic member can have a shape which is selected from the group consisting of: bowl-shaped, concave, hemispherical, and paraboloid of revolution. In an example, an embolic member can have a shape which is selected from the group consisting of: cubic, hexagon-shaped, hexahedron, octagon-shaped, octahedron, pentagonal-shaped, polyhedron-shaped, pyramidal, rectangular, square, and tetrahedronal.

In an example, embolic members which are inserted into a net or mesh can have a (diameter) size within the range of 0.5 to 2 millimeters. In an example, embolic members which are inserted into a net or mesh can have a (diameter) size within the range of 1 to 5 millimeters. In an example, embolic members which are inserted into a net or mesh can have a (diameter) size within the range of 2 to 10 millimeters. In an example, embolic members which are inserted into a net or mesh can have a (diameter) size within the range of 5 to 20 millimeters. In an example, embolic members which are inserted into a net or mesh can have a (diameter) size within the range of 0.5 to 2 microns. In an example, embolic members which are inserted into a net or mesh can have a (diameter) size within the range of 1 to 5 microns. In an example, embolic members which are inserted into a net or mesh can have a (diameter) size within the range of 2 to 10 microns. In an example, embolic members which are inserted into a net or mesh can have a (diameter) size within the range of 5 to 20 microns.

In an example, between 5 and 20 embolic members can be inserted into a net or mesh. In an example, between 10 and 50 embolic members can be inserted into a net or mesh. In an example, between 20 and 100 embolic members can be inserted into a net or mesh. In an example, between 50 and 500 embolic members can be inserted into a net or mesh.

In an example, embolic members which are inserted into a net or mesh can expand in size within the net or mesh. In an example, embolic members can have a first (average) size while being delivered to an aneurysm sac via a micro-catheter and a second (average) size after expansion within the aneurysm sac, wherein the second (average) size is 10% to 50% larger than the first (average) size. In an example, embolic members can have a first (average) size while being delivered to an aneurysm sac via a micro-catheter and a second (average) size after expansion within the aneurysm sac, wherein the second (average) size is 40% to 100% larger than the first (average) size. In an example, embolic members can have a first (average) size while being delivered to an aneurysm sac via a micro-catheter and a second (average) size after expansion within the aneurysm sac, wherein the second (average) size is more than twice the first (average) size.

In an example, embolic members can self-expand within a net or mesh after they are released from a delivery catheter. In an example, embolic members can swell upon hydration from interaction with blood or other body fluid. In an example, embolic members can be expanded within the net or mesh by one or more mechanisms selected from the group consisting of: expansion due to interaction with body fluid; expansion due to application of thermal energy; expansion due to exposure to a chemical agent; and expansion due to exposure to light energy. In an example, embolics can expand by a factor of 2-5 times. In an example, embolics can expand by a factor of 4-10 times. In an example, embolics can expand by a factor of more than 10 times. In an example, embolic members can expand to a sufficiently-large size that they cannot escape from the net or mesh after insertion into the net or mesh.

In an example, three-dimensional embolic members which are inserted into a net or mesh can be soft and compressible. In an example, three-dimensional embolic members which are inserted into a net or mesh can have a durometer less than 50. In an example, three-dimensional embolic members which are inserted into a net or mesh can have an average durometer within the range of 10 to 30. In an example, three-dimensional embolic members which are inserted into a net or mesh can have an average durometer within the range of 25 to 50. In an example, three-dimensional embolic members which are inserted into a net or mesh can have an average durometer which is less than 70.

In an example, embolic members which are inserted into a net or mesh can be made from a polymer. In an example, embolic members which are inserted into a net or mesh can be made from an elastomeric polymer. In an example, embolic members which are inserted into a net or mesh can be made from a silicone-based polymer. In an example, embolic members which are inserted into a net or mesh can be made from polydimethylsiloxane (PDMS).

In an example, an embolic member can further comprise one or more layers made with different materials. In an example, an inner layer of an embolic member can be made from a first material and an outer layer of an embolic member can be made from a second material. In an example, an inner layer of an embolic member can be made from a first material with a first durometer and an outer layer of an embolic member can be made from a second material with a second durometer, wherein the second durometer is less than the first durometer. In an example, an embolic member can have an outer layer which is adhesive. In an example, an embolic member can have an outer layer with an adhesive property which is activated by application of electromagnetic and/or thermal energy. In an example, an embolic member can have an outer layer with an adhesive property which is activated by interaction with blood.

In an example, there can be a first average durometer of embolic members which are inserted into the net or mesh at a first time and a second average durometer of embolic members which are inserted into the net or mesh at a second time, wherein the second average durometer is greater than the first average durometer. In an example, there can be a first average durometer of embolic members which are inserted into the net or mesh at a first time and a second average durometer of embolic members which are inserted into the net or mesh at a second time, wherein the second average durometer is less than the first average durometer.

In an example, there can be a first average length of longitudinal strands between proximal pairs of embolic members which are inserted into a net or mesh at a first time, a second average length of longitudinal strands between proximal pairs of embolic members which are inserted into the net or mesh at a second time, and the second average length can be greater than the first average length. In an example, there can be a first average length of longitudinal strands between proximal pairs of embolic members which are inserted into a net or mesh at a first time, a second average length of longitudinal strands between proximal pairs of embolic members which are inserted into the net or mesh at a second time, and the second average length can be less than the first average length.

In an example, there can be a first set of embolic members which are inserted into a net or mesh at a first time and a second set of embolic members which are inserted into the net or mesh at a second time, wherein the second set of embolic members are closer together than the first set of embolic members. In an example, there can be a first set of embolic members which are inserted into a net or mesh at a first time and a second set of embolic members which are inserted into the net or mesh at a second time, wherein the first set of embolic members are closer together than the second set of embolic members. In an example, there can be a longitudinal series of embolic members connected by one or more longitudinal strands which is inserted into a net or mesh within an aneurysm sac, wherein embolic members in the longitudinal series are progressively closer to each other moving along the length of the series in a distal to proximal manner. In an example, there can be a longitudinal series of embolic members connected by one or more longitudinal strands which is inserted into a net or mesh within an aneurysm sac, wherein embolic members in the longitudinal series are progressively farther from each other moving along the length of the series in a distal to proximal manner.

In an example, embolic members which are inserted into the net or mesh at a first time can have first shapes, embolic members which are inserted into the net or mesh at a second time can have second shapes, and the second shape can be different than the first shape. In an example, embolic members which are inserted into the net or mesh at a first time can be made with a first (combination of) material, embolic members which are inserted into the net or mesh at a second time can be made with a second (combination of) material, and the second (combination of) material can be different from the first (combination of) material. In an example, embolic members which are inserted into the net or mesh at a first time can be made with a first (combination of) material, embolic members which are inserted into the net or mesh at a second time can be made with a second (combination of) material, and the second (combination of) material can be more flexible, elastic, and/or compliant than the first (combination of) material.

In an example, embolic members which are inserted into the net or mesh at a first time can be made with a first (combination of) material, embolic members which are inserted into the net or mesh at a second time can be made with a second (combination of) material, and the second (combination of) material can have a lower durometer than the first (combination of) material. In an example, embolic members which are inserted into the net or mesh at a first time can be made with a first (combination of) material, embolic members which are inserted into the net or mesh at a second time can be made with a second (combination of) material, and the second (combination of) material can be less flexible, elastic, and/or compliant than the first (combination of) material. In an example, embolic members which are inserted into the net or mesh at a first time can be made with a first (combination of) material, embolic members which are inserted into the net or mesh at a second time can be made with a second (combination of) material, and the second (combination of) material can have a higher durometer than the first (combination of) material.

In an example, there can be a first average size of embolic members which are inserted into the net or mesh at a first time, a second average size of embolic members which are inserted into the net or mesh at a second time, and the second average size can be greater than the first average size. In an example, there can be a first average size of embolic members which are inserted into the net or mesh at a first time, a second average size of embolic members which are inserted into the net or mesh at a second time, and the second average size can be less than the first average size.

In an example, a net or mesh can be delivered into an aneurysm sac via a catheter and/or delivery tube. In an example, a plurality of embolic members can be delivered into the net or mesh via the same catheter and/or delivery tube. In an example, a net or mesh can be delivered into an aneurysm sac via a first catheter and/or delivery tube and a plurality of embolic members can be delivered into the net or mesh via a second catheter and/or delivery tube.

In an example, embolic members can be made from ethylene vinyl alcohol (EVA). In an example, embolic members can be made from polyolefin. In an example, embolic members can be made from fibrinogen. In an example, embolic members can be made from polylactic acid (PLA). In an example, embolic members can be made from polyethylene terephthalate (PET). In an example, embolic members can be made from steel (e.g. stainless steel). In an example, embolic members can be made from methylcellulose.

In an example, embolic members can be made from acrylic. In an example, embolic members can be made from polyethylene glycol (PEG). In an example, embolic members can be made from silk. In an example, embolic members can be made from alginate. In an example, embolic members can be made from gold. In an example, embolic members can be made from polyethylene. In an example, embolic members can be made from polyoleandlena. In an example, embolic members can be made from tantalum. In an example, embolic members can be made from cobalt-chrome alloy (cobalt chromium).

In an example, embolic members can be made from polyetherether ketone (PEEK). In an example, embolic members can be made from polywanacrakor. In an example, embolic members can be made from thermoplastic elastomer. In an example, embolic members can be made from polycarbonate urethane (PCU). In an example, embolic members can be made from water-soluble synthetic polymer. In an example, embolic members can be made from collagen. In an example, embolic members can be made from polyvinyl alcohol (PVA).

In an example, embolic members can be made from titanium. In an example, embolic members can be made from polyether block amide (PEBA). In an example, embolic members can be made from radiopaque material. In an example, embolic members can be made from copolymer. In an example, embolic members can be made from polyvinyl pyrrolidone (PVP). In an example, embolic members can be made from polydimethylsiloxane (PDMS). In an example, embolic members can be made from zirconium-based alloy. In an example, embolic members can be made from polyesters. In an example, embolic members can be made from hydrogel. In an example, embolic members can be made from silicone. In an example, embolic members can be made from nitinol (or other nickel titanium alloy).

In an example, embolic members can be made from polyglycolic acid (PGA). In an example, embolic members can be made from small intestinal submucosa. In an example, embolic members can be made from nylon. In an example, embolic members can be made from polypropylene. In an example, embolic members can be made from platinum. In an example, embolic members can be made from polyurethane (PU). In an example, embolic members can be made from tungsten. In an example, embolic members can be made from fibrin.

In an example, embolic members can be made from poly-N-acetylglucosamine (PNAG). In an example, embolic members can be made from latex. In an example, embolic members can be made from fibronectin. In an example, embolic members can be made from palladium. In an example, embolic members can be made from polytetrafluoroethylene (PTFE). In an example, embolic members can be made from gelatin.

In an example, a selected quantity, series, length, and/or volume of embolic members can be selectively dispensed and/or detached into the net or mesh in situ by a mechanism selected from the group consisting of: breaking a connection between embolic members in a series of embolic members; cutting a connection between embolic members in a series of embolic members (e.g. with a cutting edge or laser); dissolving a connection between embolic members in a series of embolic members (e.g. with thermal energy or a chemical); electrolytic mechanism; hydraulic mechanism; injecting a flow of embolic members suspended in a liquid or gel into a net or mesh; melting a connection between embolic members in a series of embolic members (e.g. with thermal or light energy); progressing embolic members into a net or mesh via a conveyor belt (e.g. chain-based conveyor); progressing embolic members into a net or mesh via a helical conveyor (e.g. with an Archimedes' screw); pushing embolic members into a net or mesh using the force of a liquid flow; pusher rod and/or plunger; release detachment mechanism; and thermal detachment mechanism.

In an example, embolic members can differ among themselves with respect to one or more characteristics selected from the group consisting of: porosity, shape, size, material, composition, coating, radiopacity, strength, stiffness, and type. In an example, a plurality of embolic members can be delivered into a net or mesh in a linear (longitudinal) array or series of inter-connected embolic members. In an example, a plurality of embolic members can be delivered into a net or mesh in a linear (longitudinal) array of connected embolic members, wherein this linear array can be cut, separated, and/or detached in situ (in a remote manner) at one or more selected locations by the user of the device in order to deliver a selected quantity, length, or volume or embolic members. In an example, a plurality of embolic members can be delivered into a net or mesh in a planar array of inter-connected embolic members. In an example, a plurality of embolic members can be delivered into a net or mesh in a three-dimensional array of inter-connected embolic members.

In an example, a series of embolic members can be delivered into a net or mesh, wherein successive embolic members in the series are closer together. In an example, a series of embolic members can be delivered into a net or mesh, wherein embolic members in the series are progressively closer together (as one progresses along the series in a distal to proximal manner). In an example, a series of embolic members can be delivered into a net or mesh, wherein successive embolic members in the series are farther apart from each other. In an example, a series of embolic members can be delivered into a net or mesh, wherein embolic members in the series are progressively farther apart (as one progresses along the series in a distal to proximal manner).

In an example, a series of embolic members can be delivered into a net or mesh, wherein successive embolic members in the series decrease in durometer. In an example, a series of embolic members can be delivered into a net or mesh, wherein embolic members in the series have progressively lower durometer values (as one progresses along the series in a distal to proximal manner). In an example, a series of embolic members can be delivered into a net or mesh, wherein successive embolic members in the series increase in durometer. In an example, a series of embolic members can be delivered into a net or mesh, wherein embolic members in the series have progressively higher durometer values (as one progresses along the series in a distal to proximal manner).

In an example, a series of embolic members can be delivered into a net or mesh, wherein successive embolic members in the series are made of different materials. In an example, a series of embolic members can be delivered into a net or mesh, wherein successive embolic members in the series are made of different materials, wherein these materials differ in porosity. In an example, a series of embolic members can be delivered into a net or mesh, wherein successive embolic members in the series are made of different materials, wherein these materials differ in radiopacity. In an example, a series of embolic members can be delivered into a net or mesh, wherein successive embolic members in the series are made of different materials, wherein these materials differ in stiffness. In an example, a series of embolic members can be delivered into a net or mesh, wherein successive embolic members in the series are made of different materials, wherein these materials differ in durometer.

In an example, a series of embolic members can be delivered into a net or mesh, wherein successive embolic members in the series decrease in porosity. In an example, a series of embolic members can be delivered into a net or mesh, wherein embolic members in the series become progressively less porous (as one progresses along the series in a distal to proximal manner). In an example, a series of embolic members can be delivered into a net or mesh, wherein successive embolic members in the series increase in porosity. In an example, a series of embolic members can be delivered into a net or mesh, wherein embolic members in the series become progressively more porous (as one progresses along the series in a distal to proximal manner).

In an example, a series of embolic members can be delivered into a net or mesh, wherein successive embolic members in the series differ in shape. In an example, a series of embolic members can be delivered into a net or mesh, wherein successive embolic members in the series differ in their degree of convexity. In an example, a series of embolic members can be delivered into a net or mesh, wherein successive embolic members in the series differ in their degree of concavity.

In an example, a series of embolic members can be delivered into a net or mesh, wherein successive embolic members in the series decrease in size. In an example, a series of embolic members can be delivered into a net or mesh, wherein embolic members in the series become progressively smaller (as one progresses along the series in a distal to proximal manner). In an example, a series of embolic members can be delivered into a net or mesh, wherein successive embolic members in the series increase in size. In an example, a series of embolic members can be delivered into a net or mesh, wherein embolic members in the series become progressively larger (as one progresses along the series in a distal to proximal manner).

In an example, embolic members can be soft, compressible members such as microsponges or blobs of gel. In an example, embolic members can be made from sponge, foam, or gel. In an example, embolic members can be hard, uncompressible members such as hard polymer spheres or beads. In an example, embolic members can be made from one or more materials selected from the group consisting of: cellulose, collagen, acetate, alginic acid, carboxy methyl cellulose, chitin, collagen glycosaminoglycan, divinylbenzene, ethylene glycol, ethylene glycol dimethylmathacrylate, ethylene vinyl acetate, hyaluronic acid, hydrocarbon polymer, hydroxyethylmethacrylate, methlymethacrylate, polyacrylic acid, polyamides, polyesters, polyolefins, polysaccharides, polyurethane, polyvinyl alcohol, silicone, urethane, and vinyl stearate.

In an example, embolic members can have a shape selected from the group consisting of: ball or sphere, ovoid, ellipsoid, and polyhedron. In an example, embolic members can have a Shore OO value, indicative of softness or hardness, within a range of 5 to about 50. In an example, embolic members can have a diameter or like size within a range of 50 micrometers to 2000 micrometers. In an example, differently-sized embolic members can be used. In an example two or more different sizes of embolic members can be inserted into a net or mesh to occlude an aneurysm. In an example, embolic members can include small balls and large balls. In an example, it may be advantageous to first fill a net or mesh with larger balls and then continue filling the net or mesh with smaller balls. In another example, it may be advantageous to first fill a net or mesh with smaller balls and then continue filling the net or mesh with larger balls.

In an example, an intrasaccular aneurysm occlusion device can be filled with a “string of pearls” string (or wire) connected sequence of embolic members. In an example, an intrasaccular aneurysm occlusion device can include a series of embolic members which are connected by a strand. In an example, a device can include a string of pearls” series of embolic members which are linked by a strand (e.g. a thin flexible member). In an example, a device can include a string of pearls” series of embolic members which are centrally linked by a strand (e.g. a thin flexible member). In an example, a “string of pearls” string-or-wire connected sequence of embolic members can comprise a plurality of embolic members which are separate from each other, but pair-wise connected to each other by at least one string or wire. In an example, a plurality of members can be unevenly-spaced along the longitudinal axis of a flexible member. In an example, uneven spacing of the embolic members can be selected based on the size and shape of an aneurysm to be occluded. In an example, the distances between embolic members can vary. In an example, the space between embolic members can differ for occlusion of narrow-neck aneurysms vs. wide-neck aneurysms. In an example, distances between embolic members can become progressively shorter in a distal to proximal direction.

In an example, a line which connects embolic members can be a wire, spring, or chain. In an example, a connecting line can be a string, thread, band, fiber, or suture. In an example, embolic members can be centrally connected to each other by a connecting line. In an example, the centroids of embolic members can be connected by a connecting line. In an example, expanding arcuate embolic members can slide (e.g. up or down) along a connecting line. In an example, embolic members can slide along a connecting line, but only in one direction. In an example, a connecting line can have a ratchet structure which allows embolic members to slide closer to each other but not slide further apart. In an example, this device can further comprise a locking mechanism which stops embolic members from sliding along a connecting line. In an example, application of electromagnetic energy to a connecting line can fuse the line with the embolic members and stop them from sliding, effectively locking them in proximity to each other.

In an example, embolic members can be conveyed through a lumen to an aneurysm in a fluid flow, wherein the fluid escapes out from a net or mesh and the embolic members are retained within the net or mesh. In an example, embolic members can be conveyed through a lumen to an aneurysm by means of a moving belt or wire loop. In an example, embolic members can be conveyed through a lumen to an aneurysm by means of an Archimedes screw.

In an example, a flexible net or mesh can self-expand to a first extent after being released from a catheter into an aneurysm sac. In an example, the flexible net or mesh can further expand, to a second extent, due to pressure from the accumulation of embolic members and/or embolic material within its interior and/or distal-facing concavity. Other example variations discussed elsewhere in this disclosure or in priority-linked disclosures can also be applied to this example where relevant.

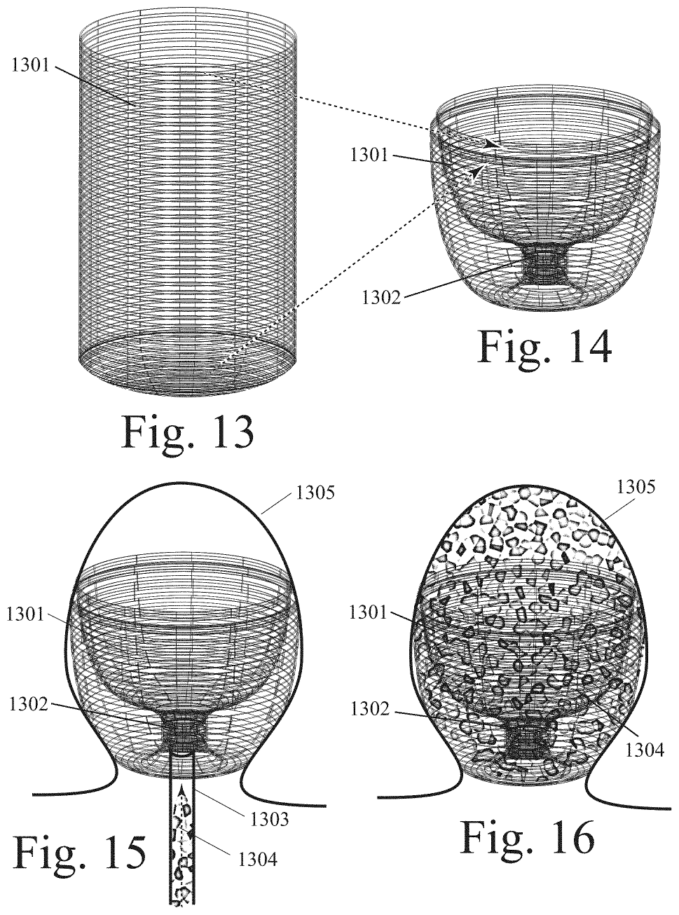

FIGS. 13 through 16 show four views, at different times, of the formation and deployment of another example of an intrasacular aneurysm occlusion device comprising: at least one annular member (in this example, a single mid-section annular member 1302), wherein an annular member is selected from the group consisting of a ring, a band, a cylinder, a tube, and a catheter; a flexible net or mesh, wherein the flexible net or mesh has a spherical, ellipsoidal, generally-globular, hemispherical, and/or bowl-shaped first configuration when it is formed by encircling, pinching, inverting, and/or everting a tubular mesh 1301 at one or more longitudinal locations using the at least one annular member; wherein the flexible net or mesh has a radially-compressed second configuration for delivery through a catheter 1303 into an aneurysm sac 1305; and wherein the flexible net or mesh is inserted and expanded within the aneurysm sac; and embolic members and/or embolic material 1304 which is inserted into the interior and/or the distal-facing concavity of the flexible net or mesh through one or more of the annular members. In this example, there is a single annular member which radially constrains a mid-section of the tubular mesh. In this example, the flexible net or mesh has a double-layer bowl shape when it is first formed from the tubular mesh.

FIG. 13 introduces tubular mesh 1301 which is used to make the intrasacular aneurysm occlusion device. FIG. 14 shows a single mid-section annular member 1302 which radially-constrains a mid-section of the tubular mesh. This mid-section radial constraint first transforms the tubular mesh into an hourglass-shaped mesh, which is then transformed into a two-layer bowl-shaped flexible mesh by everting the proximal portion of the mesh over the distal portion of the mesh as shown in FIG. 14. FIG. 15 shows this flexible mesh after it has been radially-compressed for delivery through a catheter 1303 into an aneurysm sac 1305, wherein the flexible mesh has been inserted into the aneurysm sac, and wherein embolic members and/or material 1304 are starting to be delivered through the catheter (and through the annular member) into the distal-facing concavity of the bowl-shaped flexible mesh and the distal portion of the aneurysm sac. FIG. 16 shows the distal-facing concavity of the bowl-shaped flexible mesh and the distal portion of aneurysm sac having been filed with embolic members and/or material and the catheter having been removed.

In this example, an annular member is a ring or band which encircles a middle portion (between the ends) of the tubular mesh. In an example, an annular member can be a metal ring, band, or cylinder. In an example, an annular member can be a polymer ring, band, or cylinder. In an example, an annular member can be a wire, cord, or string. In an example, an annular member can be a ring or band which encircles a tubular mesh, thereby radially-constraining and/or pinching the tubular mesh but allowing embolic members and/or embolic material to pass through it into the interior and/or a concavity of the flexible net or mesh. In an example, an annular member can be a cylinder which encircles a tubular mesh, thereby radially-constraining and/or pinching the tubular mesh but allowing embolic members and/or embolic material to pass through it into the interior and/or a concavity of the flexible net or mesh.

In an example, an annular member can be a cord or wire which encircles a tubular mesh, thereby radially-constraining and/or pinching the tubular mesh but allowing embolic members and/or embolic material to pass through it into the interior and/or a concavity of the flexible net or mesh. In an example, an annular member can be a catheter or tube around which a tubular mesh is attached, thereby radially-constraining and/or pinching the tubular mesh but allowing embolic members and/or embolic material to pass through it into the interior and/or a concavity of the flexible net or mesh. In an example, an annular member can be a lumen through a flexible net or mesh through which embolic members and/or material is inserted into the flexible net or mesh.

In an example, a tubular mesh can be soldered, melted, glued, or crimped onto an annular member. In an example, an annular member can have an inner ring and an outer ring, wherein a tubular mesh is fixed (e.g. soldered, melted, glued, or crimped) between the two rings. In an example, an annular member can comprise an inner ring or cylinder and an outer elastic band, wherein the tubular mesh is held between the inner and outer portions. In this example, an annular member can be centrally-located with respect to a proximal surface of the flexible net or mesh. In an example, an annular member can be centrally-located with respect to the longitudinal axis of the flexible net or mesh. In an example, an annular member can be a hub into which proximal ends of braided wires or tubes of the stent are bound or attached. In an example, an annular member can be off-axial with respect to the longitudinal axis of the flexible net or mesh.

In an example, an annular member can comprise two nested and/or concentric (inner and outer) cylinders, wherein the tubular mesh is pinched and/or crimped between the two cylinders. In an example, an annular member can comprise two nested and/or concentric (inner and outer) rings or bands, wherein the tubular mesh is pinched and/or crimped between the two rings or bands. In an example, an annular member can comprise two nested and/or concentric (inner and outer) cylinders, wherein the tubular mesh is melted or glued between the two cylinders. In an example, an annular member can comprise two nested and/or concentric (inner and outer) rings or bands, wherein the tubular mesh is melted or glued between the two rings or bands.

In an example, an annular member can be a catheter which extends through the proximal surface of a flexible net or mesh, wherein the catheter is detached and/or removed after embolic members and/or material has been inserted through the catheter into the interior or distal-facing concavity of the flexible net or mesh. In an example, a distal portion of the catheter used to deliver embolic members and/or material can extend through the proximal surface of a flexible net or mesh and be detached from the rest of the catheter after embolic members and/or material has been inserted through the catheter. In an example, an annular member can be attached to a catheter during delivery of embolic members and/or material, and then detached (e.g. by the application of electromagnetic energy) from the catheter after delivery of the embolic members and/or material.

In an example, an annular member can have an outer diameter which is between 5% and 20% of the diameter of the tubular mesh before the tubular mesh is radially constrained. In an example, an annular member can have an outer diameter which is between 10% and 33% of the diameter of the tubular mesh before the tubular mesh is radially constrained. In an example, an annular member can have an outer ring (or cylinder) with a first diameter and an inner ring (or cylinder) with a second diameter, wherein the tubular mesh is crimped or pinched between the outer ring (or cylinder) and inner ring (or cylinder), and wherein the first diameter is between 50% and 75% of the second diameter. In an example, an annular member can have an outer ring (or cylinder) with a first diameter and an inner ring (or cylinder) with a second diameter, wherein the tubular mesh is crimped or pinched between the outer ring (or cylinder) and inner ring (or cylinder), and wherein the first diameter is between 66% and 90% of the second diameter.

In an example, an annular member can comprise two nested rings, bands, or cylinders, wherein a section of the tubular mesh is inserted and held between the nested rings, bands, or cylinders. In an example, an annular member can comprise an outer ring, band, or cylinder and an inner ring, band, or cylinder, wherein a section of the tubular mesh is inserted and held between them. In an example, an annular member can comprise an outer ring, band, or cylinder and an inner ring, band, or cylinder, wherein one or both of the rings, bands, or cylinders are threaded. In an example, an annular member can comprise an outer ring, band, or cylinder and an inner ring, band, or cylinder, wherein one or both of the rings, bands, or cylinders has a helical thread. In an example, an annular member can comprise an outer ring, band, or cylinder and an inner ring, band, or cylinder, wherein one or both of the rings, bands, or cylinders has a helical thread to hold a section of the tubular mesh.

In an example, this device can further comprise a closure mechanism which closes an opening through an annular member. The closure mechanism can be closed after embolic members and/or material has been inserted into a flexible net or mesh. In an example, this closure mechanism can be selected from the group consisting of: valve; electric detachment mechanism; elastic ring or band; threaded mechanism; sliding cover; sliding plug; filament loop; and electromagnetic solenoid. In an example, a closure mechanism can be a leaflet valve. In an example, a closure mechanism can be a one-way valve. In an example, a valve can allow embolic members and/or material to enter a flexible net or mesh through an opening in an annular member, but not allow the embolic members and/or material to exit the net or mesh.

In an example, a tubular mesh can be made from a polymer. In an example, a tubular mesh can be woven or braided from polymer threads, filaments, yarns, or strips. In an example, a tubular mesh can be 3D printed. In an example, a flexible net or mesh can be made from a flexible polymer. In an example, a flexible net or mesh can be made from an elastic and/or stretchable polymer. In an example, a flexible net or mesh can be elastic and/or stretchable and can expand as it is filled with embolic members and/or material. In an example, a flexible net or mesh can be sufficiently flexible to conform to the shape of even an irregularly-shaped aneurysm sac as the net or mesh is filled with embolic members and/or material. In an example, a flexible net or mesh can be sufficiently flexible to conform to the shape of even an irregularly-shaped (e.g. non-spherical) aneurysm sac as the net or mesh is filled with embolic members and/or material. In an example, a tubular mesh can be made from one or more materials selected from the group consisting of: Dacron, elastin, hydroxy-terminated polycarbonate, methylcellulose, nylon, PDMS, polybutester, polycaprolactone, polyester, polyethylene terephthalate, polypropylene, polytetrafluoroethene, polytetrafluoroethylene, polyurethane, silicone, and silk.

In an example, a tubular mesh can be made from metal. In an example, a tubular mesh can be made from Nitinol. In an example, a tubular mesh can be a flexible metal mesh. In an example, a tubular mesh can be a braided metal mesh. In an example, a tubular mesh can be woven or braided from metal filaments, wires, or tubes. In an example, a tubular mesh can be made from shape-memory material. In an example, a tubular mesh can be made with both metal and polymer components.

In an example, openings or holes in a flexible net or mesh can be smaller than the size (e.g. diameter, width, and/or length) of embolic members and/or material which is inserted into the net or mesh so that the embolic members and/or material do not escape out of the net or mesh. In an example, openings or holes in a flexible net or mesh can less than half of the size (e.g. diameter, width, and/or length) of embolic members and/or material which is inserted into the net or mesh so that the embolic members and/or material do not escape out of the net or mesh. In an example, openings or holes in a flexible net or mesh can have a size which is less than half of the smallest diameter and/or width of embolic members and/or material which is inserted into the net or mesh so that the embolic members and/or material do not escape out of the net or mesh. In an example, openings or holes in a flexible net or mesh can have a size which less than half of the smallest length of embolic members and/or material which is inserted into the net or mesh so that the embolic members and/or material do not escape out of the net or mesh.

In an example, a tubular mesh can have hexagonal openings In an example, a tubular mesh with hexagonal openings can be made using 3D printing. In an example, a flexible metal tubular mesh with hexagonal openings can be made by 3D printing with liquid metal. In an example, a tubular mesh with hexagonal openings can be made by 3D printing with a polymer. In an example, a tubular mesh with hexagonal openings can be made by 3D printing with an elastomeric polymer. In an example, a tubular mesh with hexagonal openings can be made by 3D printing with a silicone-based polymer. In an example, a tubular mesh with hexagonal openings can be made by 3D printing with polydimethylsiloxane (PDMS).

In an example, a tubular mesh can have quadrilateral openings. In an example, a tubular mesh with quadrilateral openings can be made using 3D printing. In an example, a flexible metal tubular mesh with quadrilateral openings can be made by 3D printing with liquid metal. In an example, a tubular mesh with quadrilateral openings can be made by 3D printing with a polymer. In an example, a tubular mesh with quadrilateral openings can be made by 3D printing with an elastomeric polymer. In an example, a tubular mesh with quadrilateral openings can be made by 3D printing with a silicone-based polymer. In an example, a tubular mesh with quadrilateral openings can be made by 3D printing with polydimethylsiloxane (PDMS).

In an example, a tubular mesh can have circular openings. In an example, a tubular mesh with circular openings can be made using 3D printing. In an example, a flexible metal tubular mesh with circular openings can be made by 3D printing with liquid metal. In an example, a tubular mesh with circular openings can be made by 3D printing with a polymer. In an example, a tubular mesh with circular openings can be made by 3D printing with an elastomeric polymer. In an example, a tubular mesh with circular openings can be made by 3D printing with a silicone-based polymer. In an example, a tubular mesh with circular openings can be made by 3D printing with polydimethylsiloxane (PDMS).

In an example, a tubular mesh can be made with a cobalt chromium alloy. In an example, a tubular mesh can be made with a nickel-titanium alloy. In an example, a tubular mesh can comprise cobalt chromium alloy wires, filaments, or tubes. In an example, a tubular mesh can comprise nickel-titanium alloy wires, filaments, or tubes. In an example, a tubular mesh can comprise nitinol wires, filaments, or tubes. In an example, a tubular mesh can be made with nitinol. In an example, a tubular mesh can comprise platinum wires, filaments, or tubes. In an example, a tubular mesh can be made with platinum. In an example, a tubular mesh can comprise stainless steel wires, filaments, or tubes. In an example, a tubular mesh can be made with stainless steel. In an example, a tubular mesh can comprise tantalum wires, filaments, or tubes. In an example, a tubular mesh can be made with tantalum.

In an example, a mid-section of tubular mesh can be more flexible than the proximal and distal sections of the tubular mesh to predispose the tubular mesh to fold over itself at that mid-section to more easily form a dual-layer flexible net or mesh via inversion or eversion of the tubular mesh. In an example, a mid-section of tubular mesh can be more flexible than the proximal and distal sections of the tubular mesh to predispose the tubular mesh to fold over itself at that mid-section to more easily form a dual-layer bowl-shaped flexible net or mesh via inversion or eversion of the tubular mesh. In an example, a mid-section of tubular mesh can be less dense than the proximal and distal sections of the tubular mesh to predispose the tubular mesh to fold over itself at that mid-section to more easily form a dual-layer flexible net or mesh via inversion or eversion of the tubular mesh.

In an example, a mid-section of tubular mesh can be less dense than the proximal and distal sections of the tubular mesh to predispose the tubular mesh to fold over itself at that mid-section to more easily form a dual-layer bowl-shaped flexible net or mesh via inversion or eversion of the tubular mesh. In an example, a mid-section of tubular mesh can be made with a lower-durometer material than the proximal and distal sections of the tubular mesh to predispose the tubular mesh to fold over itself at that mid-section to more easily form a dual-layer flexible net or mesh via inversion or eversion of the tubular mesh. In an example, a mid-section of tubular mesh can be made with a lower-durometer material than the proximal and distal sections of the tubular mesh to predispose the tubular mesh to fold over itself at that mid-section to more easily form a dual-layer bowl-shaped flexible net or mesh via inversion or eversion of the tubular mesh.

In an example, a distal portion (e.g. the distal half) of a flexible net or mesh can have a lower durometer than the proximal portion (e.g. the proximal half) of the flexible net or mesh. In an example, a distal portion (e.g. the distal half) of a flexible net or mesh can be more flexible than the proximal portion (e.g. the proximal half) of the flexible net or mesh. In an example, a distal portion (e.g. the distal half) of a flexible net or mesh can be less dense than the proximal portion (e.g. the proximal half) of the flexible net or mesh. In an example, a distal portion (e.g. the distal half) of a flexible net or mesh can be more porous than the proximal portion (e.g. the proximal half) of the flexible net or mesh.

In an example, a flexible net or mesh can be folded and/or compressed as it is delivered through a catheter to an aneurysm sac. In an example, a flexible net or mesh can have radial folds as it is delivered through a catheter to an aneurysm sac. In an example, a flexible net or mesh can have longitudinal folds as it is delivered through a catheter to an aneurysm sac. In an example, a flexible net or mesh can have cross-sectional folds as it is delivered through a catheter to an aneurysm sac.

In an example, a flexible net or mesh can have a longitudinal axis which spans in a proximal-to-distal direction. Proximal can be defined as being closer to the point of entry into a person's body during delivery through the person's vasculature (in the catheter) to the aneurysm and closer to the aneurysm neck after insertion into the aneurysm sac. In this example, a tubular mesh is transformed into double-layer, distally-concave, bowl-shaped flexible net or mesh by a single annular member in a middle section (between the ends) of the tubular mesh which radially-constrains the middle of the tubular mesh, wherein the proximal portion of the mesh is everted distally over the distal portion of the mesh until it has a distally-concave shape. In this example, the distal circumference of the flexible net or mesh comprises two nested tubular openings.

Alternatively, a tubular mesh can be transformed into a single-layer, distally-concave, bowl-shaped flexible net or mesh by a single annular member which radially-constrains the proximal end of the tubular mesh. In an example, a tubular mesh can be transformed into single-layer, proximally-concave, bowl-shaped flexible net or mesh by a single annular member which radially-constrains the distal end of the tubular mesh.

In an example, a tubular mesh can be transformed into a double-layer, distally-concave, bowl-shaped flexible net or mesh by two annular members (a proximal annular member and a distal annular member) which radially-constrain the proximal and distal ends of the tubular mesh, wherein the distal portion of the tubular mesh is inverted proximally (e.g. folded proximally) until it has a distally-concave shape. In an example, the distal circumference of the flexible net or mesh can be a fold in the net or mesh. In an example, proximal and distal annular members can be aligned so that embolic members and/or material can be delivered through them into the distal-facing concavity of the double-layer bowl-shaped flexible net or mesh. In an example, both of the radially-constrained ends can project into the interior of flexible net or mesh. In an example, the proximal end can be inverted to project into the interior of bowl-shaped flexible net or mesh and the distal end is not.

In an example, a tubular mesh can be transformed into a single-layer ellipsoidal and/or generally globular flexible net or mesh by two annular members which radially-constrain the proximal and distal ends of the tubular mesh. In an example, both of these radially-constrained ends can be inverted to project into the interior of flexible net or mesh. In an example, the proximal end can be inverted to project into the interior of flexible net or mesh and the distal end can remain outside the interior of the flexible net or mesh. In an example, a tubular mesh is transformed into single-layer spherical flexible net or mesh by two annular members which radially-constrain the proximal and distal ends of the tubular mesh.

In an example, bound and/or inverted ends of a flexible net or mesh can both extend into the interior of a flexible net or mesh in a spherical, ellipsoidal, and/or globular configuration. In an example, a distal bound and/or inverted end of a flexible net or mesh can extend into the interior of a flexible net or mesh in a spherical, ellipsoidal, and/or globular configuration and a proximal bound and/or inverted end of the flexible net or mesh can extend outward from a flexible net or mesh in a spherical, ellipsoidal, and/or globular configuration. In an example, a proximal bound and/or inverted end of a flexible net or mesh can extend into the interior of a flexible net or mesh in a spherical, ellipsoidal, and/or globular configuration and a distal bound and/or inverted end of the flexible net or mesh can extend outward from a flexible net or mesh in a spherical, ellipsoidal, and/or globular configuration.

In an example, a tubular mesh can be made from polycarbonate urethane (PCU). In an example, a tubular mesh can be made from polydimethylsiloxane (PDMS). In an example, a tubular mesh can be made from polyesters. In an example, a tubular mesh can be made from polyether block amide (PEBA). In an example, a tubular mesh can be made from polyetherether ketone (PEEK). In an example, a tubular mesh can be made from polyethylene. In an example, a tubular mesh can be made from polyethylene glycol (PEG). In an example, a tubular mesh can be made from polyethylene terephthalate (PET).

In an example, a tubular mesh can be made from polyglycolic acid (PGA). In an example, a tubular mesh can be made from polylactic acid (PLA). In an example, a tubular mesh can be made from poly-N-acetylglucosamine (PNAG). In an example, a tubular mesh can be made from polyolefin. In an example, a tubular mesh can be made from polyoleandlena. In an example, a tubular mesh can be made from polypropylene. In an example, a tubular mesh can be made from polytetrafluoroethylene (PTFE). In an example, a tubular mesh can be made from polyurethane (PU). In an example, a tubular mesh can be made from polywanacrakor. In an example, a tubular mesh can be made from polyvinyl alcohol (PVA). In an example, a tubular mesh can be made from polyvinyl pyrrolidone (PVP).

In an example, a tubular mesh from which a flexible net or mesh is formed can be tapered. In an example, the distal end of a tubular mesh can have a smaller diameter than the proximal end of the tubular mesh. In an example, the distal end of a tubular mesh can have a larger diameter than the proximal end of the tubular mesh. In an example, a tubular mesh from which a flexible net or mesh is formed can have differential flexibility. In an example the distal portion of a tubular mesh can have a first level of flexibility and the proximal portion of the tubular mesh can have a second level of flexibility, wherein the first level is less than the second level. In an example the distal portion of a tubular mesh can have a first level of flexibility and the proximal portion of the tubular mesh can have a second level of flexibility, wherein the first level is greater than the second level.

In an example, a tubular mesh from which a flexible net or mesh is formed can have differential porosity. In an example the distal portion of a tubular mesh can have a first porosity level and the proximal portion of the tubular mesh can have a second porosity level, wherein the first level is less than the second level. In an example the distal portion of a tubular mesh can have a first porosity level and the proximal portion of the tubular mesh can have a second porosity level, wherein the first level is greater than the second level. In an example, a tubular mesh from which a flexible net or mesh is formed can have differential durometer. In an example the distal portion of a tubular mesh can have a first durometer level and the proximal portion of the tubular mesh can have a second durometer level, wherein the first level is less than the second level. In an example the distal portion of a tubular mesh can have a first durometer level and the proximal portion of the tubular mesh can have a second durometer level, wherein the first level is greater than the second level.

In an example, the width of a flexible net or mesh in a bowl-shaped configuration can be larger than the width of the aneurysm neck. In an example, the circumference of a flexible net or mesh in a bowl-shaped configuration can be larger than the circumference of the aneurysm neck. In an example, the width of a flexible net or mesh in a bowl-shaped configuration can be at least 10% larger than the width of the aneurysm neck. In an example, the circumference of a flexible net or mesh in a bowl-shaped configuration can be at least 10% larger than the circumference of the aneurysm neck. In an example, the width of a flexible net or mesh in a bowl-shaped configuration can be at least 90% of the maximum width of the aneurysm sac (parallel to the aneurysm neck). In an example, the circumference of a flexible net or mesh in a bowl-shaped configuration can be at least 90% of the circumference of the maximum circumference of the aneurysm sac (parallel to the aneurysm neck). In an example, a flexible net or mesh can function as a neck bridge, reducing or completely blocking blood flow from the parent vessel into the aneurysm sac.

In an example, a flexible net or mesh formed from a tubular mesh can have a generally-hemispherical shape after a tubular mesh has been radially-constrained by one or more annular members. In an example, a flexible net or mesh formed from a tubular mesh can have a generally globular and/or spherical shape after a tubular mesh has been radially-constrained by one or more annular members. In an example, a flexible net or mesh formed from a tubular mesh can have an ellipsoidal or oval shape after a tubular mesh has been radially-constrained by one or more annular members. In an example, a flexible net or mesh formed from a tubular mesh can have a disk shape after a tubular mesh has been radially-constrained by one or more annular members.

In an example, a flexible net or mesh formed from a tubular mesh can have the shape of a paraboloid-of-revolution (e.g. a paraboloid revolved around a left or right vertical edge) after a tubular mesh has been radially-constrained by one or more annular members. In an example, a flexible net or mesh formed from a tubular mesh can comprise a carlavian curve shape after a tubular mesh has been radially-constrained by one or more annular members. In an example, a flexible net or mesh formed from a tubular mesh can have a toroidal shape after a tubular mesh has been radially-constrained by one or more annular members. In an example, a flexible net or mesh formed from a tubular mesh can have a half-toroidal shape (e.g. a sliced bagel shape) after a tubular mesh has been radially-constrained by one or more annular members.

In an example, the distal end of a tubular mesh can be radially-constrained by a distal annular member and the proximal end of a tubular mesh can be radially-constrained by a proximal annular member to form a generally-globular, spherical, and/or ellipsoidal flexible net or mesh which is inserted into an aneurysm sac. In an example, the distal end of a tubular mesh can be radially-constrained by a distal annular member and the proximal end of a tubular mesh can be radially-constrained by a proximal annular member to form a generally-globular, spherical, and/or ellipsoidal shape, wherein the distal portion is then inverted and/or folded to create a two-layer bowl-shaped flexible net or mesh which is inserted into an aneurysm sac. In an example, both the distal end of a tubular mesh and the proximal end of a tubular mesh can be radially-constrained by a proximal annular member to form a two-layer bowl-shaped flexible net or mesh which is inserted into an aneurysm sac.

In an example a flexible net or mesh can be a two-layer bowl-shaped mesh with a distally-concave proximal layer and a distally-concave distal layer. In an example a flexible net or mesh can be a two-layer bowl-shaped mesh with a distally-concave proximal layer and a distally-concave distal layer, wherein the distance between the proximal and distal layers is greater in a radially-central portion of the flexible net or mesh than in radially-peripheral portions of the flexible net or mesh. In an example a flexible net or mesh can be a two-layer bowl-shaped mesh with a proximal layer and a distal layer, wherein the proximal layer has a uniform distal-facing concavity, but the distal layer has locally-concave and locally-convex portions. In an example, the radially-central portion of the distal layer is locally-convex and the radially-peripheral portions of the distal layer are locally-concave. In an example, the radially-central portion of the distal layer is less distally-concave than the radially-peripheral portions of the distal layer.

In an example, embolic members and/or material which is inserted into the flexible net or mesh can be microspheres or microballs. In an example, embolic members and/or material inserted into the flexible net or mesh can be microsponges. In an example, embolic members and/or material inserted into the flexible net or mesh can be pieces of foam. In an example, embolic members and/or material inserted into the flexible net or mesh can be microbeads. In an example, embolic members and/or material inserted into the flexible net or mesh can be pieces of hydrogel. In an example, embolic members and/or material inserted into the flexible net or mesh can be metal embolic coils. In an example, embolic members and/or material inserted into the flexible net or mesh can be embolic ribbons. In an example, embolic members and/or material inserted into the flexible net or mesh can be yarns or filaments. In an example, embolic members and/or material can be polymer strands or coils. In an example, accumulation of embolic members and/or material in an aneurysm sac can compress a flexible net or mesh from a spherical, ellipsoidal, and/or globular configuration into a hemispherical, bowl-shaped, and/or distally-concave configuration by pressing against the distal surface of the flexible net or mesh.

In an example, embolic members and/or material inserted into the flexible net or mesh can be microspheres or microballs connected by a longitudinal wire, cord, and/or filament (e.g. in a “string-of-pearls” configuration). In an example, embolic members and/or material inserted into the flexible net or mesh can be microsponges connected by a longitudinal wire, cord, and/or filament (e.g. in a “string-of-pearls” configuration). In an example, embolic members and/or material inserted into the flexible net or mesh can be pieces of foam connected by a longitudinal wire, cord, and/or filament (e.g. in a “string-of-pearls” configuration). In an example, embolic members and/or material inserted into the flexible net or mesh can be microbeads connected by a longitudinal wire, cord, and/or filament (e.g. in a “string-of-pearls” configuration).

In an example, embolic members and/or material inserted into the flexible net or mesh can be pieces of hydrogel connected by a longitudinal wire, cord, and/or filament (e.g. in a “string-of-pearls” configuration). In an example, embolic members and/or material inserted into the flexible net or mesh can be embolic coils connected by a longitudinal wire, cord, and/or filament (e.g. in a “string-of-pearls” configuration). In an example, embolic members and/or material inserted into the flexible net or mesh can be embolic ribbons connected by a longitudinal wire, cord, and/or filament (e.g. in a “string-of-pearls” configuration). In an example, embolic members and/or material inserted into the flexible net or mesh can be yarns or filaments connected by a longitudinal wire, cord, and/or filament (e.g. in a “string-of-pearls” configuration).

In an example, embolic members and/or material inserted into the flexible net or mesh can be liquid which congeals and/or solidifies. In an example, embolic members and/or material inserted into the flexible net or mesh can be a polymer which congeals and/or solidifies. In an example, embolic members and/or material inserted into the flexible net or mesh can be a liquid embolic material. In an example, embolic members and/or material inserted into the flexible net or mesh can be hydrogel material. In an example, embolic members and/or material inserted into the flexible net or mesh can be congealing adhesive material. In an example, accumulation of embolic members and/or material in an aneurysm sac can compress a flexible net or mesh from a spherical, ellipsoidal, and/or globular configuration to a hemispherical, bowl-shaped, and/or distally-concave configuration by pressing against the distal surface of the flexible net or mesh.

In an example, embolic members and/or material which is inserted through an annular member into a flexible net or mesh can be one or more mesh ribbons. In an example, embolic members and/or material which is inserted through an annular member into a flexible net or mesh can be one or more wire mesh ribbons. In an example, embolic members and/or material which is inserted through an annular member into a flexible net or mesh can be one or more polymer mesh ribbons. In an example, embolic members and/or material which is inserted through an annular member into a flexible net or mesh can be one or more undulating and/or sinusoidal ribbons. In an example, embolic members and/or material which is inserted through an annular member into a flexible net or mesh can be one or more double-layer mesh ribbons.

In an example, embolic members and/or material can be made with a cobalt chromium alloy. In an example, embolic members and/or material can be made with a nickel-titanium alloy. In an example, embolic members and/or material can be cobalt chromium alloy coils or ribbons. In an example, embolic members and/or material can be nickel-titanium alloy coils or ribbons. In an example, embolic members and/or material can be nitinol coils or ribbons. In an example, embolic members and/or material can be made with nitinol. In an example, embolic members and/or material can be platinum coils or ribbons. In an example, embolic members and/or material can be made with platinum. In an example, embolic members and/or material can be stainless steel coils or ribbons. In an example, embolic members and/or material can be made with stainless steel. In an example, embolic members and/or material can be tantalum coils or ribbons. In an example, embolic members and/or material can be made with tantalum.

In an example, embolic members and/or material can be pushed through a catheter into a flexible net or mesh by a pusher wire and/or plug. In an example, liquid embolic material (which congeals after insertion into the net or mesh) can be pushed through a catheter into a flexible net or mesh by fluid pressure. In an example, embolic members can be pushed into a flexible net or mesh by a flow of liquid (e.g. saline solution), wherein the embolic members are retained in the flexible net or mesh and the saline solution escapes out of openings in the flexible net or mesh. In an example, embolic members and/or material can be pushed through a catheter into a flexible net or mesh by a conveyer belt mechanism. In an example, embolic members and/or material can be pushed through a catheter into a flexible net or mesh by a rotating helical delivery mechanism.

In an example, embolic members which are inserted into a net or mesh can be embolic coils or ribbons. In an example, embolic members which are inserted into a net or mesh can be pieces of foam or gel (such as hydrogel). In an example, embolic members which are inserted into a net or mesh can be microballs or microspheres. In an example, embolic members which are inserted into a net or mesh can be microsponges. In an example, embolic members which are inserted into a net or mesh can be filaments or yarns. In an example, liquid embolic material can be inserted into a net or mesh.

In an example, embolic members which are inserted into a net or mesh can be selected from the group consisting of: pieces of gel; pieces of foam; and micro-sponges. In an example, embolic members which are inserted into a net or mesh can be pieces of gel, such as hydrogel. In an example, embolic members which are inserted into a net or mesh can be pieces of foam. In an example, embolic members which are inserted into a net or mesh can be micro-sponges. In an example, embolic members which are inserted into a net or mesh can be microscale gel balls. In an example, embolic members which are inserted into a net or mesh can be microscale foam balls. In an example, embolic members which are inserted into a net or mesh can be microscale sponge balls. In an example, embolic members which are inserted into a net or mesh can be microscale gel polyhedrons. In an example, embolic members which are inserted into a net or mesh can be microscale foam polyhedrons. In an example, embolic members which are inserted into a net or mesh can be microscale sponge polyhedrons.

In an example, embolic members which are inserted into a net or mesh can have generally spherical or globular shapes. In an example, embolic members which are inserted into a net or mesh can have generally prolate spherical, ellipsoidal, or ovaloid shapes. In an example, embolic members which are inserted into a net or mesh can have apple, barrel, or pair shapes. In an example, embolic members which are inserted into a net or mesh can have torus or ring shapes. In an example, embolic members which are inserted into a net or mesh can have disk or pancake shapes. In an example, embolic members which are inserted into a net or mesh can have peanut or hour-glass shapes. In an example, embolic members which are inserted into a net or mesh can be polyhedrons comprised of hexagonal surfaces. In an example, embolic members which are inserted into a net or mesh can be polyhedrons comprised of quadrilateral surfaces. In an example, embolic members which are inserted into a net or mesh can be polyhedrons comprised of triangular surfaces.

In an example, an embolic member can have a shape which is selected from the group consisting of: apple-shaped, barrel-shaped, bulbous, convex, ellipsoidal, globular, oblate spheroid, ovaloid, prolate-spheroid-shaped, spherical, and truncated-sphere-shaped. In an example, an embolic member can have a shape which is selected from the group consisting of: bowl-shaped, concave, hemispherical, and paraboloid of revolution. In an example, an embolic member can have a shape which is selected from the group consisting of: cubic, hexagon-shaped, hexahedron, octagon-shaped, octahedron, pentagonal-shaped, polyhedron-shaped, pyramidal, rectangular, square, and tetrahedronal.

In an example, embolic members which are inserted into a net or mesh can have a (diameter) size within the range of 0.5 to 2 millimeters. In an example, embolic members which are inserted into a net or mesh can have a (diameter) size within the range of 1 to 5 millimeters. In an example, embolic members which are inserted into a net or mesh can have a (diameter) size within the range of 2 to 10 millimeters. In an example, embolic members which are inserted into a net or mesh can have a (diameter) size within the range of 5 to 20 millimeters. In an example, embolic members which are inserted into a net or mesh can have a (diameter) size within the range of 0.5 to 2 microns. In an example, embolic members which are inserted into a net or mesh can have a (diameter) size within the range of 1 to 5 microns. In an example, embolic members which are inserted into a net or mesh can have a (diameter) size within the range of 2 to 10 microns. In an example, embolic members which are inserted into a net or mesh can have a (diameter) size within the range of 5 to 20 microns.

In an example, between 5 and 20 embolic members can be inserted into a net or mesh. In an example, between 10 and 50 embolic members can be inserted into a net or mesh. In an example, between 20 and 100 embolic members can be inserted into a net or mesh. In an example, between 50 and 500 embolic members can be inserted into a net or mesh.

In an example, embolic members which are inserted into a net or mesh can expand in size within the net or mesh. In an example, embolic members can have a first (average) size while being delivered to an aneurysm sac via a micro-catheter and a second (average) size after expansion within the aneurysm sac, wherein the second (average) size is 10% to 50% larger than the first (average) size. In an example, embolic members can have a first (average) size while being delivered to an aneurysm sac via a micro-catheter and a second (average) size after expansion within the aneurysm sac, wherein the second (average) size is 40% to 100% larger than the first (average) size. In an example, embolic members can have a first (average) size while being delivered to an aneurysm sac via a micro-catheter and a second (average) size after expansion within the aneurysm sac, wherein the second (average) size is more than twice the first (average) size.

In an example, embolic members can self-expand within a net or mesh after they are released from a delivery catheter. In an example, embolic members can swell upon hydration from interaction with blood or other body fluid. In an example, embolic members can be expanded within the net or mesh by one or more mechanisms selected from the group consisting of: expansion due to interaction with body fluid; expansion due to application of thermal energy; expansion due to exposure to a chemical agent; and expansion due to exposure to light energy. In an example, embolics can expand by a factor of 2-5 times. In an example, embolics can expand by a factor of 4-10 times. In an example, embolics can expand by a factor of more than 10 times. In an example, embolic members can expand to a sufficiently-large size that they cannot escape from the net or mesh after insertion into the net or mesh.

In an example, three-dimensional embolic members which are inserted into a net or mesh can be soft and compressible. In an example, three-dimensional embolic members which are inserted into a net or mesh can have a durometer less than 50. In an example, three-dimensional embolic members which are inserted into a net or mesh can have an average durometer within the range of 10 to 30. In an example, three-dimensional embolic members which are inserted into a net or mesh can have an average durometer within the range of 25 to 50. In an example, three-dimensional embolic members which are inserted into a net or mesh can have an average durometer which is less than 70.

In an example, embolic members which are inserted into a net or mesh can be made from a polymer. In an example, embolic members which are inserted into a net or mesh can be made from an elastomeric polymer. In an example, embolic members which are inserted into a net or mesh can be made from a silicone-based polymer. In an example, embolic members which are inserted into a net or mesh can be made from polydimethylsiloxane (PDMS).

In an example, an embolic member can further comprise one or more layers made with different materials. In an example, an inner layer of an embolic member can be made from a first material and an outer layer of an embolic member can be made from a second material. In an example, an inner layer of an embolic member can be made from a first material with a first durometer and an outer layer of an embolic member can be made from a second material with a second durometer, wherein the second durometer is less than the first durometer. In an example, an embolic member can have an outer layer which is adhesive. In an example, an embolic member can have an outer layer with an adhesive property which is activated by application of electromagnetic and/or thermal energy. In an example, an embolic member can have an outer layer with an adhesive property which is activated by interaction with blood.

In an example, there can be a first average durometer of embolic members which are inserted into the net or mesh at a first time and a second average durometer of embolic members which are inserted into the net or mesh at a second time, wherein the second average durometer is greater than the first average durometer. In an example, there can be a first average durometer of embolic members which are inserted into the net or mesh at a first time and a second average durometer of embolic members which are inserted into the net or mesh at a second time, wherein the second average durometer is less than the first average durometer.

In an example, there can be a first average length of longitudinal strands between proximal pairs of embolic members which are inserted into a net or mesh at a first time, a second average length of longitudinal strands between proximal pairs of embolic members which are inserted into the net or mesh at a second time, and the second average length can be greater than the first average length. In an example, there can be a first average length of longitudinal strands between proximal pairs of embolic members which are inserted into a net or mesh at a first time, a second average length of longitudinal strands between proximal pairs of embolic members which are inserted into the net or mesh at a second time, and the second average length can be less than the first average length.

In an example, there can be a first set of embolic members which are inserted into a net or mesh at a first time and a second set of embolic members which are inserted into the net or mesh at a second time, wherein the second set of embolic members are closer together than the first set of embolic members. In an example, there can be a first set of embolic members which are inserted into a net or mesh at a first time and a second set of embolic members which are inserted into the net or mesh at a second time, wherein the first set of embolic members are closer together than the second set of embolic members. In an example, there can be a longitudinal series of embolic members connected by one or more longitudinal strands which is inserted into a net or mesh within an aneurysm sac, wherein embolic members in the longitudinal series are progressively closer to each other moving along the length of the series in a distal to proximal manner. In an example, there can be a longitudinal series of embolic members connected by one or more longitudinal strands which is inserted into a net or mesh within an aneurysm sac, wherein embolic members in the longitudinal series are progressively farther from each other moving along the length of the series in a distal to proximal manner.

In an example, embolic members which are inserted into the net or mesh at a first time can have first shapes, embolic members which are inserted into the net or mesh at a second time can have second shapes, and the second shape can be different than the first shape. In an example, embolic members which are inserted into the net or mesh at a first time can be made with a first (combination of) material, embolic members which are inserted into the net or mesh at a second time can be made with a second (combination of) material, and the second (combination of) material can be different from the first (combination of) material. In an example, embolic members which are inserted into the net or mesh at a first time can be made with a first (combination of) material, embolic members which are inserted into the net or mesh at a second time can be made with a second (combination of) material, and the second (combination of) material can be more flexible, elastic, and/or compliant than the first (combination of) material.

In an example, embolic members which are inserted into the net or mesh at a first time can be made with a first (combination of) material, embolic members which are inserted into the net or mesh at a second time can be made with a second (combination of) material, and the second (combination of) material can have a lower durometer than the first (combination of) material. In an example, embolic members which are inserted into the net or mesh at a first time can be made with a first (combination of) material, embolic members which are inserted into the net or mesh at a second time can be made with a second (combination of) material, and the second (combination of) material can be less flexible, elastic, and/or compliant than the first (combination of) material. In an example, embolic members which are inserted into the net or mesh at a first time can be made with a first (combination of) material, embolic members which are inserted into the net or mesh at a second time can be made with a second (combination of) material, and the second (combination of) material can have a higher durometer than the first (combination of) material.

In an example, there can be a first average size of embolic members which are inserted into the net or mesh at a first time, a second average size of embolic members which are inserted into the net or mesh at a second time, and the second average size can be greater than the first average size. In an example, there can be a first average size of embolic members which are inserted into the net or mesh at a first time, a second average size of embolic members which are inserted into the net or mesh at a second time, and the second average size can be less than the first average size.

In an example, a net or mesh can be delivered into an aneurysm sac via a catheter and/or delivery tube. In an example, a plurality of embolic members can be delivered into the net or mesh via the same catheter and/or delivery tube. In an example, a net or mesh can be delivered into an aneurysm sac via a first catheter and/or delivery tube and a plurality of embolic members can be delivered into the net or mesh via a second catheter and/or delivery tube.

In an example, embolic members can be made from ethylene vinyl alcohol (EVA). In an example, embolic members can be made from polyolefin. In an example, embolic members can be made from fibrinogen. In an example, embolic members can be made from polylactic acid (PLA). In an example, embolic members can be made from polyethylene terephthalate (PET). In an example, embolic members can be made from steel (e.g. stainless steel). In an example, embolic members can be made from methylcellulose.

In an example, embolic members can be made from acrylic. In an example, embolic members can be made from polyethylene glycol (PEG). In an example, embolic members can be made from silk. In an example, embolic members can be made from alginate. In an example, embolic members can be made from gold. In an example, embolic members can be made from polyethylene. In an example, embolic members can be made from polyoleandlena. In an example, embolic members can be made from tantalum. In an example, embolic members can be made from cobalt-chrome alloy (cobalt chromium).

In an example, embolic members can be made from polyetherether ketone (PEEK). In an example, embolic members can be made from polywanacrakor. In an example, embolic members can be made from thermoplastic elastomer. In an example, embolic members can be made from polycarbonate urethane (PCU). In an example, embolic members can be made from water-soluble synthetic polymer. In an example, embolic members can be made from collagen. In an example, embolic members can be made from polyvinyl alcohol (PVA).

In an example, embolic members can be made from titanium. In an example, embolic members can be made from polyether block amide (PEBA). In an example, embolic members can be made from radiopaque material. In an example, embolic members can be made from copolymer. In an example, embolic members can be made from polyvinyl pyrrolidone (PVP). In an example, embolic members can be made from polydimethylsiloxane (PDMS). In an example, embolic members can be made from zirconium-based alloy. In an example, embolic members can be made from polyesters. In an example, embolic members can be made from hydrogel. In an example, embolic members can be made from silicone. In an example, embolic members can be made from nitinol (or other nickel titanium alloy).

In an example, embolic members can be made from polyglycolic acid (PGA). In an example, embolic members can be made from small intestinal submucosa. In an example, embolic members can be made from nylon. In an example, embolic members can be made from polypropylene. In an example, embolic members can be made from platinum. In an example, embolic members can be made from polyurethane (PU). In an example, embolic members can be made from tungsten. In an example, embolic members can be made from fibrin.

In an example, embolic members can be made from poly-N-acetylglucosamine (PNAG). In an example, embolic members can be made from latex. In an example, embolic members can be made from fibronectin. In an example, embolic members can be made from palladium. In an example, embolic members can be made from polytetrafluoroethylene (PTFE). In an example, embolic members can be made from gelatin.

In an example, a selected quantity, series, length, and/or volume of embolic members can be selectively dispensed and/or detached into the net or mesh in situ by a mechanism selected from the group consisting of: breaking a connection between embolic members in a series of embolic members; cutting a connection between embolic members in a series of embolic members (e.g. with a cutting edge or laser); dissolving a connection between embolic members in a series of embolic members (e.g. with thermal energy or a chemical); electrolytic mechanism; hydraulic mechanism; injecting a flow of embolic members suspended in a liquid or gel into a net or mesh; melting a connection between embolic members in a series of embolic members (e.g. with thermal or light energy); progressing embolic members into a net or mesh via a conveyor belt (e.g. chain-based conveyor); progressing embolic members into a net or mesh via a helical conveyor (e.g. with an Archimedes' screw); pushing embolic members into a net or mesh using the force of a liquid flow; pusher rod and/or plunger; release detachment mechanism; and thermal detachment mechanism.

In an example, embolic members can differ among themselves with respect to one or more characteristics selected from the group consisting of: porosity, shape, size, material, composition, coating, radiopacity, strength, stiffness, and type. In an example, a plurality of embolic members can be delivered into a net or mesh in a linear (longitudinal) array or series of inter-connected embolic members. In an example, a plurality of embolic members can be delivered into a net or mesh in a linear (longitudinal) array of connected embolic members, wherein this linear array can be cut, separated, and/or detached in situ (in a remote manner) at one or more selected locations by the user of the device in order to deliver a selected quantity, length, or volume or embolic members. In an example, a plurality of embolic members can be delivered into a net or mesh in a planar array of inter-connected embolic members. In an example, a plurality of embolic members can be delivered into a net or mesh in a three-dimensional array of inter-connected embolic members.

In an example, a series of embolic members can be delivered into a net or mesh, wherein successive embolic members in the series are closer together. In an example, a series of embolic members can be delivered into a net or mesh, wherein embolic members in the series are progressively closer together (as one progresses along the series in a distal to proximal manner). In an example, a series of embolic members can be delivered into a net or mesh, wherein successive embolic members in the series are farther apart from each other. In an example, a series of embolic members can be delivered into a net or mesh, wherein embolic members in the series are progressively farther apart (as one progresses along the series in a distal to proximal manner).

In an example, a series of embolic members can be delivered into a net or mesh, wherein successive embolic members in the series decrease in durometer. In an example, a series of embolic members can be delivered into a net or mesh, wherein embolic members in the series have progressively lower durometer values (as one progresses along the series in a distal to proximal manner). In an example, a series of embolic members can be delivered into a net or mesh, wherein successive embolic members in the series increase in durometer. In an example, a series of embolic members can be delivered into a net or mesh, wherein embolic members in the series have progressively higher durometer values (as one progresses along the series in a distal to proximal manner).

In an example, a series of embolic members can be delivered into a net or mesh, wherein successive embolic members in the series are made of different materials. In an example, a series of embolic members can be delivered into a net or mesh, wherein successive embolic members in the series are made of different materials, wherein these materials differ in porosity. In an example, a series of embolic members can be delivered into a net or mesh, wherein successive embolic members in the series are made of different materials, wherein these materials differ in radiopacity. In an example, a series of embolic members can be delivered into a net or mesh, wherein successive embolic members in the series are made of different materials, wherein these materials differ in stiffness. In an example, a series of embolic members can be delivered into a net or mesh, wherein successive embolic members in the series are made of different materials, wherein these materials differ in durometer.

In an example, a series of embolic members can be delivered into a net or mesh, wherein successive embolic members in the series decrease in porosity. In an example, a series of embolic members can be delivered into a net or mesh, wherein embolic members in the series become progressively less porous (as one progresses along the series in a distal to proximal manner). In an example, a series of embolic members can be delivered into a net or mesh, wherein successive embolic members in the series increase in porosity. In an example, a series of embolic members can be delivered into a net or mesh, wherein embolic members in the series become progressively more porous (as one progresses along the series in a distal to proximal manner).

In an example, a series of embolic members can be delivered into a net or mesh, wherein successive embolic members in the series differ in shape. In an example, a series of embolic members can be delivered into a net or mesh, wherein successive embolic members in the series differ in their degree of convexity. In an example, a series of embolic members can be delivered into a net or mesh, wherein successive embolic members in the series differ in their degree of concavity.

In an example, a series of embolic members can be delivered into a net or mesh, wherein successive embolic members in the series decrease in size. In an example, a series of embolic members can be delivered into a net or mesh, wherein embolic members in the series become progressively smaller (as one progresses along the series in a distal to proximal manner). In an example, a series of embolic members can be delivered into a net or mesh, wherein successive embolic members in the series increase in size. In an example, a series of embolic members can be delivered into a net or mesh, wherein embolic members in the series become progressively larger (as one progresses along the series in a distal to proximal manner).

In an example, embolic members can be soft, compressible members such as microsponges or blobs of gel. In an example, embolic members can be made from sponge, foam, or gel. In an example, embolic members can be hard, uncompressible members such as hard polymer spheres or beads. In an example, embolic members can be made from one or more materials selected from the group consisting of: cellulose, collagen, acetate, alginic acid, carboxy methyl cellulose, chitin, collagen glycosaminoglycan, divinylbenzene, ethylene glycol, ethylene glycol dimethylmathacrylate, ethylene vinyl acetate, hyaluronic acid, hydrocarbon polymer, hydroxyethylmethacrylate, methlymethacrylate, polyacrylic acid, polyamides, polyesters, polyolefins, polysaccharides, polyurethane, polyvinyl alcohol, silicone, urethane, and vinyl stearate.

In an example, embolic members can have a shape selected from the group consisting of: ball or sphere, ovoid, ellipsoid, and polyhedron. In an example, embolic members can have a Shore OO value, indicative of softness or hardness, within a range of 5 to about 50. In an example, embolic members can have a diameter or like size within a range of 50 micrometers to 2000 micrometers. In an example, differently-sized embolic members can be used. In an example two or more different sizes of embolic members can be inserted into a net or mesh to occlude an aneurysm. In an example, embolic members can include small balls and large balls. In an example, it may be advantageous to first fill a net or mesh with larger balls and then continue filling the net or mesh with smaller balls. In another example, it may be advantageous to first fill a net or mesh with smaller balls and then continue filling the net or mesh with larger balls.

In an example, an intrasaccular aneurysm occlusion device can be filled with a “string of pearls” string (or wire) connected sequence of embolic members. In an example, an intrasaccular aneurysm occlusion device can include a series of embolic members which are connected by a strand. In an example, a device can include a string of pearls” series of embolic members which are linked by a strand (e.g. a thin flexible member). In an example, a device can include a string of pearls” series of embolic members which are centrally linked by a strand (e.g. a thin flexible member). In an example, a “string of pearls” string-or-wire connected sequence of embolic members can comprise a plurality of embolic members which are separate from each other, but pair-wise connected to each other by at least one string or wire. In an example, a plurality of members can be unevenly-spaced along the longitudinal axis of a flexible member. In an example, uneven spacing of the embolic members can be selected based on the size and shape of an aneurysm to be occluded. In an example, the distances between embolic members can vary. In an example, the space between embolic members can differ for occlusion of narrow-neck aneurysms vs. wide-neck aneurysms. In an example, distances between embolic members can become progressively shorter in a distal to proximal direction.

In an example, a line which connects embolic members can be a wire, spring, or chain. In an example, a connecting line can be a string, thread, band, fiber, or suture. In an example, embolic members can be centrally connected to each other by a connecting line. In an example, the centroids of embolic members can be connected by a connecting line. In an example, expanding arcuate embolic members can slide (e.g. up or down) along a connecting line. In an example, embolic members can slide along a connecting line, but only in one direction. In an example, a connecting line can have a ratchet structure which allows embolic members to slide closer to each other but not slide further apart. In an example, this device can further comprise a locking mechanism which stops embolic members from sliding along a connecting line. In an example, application of electromagnetic energy to a connecting line can fuse the line with the embolic members and stop them from sliding, effectively locking them in proximity to each other.

In an example, embolic members can be conveyed through a lumen to an aneurysm in a fluid flow, wherein the fluid escapes out from a net or mesh and the embolic members are retained within the net or mesh. In an example, embolic members can be conveyed through a lumen to an aneurysm by means of a moving belt or wire loop. In an example, embolic members can be conveyed through a lumen to an aneurysm by means of an Archimedes screw.

In an example, a flexible net or mesh can self-expand to a first extent after being released from a catheter into an aneurysm sac. In an example, the flexible net or mesh can further expand, to a second extent, due to pressure from the accumulation of embolic members and/or embolic material within its interior and/or distal-facing concavity. Other example variations discussed elsewhere in this disclosure or in priority-linked disclosures can also be applied to this example where relevant.

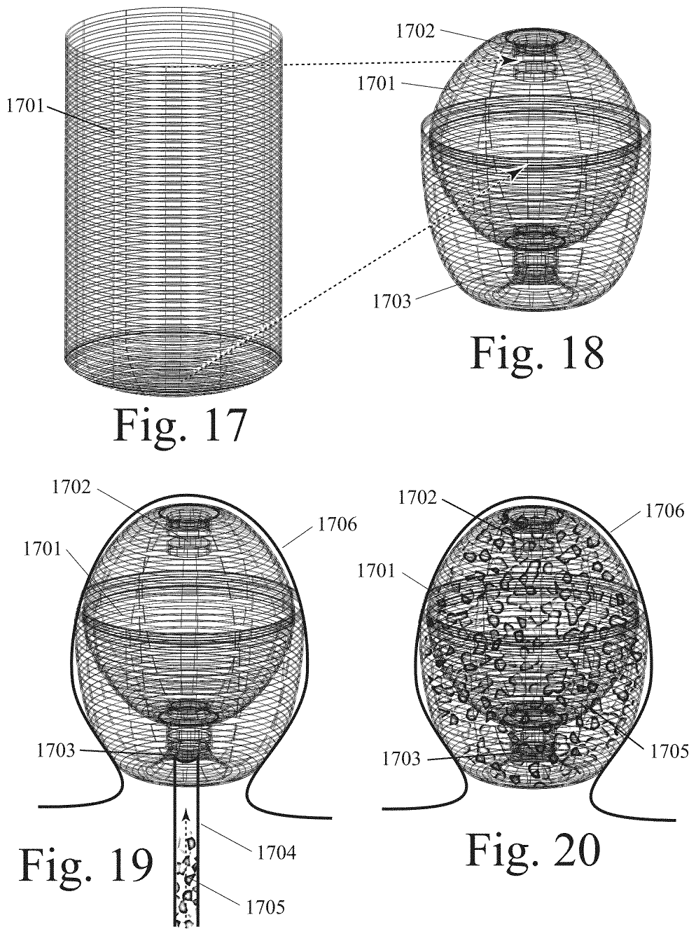

FIGS. 17 through 20 show four views, at different times, of the formation and deployment of another example of an intrasacular aneurysm occlusion device comprising: at least one annular member (in this example, mid-section annular member 1703 and distal annular member 1702), wherein an annular member is selected from the group consisting of a ring, a band, a cylinder, a tube, and a catheter; a flexible net or mesh, wherein the flexible net or mesh has a spherical, ellipsoidal, generally-globular, hemispherical, and/or bowl-shaped first configuration when it is formed by encircling, pinching, inverting, and/or everting a tubular mesh 1701 at one or more longitudinal locations using the at least one annular member; wherein the flexible net or mesh has a radially-compressed second configuration for delivery through a catheter 1704 into an aneurysm sac 1706; and wherein the flexible net or mesh is inserted and expanded within the aneurysm sac; and embolic members and/or embolic material 1705 which is inserted into the interior and/or the distal-facing concavity of the flexible net or mesh through one or more of the annular members. In this example, there are two annular members: a mid-section annular member which radially constrains a mid-section of the tubular mesh; and a distal annular member which radially constrains the distal end of the tubular mesh. In this example, the flexible net or mesh has a compound shape when it is first formed from the tubular mesh, wherein the compound shape is a globular shape inside the concavity of a bowl shape.

FIG. 17 introduces tubular mesh 1701 which is used to make the intrasacular aneurysm occlusion device. FIG. 18 shows two annular members: a mid-section annular member 1703 which radially-constrains a mid-section of the tubular mesh; and a distal annular member 1702 which radially constrains the distal end of the tubular mesh. The proximal portion of the mesh is everted over the globular distal portion of the mesh, creating a compound “ball in a bowl” shape as shown in FIG. 18. FIG. 19 shows this flexible mesh after it has been radially-compressed for delivery through a catheter 1704 into an aneurysm sac 1706, wherein the flexible mesh has been inserted into the aneurysm sac, and wherein embolic members and/or material 1705 are starting to be delivered through the catheter (and through the annular member) into the flexible mesh and the distal portion of the aneurysm sac. FIG. 20 shows the flexible mesh and the distal portion of aneurysm sac having been filed with embolic members and/or material and the catheter having been removed.

In this example, an annular member is a ring or band which encircles a middle portion (between the ends) of the tubular mesh. In an example, an annular member can be a metal ring, band, or cylinder. In an example, an annular member can be a polymer ring, band, or cylinder. In an example, an annular member can be a wire, cord, or string. In an example, an annular member can be a ring or band which encircles a tubular mesh, thereby radially-constraining and/or pinching the tubular mesh but allowing embolic members and/or embolic material to pass through it into the interior and/or a concavity of the flexible net or mesh. In an example, an annular member can be a cylinder which encircles a tubular mesh, thereby radially-constraining and/or pinching the tubular mesh but allowing embolic members and/or embolic material to pass through it into the interior and/or a concavity of the flexible net or mesh.

In an example, an annular member can be a cord or wire which encircles a tubular mesh, thereby radially-constraining and/or pinching the tubular mesh but allowing embolic members and/or embolic material to pass through it into the interior and/or a concavity of the flexible net or mesh. In an example, an annular member can be a catheter or tube around which a tubular mesh is attached, thereby radially-constraining and/or pinching the tubular mesh but allowing embolic members and/or embolic material to pass through it into the interior and/or a concavity of the flexible net or mesh. In an example, an annular member can be a lumen through a flexible net or mesh through which embolic members and/or material is inserted into the flexible net or mesh.

In an example, a tubular mesh can be soldered, melted, glued, or crimped onto an annular member. In an example, an annular member can have an inner ring and an outer ring, wherein a tubular mesh is fixed (e.g. soldered, melted, glued, or crimped) between the two rings. In an example, an annular member can comprise an inner ring or cylinder and an outer elastic band, wherein the tubular mesh is held between the inner and outer portions. In this example, an annular member can be centrally-located with respect to a proximal surface of the flexible net or mesh. In an example, an annular member can be centrally-located with respect to the longitudinal axis of the flexible net or mesh. In an example, an annular member can be a hub into which proximal ends of braided wires or tubes of the stent are bound or attached. In an example, an annular member can be off-axial with respect to the longitudinal axis of the flexible net or mesh.

In an example, an annular member can comprise two nested and/or concentric (inner and outer) cylinders, wherein the tubular mesh is pinched and/or crimped between the two cylinders. In an example, an annular member can comprise two nested and/or concentric (inner and outer) rings or bands, wherein the tubular mesh is pinched and/or crimped between the two rings or bands. In an example, an annular member can comprise two nested and/or concentric (inner and outer) cylinders, wherein the tubular mesh is melted or glued between the two cylinders. In an example, an annular member can comprise two nested and/or concentric (inner and outer) rings or bands, wherein the tubular mesh is melted or glued between the two rings or bands.

In an example, an annular member can be a catheter which extends through the proximal surface of a flexible net or mesh, wherein the catheter is detached and/or removed after embolic members and/or material has been inserted through the catheter into the interior or distal-facing concavity of the flexible net or mesh. In an example, a distal portion of the catheter used to deliver embolic members and/or material can extend through the proximal surface of a flexible net or mesh and be detached from the rest of the catheter after embolic members and/or material has been inserted through the catheter. In an example, an annular member can be attached to a catheter during delivery of embolic members and/or material, and then detached (e.g. by the application of electromagnetic energy) from the catheter after delivery of the embolic members and/or material.

In an example, an annular member can have an outer diameter which is between 5% and 20% of the diameter of the tubular mesh before the tubular mesh is radially constrained. In an example, an annular member can have an outer diameter which is between 10% and 33% of the diameter of the tubular mesh before the tubular mesh is radially constrained. In an example, an annular member can have an outer ring (or cylinder) with a first diameter and an inner ring (or cylinder) with a second diameter, wherein the tubular mesh is crimped or pinched between the outer ring (or cylinder) and inner ring (or cylinder), and wherein the first diameter is between 50% and 75% of the second diameter. In an example, an annular member can have an outer ring (or cylinder) with a first diameter and an inner ring (or cylinder) with a second diameter, wherein the tubular mesh is crimped or pinched between the outer ring (or cylinder) and inner ring (or cylinder), and wherein the first diameter is between 66% and 90% of the second diameter.

In an example, an annular member can comprise two nested rings, bands, or cylinders, wherein a section of the tubular mesh is inserted and held between the nested rings, bands, or cylinders. In an example, an annular member can comprise an outer ring, band, or cylinder and an inner ring, band, or cylinder, wherein a section of the tubular mesh is inserted and held between them. In an example, an annular member can comprise an outer ring, band, or cylinder and an inner ring, band, or cylinder, wherein one or both of the rings, bands, or cylinders are threaded. In an example, an annular member can comprise an outer ring, band, or cylinder and an inner ring, band, or cylinder, wherein one or both of the rings, bands, or cylinders has a helical thread. In an example, an annular member can comprise an outer ring, band, or cylinder and an inner ring, band, or cylinder, wherein one or both of the rings, bands, or cylinders has a helical thread to hold a section of the tubular mesh.

In an example, this device can further comprise a closure mechanism which closes an opening through an annular member. The closure mechanism can be closed after embolic members and/or material has been inserted into a flexible net or mesh. In an example, this closure mechanism can be selected from the group consisting of: valve; electric detachment mechanism; elastic ring or band; threaded mechanism; sliding cover; sliding plug; filament loop; and electromagnetic solenoid. In an example, a closure mechanism can be a leaflet valve. In an example, a closure mechanism can be a one-way valve. In an example, a valve can allow embolic members and/or material to enter a flexible net or mesh through an opening in an annular member, but not allow the embolic members and/or material to exit the net or mesh.

In an example, a tubular mesh can be made from a polymer. In an example, a tubular mesh can be woven or braided from polymer threads, filaments, yarns, or strips. In an example, a tubular mesh can be 3D printed. In an example, a flexible net or mesh can be made from a flexible polymer. In an example, a flexible net or mesh can be made from an elastic and/or stretchable polymer. In an example, a flexible net or mesh can be elastic and/or stretchable and can expand as it is filled with embolic members and/or material. In an example, a flexible net or mesh can be sufficiently flexible to conform to the shape of even an irregularly-shaped aneurysm sac as the net or mesh is filled with embolic members and/or material. In an example, a flexible net or mesh can be sufficiently flexible to conform to the shape of even an irregularly-shaped (e.g. non-spherical) aneurysm sac as the net or mesh is filled with embolic members and/or material. In an example, a tubular mesh can be made from one or more materials selected from the group consisting of: Dacron, elastin, hydroxy-terminated polycarbonate, methylcellulose, nylon, PDMS, polybutester, polycaprolactone, polyester, polyethylene terephthalate, polypropylene, polytetrafluoroethene, polytetrafluoroethylene, polyurethane, silicone, and silk.

In an example, a tubular mesh can be made from metal. In an example, a tubular mesh can be made from Nitinol. In an example, a tubular mesh can be a flexible metal mesh. In an example, a tubular mesh can be a braided metal mesh. In an example, a tubular mesh can be woven or braided from metal filaments, wires, or tubes. In an example, a tubular mesh can be made from shape-memory material. In an example, a tubular mesh can be made with both metal and polymer components.

In an example, openings or holes in a flexible net or mesh can be smaller than the size (e.g. diameter, width, and/or length) of embolic members and/or material which is inserted into the net or mesh so that the embolic members and/or material do not escape out of the net or mesh. In an example, openings or holes in a flexible net or mesh can less than half of the size (e.g. diameter, width, and/or length) of embolic members and/or material which is inserted into the net or mesh so that the embolic members and/or material do not escape out of the net or mesh. In an example, openings or holes in a flexible net or mesh can have a size which is less than half of the smallest diameter and/or width of embolic members and/or material which is inserted into the net or mesh so that the embolic members and/or material do not escape out of the net or mesh. In an example, openings or holes in a flexible net or mesh can have a size which less than half of the smallest length of embolic members and/or material which is inserted into the net or mesh so that the embolic members and/or material do not escape out of the net or mesh.

In an example, a tubular mesh can have hexagonal openings In an example, a tubular mesh with hexagonal openings can be made using 3D printing. In an example, a flexible metal tubular mesh with hexagonal openings can be made by 3D printing with liquid metal. In an example, a tubular mesh with hexagonal openings can be made by 3D printing with a polymer. In an example, a tubular mesh with hexagonal openings can be made by 3D printing with an elastomeric polymer. In an example, a tubular mesh with hexagonal openings can be made by 3D printing with a silicone-based polymer. In an example, a tubular mesh with hexagonal openings can be made by 3D printing with polydimethylsiloxane (PDMS).

In an example, a tubular mesh can have quadrilateral openings. In an example, a tubular mesh with quadrilateral openings can be made using 3D printing. In an example, a flexible metal tubular mesh with quadrilateral openings can be made by 3D printing with liquid metal. In an example, a tubular mesh with quadrilateral openings can be made by 3D printing with a polymer. In an example, a tubular mesh with quadrilateral openings can be made by 3D printing with an elastomeric polymer. In an example, a tubular mesh with quadrilateral openings can be made by 3D printing with a silicone-based polymer. In an example, a tubular mesh with quadrilateral openings can be made by 3D printing with polydimethylsiloxane (PDMS).

In an example, a tubular mesh can have circular openings. In an example, a tubular mesh with circular openings can be made using 3D printing. In an example, a flexible metal tubular mesh with circular openings can be made by 3D printing with liquid metal. In an example, a tubular mesh with circular openings can be made by 3D printing with a polymer. In an example, a tubular mesh with circular openings can be made by 3D printing with an elastomeric polymer. In an example, a tubular mesh with circular openings can be made by 3D printing with a silicone-based polymer. In an example, a tubular mesh with circular openings can be made by 3D printing with polydimethylsiloxane (PDMS).

In an example, a tubular mesh can be made with a cobalt chromium alloy. In an example, a tubular mesh can be made with a nickel-titanium alloy. In an example, a tubular mesh can comprise cobalt chromium alloy wires, filaments, or tubes. In an example, a tubular mesh can comprise nickel-titanium alloy wires, filaments, or tubes. In an example, a tubular mesh can comprise nitinol wires, filaments, or tubes. In an example, a tubular mesh can be made with nitinol. In an example, a tubular mesh can comprise platinum wires, filaments, or tubes. In an example, a tubular mesh can be made with platinum. In an example, a tubular mesh can comprise stainless steel wires, filaments, or tubes. In an example, a tubular mesh can be made with stainless steel. In an example, a tubular mesh can comprise tantalum wires, filaments, or tubes. In an example, a tubular mesh can be made with tantalum.

In an example, a mid-section of tubular mesh can be more flexible than the proximal and distal sections of the tubular mesh to predispose the tubular mesh to fold over itself at that mid-section to more easily form a dual-layer flexible net or mesh via inversion or eversion of the tubular mesh. In an example, a mid-section of tubular mesh can be more flexible than the proximal and distal sections of the tubular mesh to predispose the tubular mesh to fold over itself at that mid-section to more easily form a dual-layer bowl-shaped flexible net or mesh via inversion or eversion of the tubular mesh. In an example, a mid-section of tubular mesh can be less dense than the proximal and distal sections of the tubular mesh to predispose the tubular mesh to fold over itself at that mid-section to more easily form a dual-layer flexible net or mesh via inversion or eversion of the tubular mesh.

In an example, a mid-section of tubular mesh can be less dense than the proximal and distal sections of the tubular mesh to predispose the tubular mesh to fold over itself at that mid-section to more easily form a dual-layer bowl-shaped flexible net or mesh via inversion or eversion of the tubular mesh. In an example, a mid-section of tubular mesh can be made with a lower-durometer material than the proximal and distal sections of the tubular mesh to predispose the tubular mesh to fold over itself at that mid-section to more easily form a dual-layer flexible net or mesh via inversion or eversion of the tubular mesh. In an example, a mid-section of tubular mesh can be made with a lower-durometer material than the proximal and distal sections of the tubular mesh to predispose the tubular mesh to fold over itself at that mid-section to more easily form a dual-layer bowl-shaped flexible net or mesh via inversion or eversion of the tubular mesh.

In an example, a distal portion (e.g. the distal half) of a flexible net or mesh can have a lower durometer than the proximal portion (e.g. the proximal half) of the flexible net or mesh. In an example, a distal portion (e.g. the distal half) of a flexible net or mesh can be more flexible than the proximal portion (e.g. the proximal half) of the flexible net or mesh. In an example, a distal portion (e.g. the distal half) of a flexible net or mesh can be less dense than the proximal portion (e.g. the proximal half) of the flexible net or mesh. In an example, a distal portion (e.g. the distal half) of a flexible net or mesh can be more porous than the proximal portion (e.g. the proximal half) of the flexible net or mesh.

In an example, a flexible net or mesh can be folded and/or compressed as it is delivered through a catheter to an aneurysm sac. In an example, a flexible net or mesh can have radial folds as it is delivered through a catheter to an aneurysm sac. In an example, a flexible net or mesh can have longitudinal folds as it is delivered through a catheter to an aneurysm sac. In an example, a flexible net or mesh can have cross-sectional folds as it is delivered through a catheter to an aneurysm sac.

In an example, a flexible net or mesh can have a longitudinal axis which spans in a proximal-to-distal direction. Proximal can be defined as being closer to the point of entry into a person's body during delivery through the person's vasculature (in the catheter) to the aneurysm and closer to the aneurysm neck after insertion into the aneurysm sac. In this example, a tubular mesh is transformed into a “ball in a bowl” flexible net or mesh by: radially constraining a mid-section (or proximal one-third section) of the tubular mesh with a mid-section annular member; radially constraining the distal end of the tubular mesh with a distal annular member; and everting the proximal portion of the tubular mesh over the upper globe shape between the mid-section and the distal end.

Alternatively, a tubular mesh can be transformed into a double-layer, distally-concave, bowl-shaped flexible net or mesh by a single annular member in a middle section (between the ends) of the tubular mesh which radially-constrains the middle of the tubular mesh, wherein the proximal portion of the mesh is everted distally over the distal portion of the mesh until it has a distally-concave shape. In an example, the distal circumference of the flexible net or mesh comprises two nested tubular openings. In an example, a tubular mesh can be transformed into a single-layer, distally-concave, bowl-shaped flexible net or mesh by a single annular member which radially-constrains the proximal end of the tubular mesh. In an example, a tubular mesh can be transformed into single-layer, proximally-concave, bowl-shaped flexible net or mesh by a single annular member which radially-constrains the distal end of the tubular mesh.

In an example, a tubular mesh can be transformed into a double-layer, distally-concave, bowl-shaped flexible net or mesh by two annular members (a proximal annular member and a distal annular member) which radially-constrain the proximal and distal ends of the tubular mesh, wherein the distal portion of the tubular mesh is inverted proximally (e.g. folded proximally) until it has a distally-concave shape. In an example, the distal circumference of the flexible net or mesh can be a fold in the net or mesh. In an example, proximal and distal annular members can be aligned so that embolic members and/or material can be delivered through them into the distal-facing concavity of the double-layer bowl-shaped flexible net or mesh. In an example, both of the radially-constrained ends can project into the interior of flexible net or mesh. In an example, the proximal end can be inverted to project into the interior of bowl-shaped flexible net or mesh and the distal end is not.

In an example, a tubular mesh can be transformed into a single-layer ellipsoidal and/or generally globular flexible net or mesh by two annular members which radially-constrain the proximal and distal ends of the tubular mesh. In an example, both of these radially-constrained ends can be inverted to project into the interior of flexible net or mesh. In an example, the proximal end can be inverted to project into the interior of flexible net or mesh and the distal end can remain outside the interior of the flexible net or mesh. In an example, a tubular mesh is transformed into single-layer spherical flexible net or mesh by two annular members which radially-constrain the proximal and distal ends of the tubular mesh.

In an example, bound and/or inverted ends of a flexible net or mesh can both extend into the interior of a flexible net or mesh in a spherical, ellipsoidal, and/or globular configuration. In an example, a distal bound and/or inverted end of a flexible net or mesh can extend into the interior of a flexible net or mesh in a spherical, ellipsoidal, and/or globular configuration and a proximal bound and/or inverted end of the flexible net or mesh can extend outward from a flexible net or mesh in a spherical, ellipsoidal, and/or globular configuration. In an example, a proximal bound and/or inverted end of a flexible net or mesh can extend into the interior of a flexible net or mesh in a spherical, ellipsoidal, and/or globular configuration and a distal bound and/or inverted end of the flexible net or mesh can extend outward from a flexible net or mesh in a spherical, ellipsoidal, and/or globular configuration.

In an example, a tubular mesh can be made from polycarbonate urethane (PCU). In an example, a tubular mesh can be made from polydimethylsiloxane (PDMS). In an example, a tubular mesh can be made from polyesters. In an example, a tubular mesh can be made from polyether block amide (PEBA). In an example, a tubular mesh can be made from polyetherether ketone (PEEK). In an example, a tubular mesh can be made from polyethylene. In an example, a tubular mesh can be made from polyethylene glycol (PEG). In an example, a tubular mesh can be made from polyethylene terephthalate (PET).

In an example, a tubular mesh can be made from polyglycolic acid (PGA). In an example, a tubular mesh can be made from polylactic acid (PLA). In an example, a tubular mesh can be made from poly-N-acetylglucosamine (PNAG). In an example, a tubular mesh can be made from polyolefin. In an example, a tubular mesh can be made from polyoleandlena. In an example, a tubular mesh can be made from polypropylene. In an example, a tubular mesh can be made from polytetrafluoroethylene (PTFE). In an example, a tubular mesh can be made from polyurethane (PU). In an example, a tubular mesh can be made from polywanacrakor. In an example, a tubular mesh can be made from polyvinyl alcohol (PVA). In an example, a tubular mesh can be made from polyvinyl pyrrolidone (PVP).

In an example, a tubular mesh from which a flexible net or mesh is formed can be tapered. In an example, the distal end of a tubular mesh can have a smaller diameter than the proximal end of the tubular mesh. In an example, the distal end of a tubular mesh can have a larger diameter than the proximal end of the tubular mesh. In an example, a tubular mesh from which a flexible net or mesh is formed can have differential flexibility. In an example the distal portion of a tubular mesh can have a first level of flexibility and the proximal portion of the tubular mesh can have a second level of flexibility, wherein the first level is less than the second level. In an example the distal portion of a tubular mesh can have a first level of flexibility and the proximal portion of the tubular mesh can have a second level of flexibility, wherein the first level is greater than the second level.

In an example, a tubular mesh from which a flexible net or mesh is formed can have differential porosity. In an example the distal portion of a tubular mesh can have a first porosity level and the proximal portion of the tubular mesh can have a second porosity level, wherein the first level is less than the second level. In an example the distal portion of a tubular mesh can have a first porosity level and the proximal portion of the tubular mesh can have a second porosity level, wherein the first level is greater than the second level. In an example, a tubular mesh from which a flexible net or mesh is formed can have differential durometer. In an example the distal portion of a tubular mesh can have a first durometer level and the proximal portion of the tubular mesh can have a second durometer level, wherein the first level is less than the second level. In an example the distal portion of a tubular mesh can have a first durometer level and the proximal portion of the tubular mesh can have a second durometer level, wherein the first level is greater than the second level.

In an example, the width of a flexible net or mesh in a bowl-shaped configuration can be larger than the width of the aneurysm neck. In an example, the circumference of a flexible net or mesh in a bowl-shaped configuration can be larger than the circumference of the aneurysm neck. In an example, the width of a flexible net or mesh in a bowl-shaped configuration can be at least 10% larger than the width of the aneurysm neck. In an example, the circumference of a flexible net or mesh in a bowl-shaped configuration can be at least 10% larger than the circumference of the aneurysm neck. In an example, the width of a flexible net or mesh in a bowl-shaped configuration can be at least 90% of the maximum width of the aneurysm sac (parallel to the aneurysm neck). In an example, the circumference of a flexible net or mesh in a bowl-shaped configuration can be at least 90% of the circumference of the maximum circumference of the aneurysm sac (parallel to the aneurysm neck). In an example, a flexible net or mesh can function as a neck bridge, reducing or completely blocking blood flow from the parent vessel into the aneurysm sac.

In an example, a flexible net or mesh formed from a tubular mesh can have a generally-hemispherical shape after a tubular mesh has been radially-constrained by one or more annular members. In an example, a flexible net or mesh formed from a tubular mesh can have a generally globular and/or spherical shape after a tubular mesh has been radially-constrained by one or more annular members. In an example, a flexible net or mesh formed from a tubular mesh can have an ellipsoidal or oval shape after a tubular mesh has been radially-constrained by one or more annular members. In an example, a flexible net or mesh formed from a tubular mesh can have a disk shape after a tubular mesh has been radially-constrained by one or more annular members.

In an example, a flexible net or mesh formed from a tubular mesh can have the shape of a paraboloid-of-revolution (e.g. a paraboloid revolved around a left or right vertical edge) after a tubular mesh has been radially-constrained by one or more annular members. In an example, a flexible net or mesh formed from a tubular mesh can comprise a carlavian curve shape after a tubular mesh has been radially-constrained by one or more annular members. In an example, a flexible net or mesh formed from a tubular mesh can have a toroidal shape after a tubular mesh has been radially-constrained by one or more annular members. In an example, a flexible net or mesh formed from a tubular mesh can have a half-toroidal shape (e.g. a sliced bagel shape) after a tubular mesh has been radially-constrained by one or more annular members.

In an example, the distal end of a tubular mesh can be radially-constrained by a distal annular member and the proximal end of a tubular mesh can be radially-constrained by a proximal annular member to form a generally-globular, spherical, and/or ellipsoidal flexible net or mesh which is inserted into an aneurysm sac. In an example, the distal end of a tubular mesh can be radially-constrained by a distal annular member and the proximal end of a tubular mesh can be radially-constrained by a proximal annular member to form a generally-globular, spherical, and/or ellipsoidal shape, wherein the distal portion is then inverted and/or folded to create a two-layer bowl-shaped flexible net or mesh which is inserted into an aneurysm sac. In an example, both the distal end of a tubular mesh and the proximal end of a tubular mesh can be radially-constrained by a proximal annular member to form a two-layer bowl-shaped flexible net or mesh which is inserted into an aneurysm sac.

In an example a flexible net or mesh can be a two-layer bowl-shaped mesh with a distally-concave proximal layer and a distally-concave distal layer. In an example a flexible net or mesh can be a two-layer bowl-shaped mesh with a distally-concave proximal layer and a distally-concave distal layer, wherein the distance between the proximal and distal layers is greater in a radially-central portion of the flexible net or mesh than in radially-peripheral portions of the flexible net or mesh. In an example a flexible net or mesh can be a two-layer bowl-shaped mesh with a proximal layer and a distal layer, wherein the proximal layer has a uniform distal-facing concavity, but the distal layer has locally-concave and locally-convex portions. In an example, the radially-central portion of the distal layer is locally-convex and the radially-peripheral portions of the distal layer are locally-concave. In an example, the radially-central portion of the distal layer is less distally-concave than the radially-peripheral portions of the distal layer.

In an example, embolic members and/or material which is inserted into the flexible net or mesh can be microspheres or microballs. In an example, embolic members and/or material inserted into the flexible net or mesh can be microsponges. In an example, embolic members and/or material inserted into the flexible net or mesh can be pieces of foam. In an example, embolic members and/or material inserted into the flexible net or mesh can be microbeads. In an example, embolic members and/or material inserted into the flexible net or mesh can be pieces of hydrogel. In an example, embolic members and/or material inserted into the flexible net or mesh can be metal embolic coils. In an example, embolic members and/or material inserted into the flexible net or mesh can be embolic ribbons. In an example, embolic members and/or material inserted into the flexible net or mesh can be yarns or filaments. In an example, embolic members and/or material can be polymer strands or coils. In an example, accumulation of embolic members and/or material in an aneurysm sac can compress a flexible net or mesh from a spherical, ellipsoidal, and/or globular configuration into a hemispherical, bowl-shaped, and/or distally-concave configuration by pressing against the distal surface of the flexible net or mesh.

In an example, embolic members and/or material inserted into the flexible net or mesh can be microspheres or microballs connected by a longitudinal wire, cord, and/or filament (e.g. in a “string-of-pearls” configuration). In an example, embolic members and/or material inserted into the flexible net or mesh can be microsponges connected by a longitudinal wire, cord, and/or filament (e.g. in a “string-of-pearls” configuration). In an example, embolic members and/or material inserted into the flexible net or mesh can be pieces of foam connected by a longitudinal wire, cord, and/or filament (e.g. in a “string-of-pearls” configuration). In an example, embolic members and/or material inserted into the flexible net or mesh can be microbeads connected by a longitudinal wire, cord, and/or filament (e.g. in a “string-of-pearls” configuration).

In an example, embolic members and/or material inserted into the flexible net or mesh can be pieces of hydrogel connected by a longitudinal wire, cord, and/or filament (e.g. in a “string-of-pearls” configuration). In an example, embolic members and/or material inserted into the flexible net or mesh can be embolic coils connected by a longitudinal wire, cord, and/or filament (e.g. in a “string-of-pearls” configuration). In an example, embolic members and/or material inserted into the flexible net or mesh can be embolic ribbons connected by a longitudinal wire, cord, and/or filament (e.g. in a “string-of-pearls” configuration). In an example, embolic members and/or material inserted into the flexible net or mesh can be yarns or filaments connected by a longitudinal wire, cord, and/or filament (e.g. in a “string-of-pearls” configuration).

In an example, embolic members and/or material inserted into the flexible net or mesh can be liquid which congeals and/or solidifies. In an example, embolic members and/or material inserted into the flexible net or mesh can be a polymer which congeals and/or solidifies. In an example, embolic members and/or material inserted into the flexible net or mesh can be a liquid embolic material. In an example, embolic members and/or material inserted into the flexible net or mesh can be hydrogel material. In an example, embolic members and/or material inserted into the flexible net or mesh can be congealing adhesive material. In an example, accumulation of embolic members and/or material in an aneurysm sac can compress a flexible net or mesh from a spherical, ellipsoidal, and/or globular configuration to a hemispherical, bowl-shaped, and/or distally-concave configuration by pressing against the distal surface of the flexible net or mesh.

In an example, embolic members and/or material which is inserted through an annular member into a flexible net or mesh can be one or more mesh ribbons. In an example, embolic members and/or material which is inserted through an annular member into a flexible net or mesh can be one or more wire mesh ribbons. In an example, embolic members and/or material which is inserted through an annular member into a flexible net or mesh can be one or more polymer mesh ribbons. In an example, embolic members and/or material which is inserted through an annular member into a flexible net or mesh can be one or more undulating and/or sinusoidal ribbons. In an example, embolic members and/or material which is inserted through an annular member into a flexible net or mesh can be one or more double-layer mesh ribbons.

In an example, embolic members and/or material can be made with a cobalt chromium alloy. In an example, embolic members and/or material can be made with a nickel-titanium alloy. In an example, embolic members and/or material can be cobalt chromium alloy coils or ribbons. In an example, embolic members and/or material can be nickel-titanium alloy coils or ribbons. In an example, embolic members and/or material can be nitinol coils or ribbons. In an example, embolic members and/or material can be made with nitinol. In an example, embolic members and/or material can be platinum coils or ribbons. In an example, embolic members and/or material can be made with platinum. In an example, embolic members and/or material can be stainless steel coils or ribbons. In an example, embolic members and/or material can be made with stainless steel. In an example, embolic members and/or material can be tantalum coils or ribbons. In an example, embolic members and/or material can be made with tantalum.

In an example, embolic members and/or material can be pushed through a catheter into a flexible net or mesh by a pusher wire and/or plug. In an example, liquid embolic material (which congeals after insertion into the net or mesh) can be pushed through a catheter into a flexible net or mesh by fluid pressure. In an example, embolic members can be pushed into a flexible net or mesh by a flow of liquid (e.g. saline solution), wherein the embolic members are retained in the flexible net or mesh and the saline solution escapes out of openings in the flexible net or mesh. In an example, embolic members and/or material can be pushed through a catheter into a flexible net or mesh by a conveyer belt mechanism. In an example, embolic members and/or material can be pushed through a catheter into a flexible net or mesh by a rotating helical delivery mechanism.

In an example, embolic members which are inserted into a net or mesh can be embolic coils or ribbons. In an example, embolic members which are inserted into a net or mesh can be pieces of foam or gel (such as hydrogel). In an example, embolic members which are inserted into a net or mesh can be microballs or microspheres. In an example, embolic members which are inserted into a net or mesh can be microsponges. In an example, embolic members which are inserted into a net or mesh can be filaments or yarns. In an example, liquid embolic material can be inserted into a net or mesh.

In an example, embolic members which are inserted into a net or mesh can be selected from the group consisting of: pieces of gel; pieces of foam; and micro-sponges. In an example, embolic members which are inserted into a net or mesh can be pieces of gel, such as hydrogel. In an example, embolic members which are inserted into a net or mesh can be pieces of foam. In an example, embolic members which are inserted into a net or mesh can be micro-sponges. In an example, embolic members which are inserted into a net or mesh can be microscale gel balls. In an example, embolic members which are inserted into a net or mesh can be microscale foam balls. In an example, embolic members which are inserted into a net or mesh can be microscale sponge balls. In an example, embolic members which are inserted into a net or mesh can be microscale gel polyhedrons. In an example, embolic members which are inserted into a net or mesh can be microscale foam polyhedrons. In an example, embolic members which are inserted into a net or mesh can be microscale sponge polyhedrons.

In an example, embolic members which are inserted into a net or mesh can have generally spherical or globular shapes. In an example, embolic members which are inserted into a net or mesh can have generally prolate spherical, ellipsoidal, or ovaloid shapes. In an example, embolic members which are inserted into a net or mesh can have apple, barrel, or pair shapes. In an example, embolic members which are inserted into a net or mesh can have torus or ring shapes. In an example, embolic members which are inserted into a net or mesh can have disk or pancake shapes. In an example, embolic members which are inserted into a net or mesh can have peanut or hour-glass shapes. In an example, embolic members which are inserted into a net or mesh can be polyhedrons comprised of hexagonal surfaces. In an example, embolic members which are inserted into a net or mesh can be polyhedrons comprised of quadrilateral surfaces. In an example, embolic members which are inserted into a net or mesh can be polyhedrons comprised of triangular surfaces.

In an example, an embolic member can have a shape which is selected from the group consisting of: apple-shaped, barrel-shaped, bulbous, convex, ellipsoidal, globular, oblate spheroid, ovaloid, prolate-spheroid-shaped, spherical, and truncated-sphere-shaped. In an example, an embolic member can have a shape which is selected from the group consisting of: bowl-shaped, concave, hemispherical, and paraboloid of revolution. In an example, an embolic member can have a shape which is selected from the group consisting of: cubic, hexagon-shaped, hexahedron, octagon-shaped, octahedron, pentagonal-shaped, polyhedron-shaped, pyramidal, rectangular, square, and tetrahedronal.

In an example, embolic members which are inserted into a net or mesh can have a (diameter) size within the range of 0.5 to 2 millimeters. In an example, embolic members which are inserted into a net or mesh can have a (diameter) size within the range of 1 to 5 millimeters. In an example, embolic members which are inserted into a net or mesh can have a (diameter) size within the range of 2 to 10 millimeters. In an example, embolic members which are inserted into a net or mesh can have a (diameter) size within the range of 5 to 20 millimeters. In an example, embolic members which are inserted into a net or mesh can have a (diameter) size within the range of 0.5 to 2 microns. In an example, embolic members which are inserted into a net or mesh can have a (diameter) size within the range of 1 to 5 microns. In an example, embolic members which are inserted into a net or mesh can have a (diameter) size within the range of 2 to 10 microns. In an example, embolic members which are inserted into a net or mesh can have a (diameter) size within the range of 5 to 20 microns.

In an example, between 5 and 20 embolic members can be inserted into a net or mesh. In an example, between 10 and 50 embolic members can be inserted into a net or mesh. In an example, between 20 and 100 embolic members can be inserted into a net or mesh. In an example, between 50 and 500 embolic members can be inserted into a net or mesh.

In an example, embolic members which are inserted into a net or mesh can expand in size within the net or mesh. In an example, embolic members can have a first (average) size while being delivered to an aneurysm sac via a micro-catheter and a second (average) size after expansion within the aneurysm sac, wherein the second (average) size is 10% to 50% larger than the first (average) size. In an example, embolic members can have a first (average) size while being delivered to an aneurysm sac via a micro-catheter and a second (average) size after expansion within the aneurysm sac, wherein the second (average) size is 40% to 100% larger than the first (average) size. In an example, embolic members can have a first (average) size while being delivered to an aneurysm sac via a micro-catheter and a second (average) size after expansion within the aneurysm sac, wherein the second (average) size is more than twice the first (average) size.

In an example, embolic members can self-expand within a net or mesh after they are released from a delivery catheter. In an example, embolic members can swell upon hydration from interaction with blood or other body fluid. In an example, embolic members can be expanded within the net or mesh by one or more mechanisms selected from the group consisting of: expansion due to interaction with body fluid; expansion due to application of thermal energy; expansion due to exposure to a chemical agent; and expansion due to exposure to light energy. In an example, embolics can expand by a factor of 2-5 times. In an example, embolics can expand by a factor of 4-10 times. In an example, embolics can expand by a factor of more than 10 times. In an example, embolic members can expand to a sufficiently-large size that they cannot escape from the net or mesh after insertion into the net or mesh.

In an example, three-dimensional embolic members which are inserted into a net or mesh can be soft and compressible. In an example, three-dimensional embolic members which are inserted into a net or mesh can have a durometer less than 50. In an example, three-dimensional embolic members which are inserted into a net or mesh can have an average durometer within the range of 10 to 30. In an example, three-dimensional embolic members which are inserted into a net or mesh can have an average durometer within the range of 25 to 50. In an example, three-dimensional embolic members which are inserted into a net or mesh can have an average durometer which is less than 70.

In an example, embolic members which are inserted into a net or mesh can be made from a polymer. In an example, embolic members which are inserted into a net or mesh can be made from an elastomeric polymer. In an example, embolic members which are inserted into a net or mesh can be made from a silicone-based polymer. In an example, embolic members which are inserted into a net or mesh can be made from polydimethylsiloxane (PDMS).

In an example, an embolic member can further comprise one or more layers made with different materials. In an example, an inner layer of an embolic member can be made from a first material and an outer layer of an embolic member can be made from a second material. In an example, an inner layer of an embolic member can be made from a first material with a first durometer and an outer layer of an embolic member can be made from a second material with a second durometer, wherein the second durometer is less than the first durometer. In an example, an embolic member can have an outer layer which is adhesive. In an example, an embolic member can have an outer layer with an adhesive property which is activated by application of electromagnetic and/or thermal energy. In an example, an embolic member can have an outer layer with an adhesive property which is activated by interaction with blood.

In an example, there can be a first average durometer of embolic members which are inserted into the net or mesh at a first time and a second average durometer of embolic members which are inserted into the net or mesh at a second time, wherein the second average durometer is greater than the first average durometer. In an example, there can be a first average durometer of embolic members which are inserted into the net or mesh at a first time and a second average durometer of embolic members which are inserted into the net or mesh at a second time, wherein the second average durometer is less than the first average durometer.

In an example, there can be a first average length of longitudinal strands between proximal pairs of embolic members which are inserted into a net or mesh at a first time, a second average length of longitudinal strands between proximal pairs of embolic members which are inserted into the net or mesh at a second time, and the second average length can be greater than the first average length. In an example, there can be a first average length of longitudinal strands between proximal pairs of embolic members which are inserted into a net or mesh at a first time, a second average length of longitudinal strands between proximal pairs of embolic members which are inserted into the net or mesh at a second time, and the second average length can be less than the first average length.

In an example, there can be a first set of embolic members which are inserted into a net or mesh at a first time and a second set of embolic members which are inserted into the net or mesh at a second time, wherein the second set of embolic members are closer together than the first set of embolic members. In an example, there can be a first set of embolic members which are inserted into a net or mesh at a first time and a second set of embolic members which are inserted into the net or mesh at a second time, wherein the first set of embolic members are closer together than the second set of embolic members. In an example, there can be a longitudinal series of embolic members connected by one or more longitudinal strands which is inserted into a net or mesh within an aneurysm sac, wherein embolic members in the longitudinal series are progressively closer to each other moving along the length of the series in a distal to proximal manner. In an example, there can be a longitudinal series of embolic members connected by one or more longitudinal strands which is inserted into a net or mesh within an aneurysm sac, wherein embolic members in the longitudinal series are progressively farther from each other moving along the length of the series in a distal to proximal manner.

In an example, embolic members which are inserted into the net or mesh at a first time can have first shapes, embolic members which are inserted into the net or mesh at a second time can have second shapes, and the second shape can be different than the first shape. In an example, embolic members which are inserted into the net or mesh at a first time can be made with a first (combination of) material, embolic members which are inserted into the net or mesh at a second time can be made with a second (combination of) material, and the second (combination of) material can be different from the first (combination of) material. In an example, embolic members which are inserted into the net or mesh at a first time can be made with a first (combination of) material, embolic members which are inserted into the net or mesh at a second time can be made with a second (combination of) material, and the second (combination of) material can be more flexible, elastic, and/or compliant than the first (combination of) material.

In an example, embolic members which are inserted into the net or mesh at a first time can be made with a first (combination of) material, embolic members which are inserted into the net or mesh at a second time can be made with a second (combination of) material, and the second (combination of) material can have a lower durometer than the first (combination of) material. In an example, embolic members which are inserted into the net or mesh at a first time can be made with a first (combination of) material, embolic members which are inserted into the net or mesh at a second time can be made with a second (combination of) material, and the second (combination of) material can be less flexible, elastic, and/or compliant than the first (combination of) material. In an example, embolic members which are inserted into the net or mesh at a first time can be made with a first (combination of) material, embolic members which are inserted into the net or mesh at a second time can be made with a second (combination of) material, and the second (combination of) material can have a higher durometer than the first (combination of) material.

In an example, there can be a first average size of embolic members which are inserted into the net or mesh at a first time, a second average size of embolic members which are inserted into the net or mesh at a second time, and the second average size can be greater than the first average size. In an example, there can be a first average size of embolic members which are inserted into the net or mesh at a first time, a second average size of embolic members which are inserted into the net or mesh at a second time, and the second average size can be less than the first average size.

In an example, a net or mesh can be delivered into an aneurysm sac via a catheter and/or delivery tube. In an example, a plurality of embolic members can be delivered into the net or mesh via the same catheter and/or delivery tube. In an example, a net or mesh can be delivered into an aneurysm sac via a first catheter and/or delivery tube and a plurality of embolic members can be delivered into the net or mesh via a second catheter and/or delivery tube.

In an example, embolic members can be made from ethylene vinyl alcohol (EVA). In an example, embolic members can be made from polyolefin. In an example, embolic members can be made from fibrinogen. In an example, embolic members can be made from polylactic acid (PLA). In an example, embolic members can be made from polyethylene terephthalate (PET). In an example, embolic members can be made from steel (e.g. stainless steel). In an example, embolic members can be made from methylcellulose.

In an example, embolic members can be made from acrylic. In an example, embolic members can be made from polyethylene glycol (PEG). In an example, embolic members can be made from silk. In an example, embolic members can be made from alginate. In an example, embolic members can be made from gold. In an example, embolic members can be made from polyethylene. In an example, embolic members can be made from polyoleandlena. In an example, embolic members can be made from tantalum. In an example, embolic members can be made from cobalt-chrome alloy (cobalt chromium).

In an example, embolic members can be made from polyetherether ketone (PEEK). In an example, embolic members can be made from polywanacrakor. In an example, embolic members can be made from thermoplastic elastomer. In an example, embolic members can be made from polycarbonate urethane (PCU). In an example, embolic members can be made from water-soluble synthetic polymer. In an example, embolic members can be made from collagen. In an example, embolic members can be made from polyvinyl alcohol (PVA).

In an example, embolic members can be made from titanium. In an example, embolic members can be made from polyether block amide (PEBA). In an example, embolic members can be made from radiopaque material. In an example, embolic members can be made from copolymer. In an example, embolic members can be made from polyvinyl pyrrolidone (PVP). In an example, embolic members can be made from polydimethylsiloxane (PDMS). In an example, embolic members can be made from zirconium-based alloy. In an example, embolic members can be made from polyesters. In an example, embolic members can be made from hydrogel. In an example, embolic members can be made from silicone. In an example, embolic members can be made from nitinol (or other nickel titanium alloy).

In an example, embolic members can be made from polyglycolic acid (PGA). In an example, embolic members can be made from small intestinal submucosa. In an example, embolic members can be made from nylon. In an example, embolic members can be made from polypropylene. In an example, embolic members can be made from platinum. In an example, embolic members can be made from polyurethane (PU). In an example, embolic members can be made from tungsten. In an example, embolic members can be made from fibrin.

In an example, embolic members can be made from poly-N-acetylglucosamine (PNAG). In an example, embolic members can be made from latex. In an example, embolic members can be made from fibronectin. In an example, embolic members can be made from palladium. In an example, embolic members can be made from polytetrafluoroethylene (PTFE). In an example, embolic members can be made from gelatin.

In an example, a selected quantity, series, length, and/or volume of embolic members can be selectively dispensed and/or detached into the net or mesh in situ by a mechanism selected from the group consisting of: breaking a connection between embolic members in a series of embolic members; cutting a connection between embolic members in a series of embolic members (e.g. with a cutting edge or laser); dissolving a connection between embolic members in a series of embolic members (e.g. with thermal energy or a chemical); electrolytic mechanism; hydraulic mechanism; injecting a flow of embolic members suspended in a liquid or gel into a net or mesh; melting a connection between embolic members in a series of embolic members (e.g. with thermal or light energy); progressing embolic members into a net or mesh via a conveyor belt (e.g. chain-based conveyor); progressing embolic members into a net or mesh via a helical conveyor (e.g. with an Archimedes' screw); pushing embolic members into a net or mesh using the force of a liquid flow; pusher rod and/or plunger; release detachment mechanism; and thermal detachment mechanism.

In an example, embolic members can differ among themselves with respect to one or more characteristics selected from the group consisting of: porosity, shape, size, material, composition, coating, radiopacity, strength, stiffness, and type. In an example, a plurality of embolic members can be delivered into a net or mesh in a linear (longitudinal) array or series of inter-connected embolic members. In an example, a plurality of embolic members can be delivered into a net or mesh in a linear (longitudinal) array of connected embolic members, wherein this linear array can be cut, separated, and/or detached in situ (in a remote manner) at one or more selected locations by the user of the device in order to deliver a selected quantity, length, or volume or embolic members. In an example, a plurality of embolic members can be delivered into a net or mesh in a planar array of inter-connected embolic members. In an example, a plurality of embolic members can be delivered into a net or mesh in a three-dimensional array of inter-connected embolic members.

In an example, a series of embolic members can be delivered into a net or mesh, wherein successive embolic members in the series are closer together. In an example, a series of embolic members can be delivered into a net or mesh, wherein embolic members in the series are progressively closer together (as one progresses along the series in a distal to proximal manner). In an example, a series of embolic members can be delivered into a net or mesh, wherein successive embolic members in the series are farther apart from each other. In an example, a series of embolic members can be delivered into a net or mesh, wherein embolic members in the series are progressively farther apart (as one progresses along the series in a distal to proximal manner).

In an example, a series of embolic members can be delivered into a net or mesh, wherein successive embolic members in the series decrease in durometer. In an example, a series of embolic members can be delivered into a net or mesh, wherein embolic members in the series have progressively lower durometer values (as one progresses along the series in a distal to proximal manner). In an example, a series of embolic members can be delivered into a net or mesh, wherein successive embolic members in the series increase in durometer. In an example, a series of embolic members can be delivered into a net or mesh, wherein embolic members in the series have progressively higher durometer values (as one progresses along the series in a distal to proximal manner).

In an example, a series of embolic members can be delivered into a net or mesh, wherein successive embolic members in the series are made of different materials. In an example, a series of embolic members can be delivered into a net or mesh, wherein successive embolic members in the series are made of different materials, wherein these materials differ in porosity. In an example, a series of embolic members can be delivered into a net or mesh, wherein successive embolic members in the series are made of different materials, wherein these materials differ in radiopacity. In an example, a series of embolic members can be delivered into a net or mesh, wherein successive embolic members in the series are made of different materials, wherein these materials differ in stiffness. In an example, a series of embolic members can be delivered into a net or mesh, wherein successive embolic members in the series are made of different materials, wherein these materials differ in durometer.

In an example, a series of embolic members can be delivered into a net or mesh, wherein successive embolic members in the series decrease in porosity. In an example, a series of embolic members can be delivered into a net or mesh, wherein embolic members in the series become progressively less porous (as one progresses along the series in a distal to proximal manner). In an example, a series of embolic members can be delivered into a net or mesh, wherein successive embolic members in the series increase in porosity. In an example, a series of embolic members can be delivered into a net or mesh, wherein embolic members in the series become progressively more porous (as one progresses along the series in a distal to proximal manner).

In an example, a series of embolic members can be delivered into a net or mesh, wherein successive embolic members in the series differ in shape. In an example, a series of embolic members can be delivered into a net or mesh, wherein successive embolic members in the series differ in their degree of convexity. In an example, a series of embolic members can be delivered into a net or mesh, wherein successive embolic members in the series differ in their degree of concavity.

In an example, a series of embolic members can be delivered into a net or mesh, wherein successive embolic members in the series decrease in size. In an example, a series of embolic members can be delivered into a net or mesh, wherein embolic members in the series become progressively smaller (as one progresses along the series in a distal to proximal manner). In an example, a series of embolic members can be delivered into a net or mesh, wherein successive embolic members in the series increase in size. In an example, a series of embolic members can be delivered into a net or mesh, wherein embolic members in the series become progressively larger (as one progresses along the series in a distal to proximal manner).

In an example, embolic members can be soft, compressible members such as microsponges or blobs of gel. In an example, embolic members can be made from sponge, foam, or gel. In an example, embolic members can be hard, uncompressible members such as hard polymer spheres or beads. In an example, embolic members can be made from one or more materials selected from the group consisting of: cellulose, collagen, acetate, alginic acid, carboxy methyl cellulose, chitin, collagen glycosaminoglycan, divinylbenzene, ethylene glycol, ethylene glycol dimethylmathacrylate, ethylene vinyl acetate, hyaluronic acid, hydrocarbon polymer, hydroxyethylmethacrylate, methlymethacrylate, polyacrylic acid, polyamides, polyesters, polyolefins, polysaccharides, polyurethane, polyvinyl alcohol, silicone, urethane, and vinyl stearate.

In an example, embolic members can have a shape selected from the group consisting of: ball or sphere, ovoid, ellipsoid, and polyhedron. In an example, embolic members can have a Shore OO value, indicative of softness or hardness, within a range of 5 to about 50. In an example, embolic members can have a diameter or like size within a range of 50 micrometers to 2000 micrometers. In an example, differently-sized embolic members can be used. In an example two or more different sizes of embolic members can be inserted into a net or mesh to occlude an aneurysm. In an example, embolic members can include small balls and large balls. In an example, it may be advantageous to first fill a net or mesh with larger balls and then continue filling the net or mesh with smaller balls. In another example, it may be advantageous to first fill a net or mesh with smaller balls and then continue filling the net or mesh with larger balls.

In an example, an intrasaccular aneurysm occlusion device can be filled with a “string of pearls” string (or wire) connected sequence of embolic members. In an example, an intrasaccular aneurysm occlusion device can include a series of embolic members which are connected by a strand. In an example, a device can include a string of pearls” series of embolic members which are linked by a strand (e.g. a thin flexible member). In an example, a device can include a string of pearls” series of embolic members which are centrally linked by a strand (e.g. a thin flexible member). In an example, a “string of pearls” string-or-wire connected sequence of embolic members can comprise a plurality of embolic members which are separate from each other, but pair-wise connected to each other by at least one string or wire. In an example, a plurality of members can be unevenly-spaced along the longitudinal axis of a flexible member. In an example, uneven spacing of the embolic members can be selected based on the size and shape of an aneurysm to be occluded. In an example, the distances between embolic members can vary. In an example, the space between embolic members can differ for occlusion of narrow-neck aneurysms vs. wide-neck aneurysms. In an example, distances between embolic members can become progressively shorter in a distal to proximal direction.

In an example, a line which connects embolic members can be a wire, spring, or chain. In an example, a connecting line can be a string, thread, band, fiber, or suture. In an example, embolic members can be centrally connected to each other by a connecting line. In an example, the centroids of embolic members can be connected by a connecting line. In an example, expanding arcuate embolic members can slide (e.g. up or down) along a connecting line. In an example, embolic members can slide along a connecting line, but only in one direction. In an example, a connecting line can have a ratchet structure which allows embolic members to slide closer to each other but not slide further apart. In an example, this device can further comprise a locking mechanism which stops embolic members from sliding along a connecting line. In an example, application of electromagnetic energy to a connecting line can fuse the line with the embolic members and stop them from sliding, effectively locking them in proximity to each other.

In an example, embolic members can be conveyed through a lumen to an aneurysm in a fluid flow, wherein the fluid escapes out from a net or mesh and the embolic members are retained within the net or mesh. In an example, embolic members can be conveyed through a lumen to an aneurysm by means of a moving belt or wire loop. In an example, embolic members can be conveyed through a lumen to an aneurysm by means of an Archimedes screw.

In an example, a flexible net or mesh can self-expand to a first extent after being released from a catheter into an aneurysm sac. In an example, the flexible net or mesh can further expand, to a second extent, due to pressure from the accumulation of embolic members and/or embolic material within its interior and/or distal-facing concavity. Other example variations discussed elsewhere in this disclosure or in priority-linked disclosures can also be applied to this example where relevant.

I claim:

1. An intrasacular aneurysm occlusion device comprising: at least one annular member, wherein an annular member is selected from the group consisting of one or more rings, bands, cylinders, tubes, and catheters; a flexible net or mesh, wherein the flexible net or mesh has a spherical, ellipsoidal, generally-globular, hemispherical, and/or bowl-shaped first configuration when it is formed by encircling, pinching, inverting, and/or everting a tubular mesh at one or more longitudinal locations using the at least one annular member; wherein the flexible net or mesh has a radially-compressed second configuration for delivery through a catheter into an aneurysm sac; and wherein the flexible net or mesh is inserted and expanded within the aneurysm sac; and embolic members and/or embolic material which is inserted into the interior and/or the distal-facing concavity of the flexible net or mesh through one or more of the annular members.

2. The device in claim 1 wherein an annular member comprises nested rings, bands, or cylinders, wherein a section of the tubular mesh is inserted and held between an outer ring, band, or cylinder and an inner ring, band, or cylinder, and wherein embolic members and/or material are inserted into the flexible net or mesh through the inner ring, band, or cylinder.

3. The device in claim 1 wherein an annular member comprises one or more threaded or corrugated rings, bands, or cylinders.

4. The device in claim 1 wherein the device further comprises a closure mechanism which an operator uses to close an opening in an annular member after embolic members and/or material has been inserted through the opening into the flexible net or mesh.

5. The device in claim 1 wherein the device further comprises a one-way valve in an opening in an annular member which allows embolic members and/or material to enter the flexible net or mesh, but not exit the flexible net or mesh.

6. The device in claim 1 wherein the flexible net or mesh has a spherical, ellipsoidal, and/or generally-globular first configuration.

7. The device in claim 6 wherein the flexible net or mesh has a single-layer spherical, ellipsoidal, and/or generally-globular first configuration.

8. The device in claim 6 wherein the flexible net or mesh has a double-layer spherical, ellipsoidal, and/or generally-globular first configuration.

9. The device in claim 6 wherein the flexible net or mesh has a spherical, ellipsoidal, and/or generally-globular first configuration and wherein proximal and distal annular members which radially-constrain the proximal and distal ends of the tubular mesh, respectively, are inside the spherical, ellipsoidal, and/or generally-globular flexible net or mesh.

10. The device in claim 1 wherein the flexible net or mesh has a hemispherical and/or bowl-shaped first configuration.

11. The device in claim 10 wherein the flexible net or mesh has a single-layer hemispherical and/or bowl-shaped first configuration formed by radially-constraining the proximal end of the tubular mesh with a ring, band, and/or cylinder.

12. The device in claim 10 wherein the flexible net or mesh has a double-layer hemispherical and/or bowl-shaped first configuration formed by radially-constraining a mid-section of the tubular mesh and everting the proximal portion of the tubular mesh over the distal portion of the tubular mesh.

13. The device in claim 10 wherein the flexible net or mesh has a double-layer hemispherical and/or bowl-shaped first configuration formed by radially-constraining the proximal end of the tubular mesh by a proximal annular member, radially-constraining the distal end of the tubular mesh by a distal annual member, and inverting the distal portion of the tubular mesh into the concavity of the proximal portion of the tubular mesh.

14. The device in claim 10 wherein the flexible net or mesh has a double-layer hemispherical and/or bowl-shaped first configuration formed by radially-constraining the proximal end and distal ends of the tubular mesh by a proximal member, thereby inverting the distal portion of the tubular mesh into the concavity of the proximal portion of the tubular mesh.

15. The device in claim 1 wherein the embolic members and/or material comprises one or more longitudinal metal coils.

16. The device in claim 1 wherein the embolic members and/or material comprises one or more longitudinal mesh ribbons.

17. The device in claim 1 wherein the embolic members and/or material comprises one or more longitudinal polymer strands.

18. The device in claim 1 wherein the embolic members and/or material comprises one or more string-of-pearls embolic strands, wherein a string-of-pearls embolic strand is a plurality of embolic beads or other embolic masses connected by a longitudinal wire, filament, string, cord, yarn, or thread.

19. The device in claim 1 wherein the embolic members and/or material comprises a plurality of hydrogel pieces or microsponges.

20. The device in claim 1 wherein the embolic members and/or material comprises liquid or gel which congeals after delivery into the flexible net or mesh.
